(19) United States
(12) Patent Application Publication
(10) Pub. No.: US 2012/0034193 A1
(43) Pub. Date: Feb. 9, 2012

(54) TREATMENT OF NEUROTROPHIC FACTOR MEDIATED DISORDERS

(76) Inventors: Daryl Rees, Cambridgeshire (GB); Antonia Orsi, Cambridgeshire (GB); Patrick Howson, Cambridgeshire (GB); Zongqin Xia, Shanghai (CN); Yaer Hu, Shanghai (CN)

(21) Appl. No.: 13/138,251

(22) PCT Filed: Jan. 22, 2010
(86) PCT No.: PCT/GB2010/050098

§ 371 Date: Oct. 31, 2011

Related U.S. Application Data
(60) Provisional application No. 61/147,084, filed on Jan. 24, 2009.

(30) Foreign Application Priority Data
Jan. 24, 2009 (CN) PCT/CN2009/070319

(51) Int. Cl. A61K 35/12; C07J 71/00; A61P 25/00; A61P 25/28; A61P 25/16; A61P 21/00; A61P 27/02; A61P 9/04; A61P 11/06; A61P 25/08; A61P 25/22; A61P 25/24; A61P 25/18; A61P 25/30; A61P 25/32; A61P 25/36; A61P 29/00; A61P 17/06; A61P 1/00; A61P 37/08; A61P 17/04; A61P 1/04; A61P 1/18; A61P 11/02; A61P 11/14; A61P 19/02; A61P 9/10; A61P 37/00; A61P 37/06; A61P 35/00; A61P 19/08; A61P 43/00; A61K 31/58

(52) U.S. Cl. 424/93.7; 514/173; 540/17; 514/171

(57) ABSTRACT

An agent selected from A/B-cis furostane, furostene, spirostane and spirostene steroidal sapogenins and ester, ether, ketone and glycosylated forms thereof is used to induce self-regulated homeostasis of neurotrophic factors (NFs), for example BDNF and/or GDNF, NFs with limited and manageable side effects in a subject, by modulating NFs in a non-toxic manner under homeostatic control. An effective amount of at least one such agent is administered to the subject, particularly in the treatment or prevention of a range of NF-mediated disorders, particularly neurological, psychiatric, inflammatory, allergic, immune and neoplastic disorders, and in the restoration or normalisation of neuronal and other function in or in relation to any damaged or abnormal tissue, including when assisting tissue (for example, skin, bone, eye and muscle) healing and general skin, bone, eye and muscle health.

### FIELD OF THE INVENTION

The present invention relates to the treatment and prevention of neurotrophic factor-mediated disorders, particularly neurological, psychiatric, inflammatory, allergic, immune and neoplastic disorders, and in the restoration or normalisation of neuronal and other function in or in relation to any damaged or abnormal tissue, including when assisting tissue (for example, skin, bone, eye and muscle) healing and general skin, bone, eye and muscle health, to related non-therapeutic methods, and to compounds and compositions for use therein.

### BACKGROUND OF THE INVENTION

Natural neurotrophic factors (NFs) include neurotrophins, TGF-β-super-family, NFs and neurokines, e.g. nerve growth factor (NGF), brain-derived neurotrophic factor (BDNF), ciliary neurotrophic factor (CNTF), neurotrophin 3 (NT-3), neurotrophin 4 (NT-4) and glial-derived neurotrophic factor (GDNF). Neurotrophic factors bind to cell receptors known as neurotrophic factor receptors (NFrs). The NFr TrkA mediates the effects of NGF. The NFr TrkB is activated by BDNF, NT-3 and NT-4. The NFr TrkC is activated only by NT-3. The NFr low affinity NGF receptor (LNGFR or p57) binds all members of the neurotrophin family The NFr for GDNF comprises of two components, the GDNF binding domain (GDNF receptor α1 (GFRα1)) and the receptor tyrosine component Ret. Binding of GDNF to GFRα1 activates Ret.

Abnormal expression of natural NFs is implicated in a range of disorders, and therapies have been devised, based upon putative NF-mimicking or activating activities of small-molecule non-peptide therapeutic agents. In principle, small-molecule non-peptide (including non-polypeptide and non-protein) therapeutic agents generally have a range of advantages over peptide agents, including lower cost and relative ease of manufacturing, easier handling and storage, reduced inherent toxicity, relative ease of delivery to the patient, especially into the brain, and relative ease of optimisation in the research and development stages, in comparison with peptides. Despite substantial interest in peptide NFs, NF-mimics and NF-enhancers as potential drugs, their inherent developmental difficulties, potential toxicity and other problems has been found to severely limit their potential.

A number of small-molecule non-peptides have been proposed for treating certain neurological and psychiatric disorders. The following paragraphs highlight some of the publications. However, these prior proposals are all characterised by substantial adverse side-effects of the agents, which prevents administration of an effective dose, so that in all cases the compound cannot be developed to provide a marketed drug to treat or prevent neurological and psychiatric disorders.

For example, Xaliproden (Sanofi-Aventis) (1-(2-naphthalen-2-ylethyl)-4-[3-(trifluoromethyl)phenyl]-3,6-dihydro-2H-pyridine hydrochloride; MW of salt: 417.5; MW of free base: 381)), a serotonin 5-HT_{1A} receptor agonist, was found later to also activate NGF to some extent. Xaliproden is reported to have completed Phase III clinical trials as a potential treatment for amyotrophic lateral sclerosis (ALS) (Drugs R D. 2003, 4(6), pp. 386-388) and was recently evaluated in a Phase III trial as a potential for Alzheimer's disease. The 5-HT_{1A}, agonist activity, however, produces dose-dependent adverse effects which restrict the use of Xaliproden as a medicine.

4-Methylcatechol (MW 124) has been reported to stimulate neurotrophin synthesis and thus theoretically offers an approach to the treatment of neurodegeneration (Furukawa et al, Advances in Behavioral Biology, 2002, 53, pp. 233-236). However, this agent has been found to produce toxic side effects, probably due to over stimulation of the expression of nerve growth factor (NGF).

Retinoic acid has been reported to increase serum and nerve levels of NGF and to prevent neuropathy in diabetic mice (Arrieta et al., European Journal of Clinical Investigation, 2005, 35, pp. 201-207) and has been suggested to have a possible therapeutic role in neurodegenerative disorders (Mey and McCaffery, The Neuroscientist, 2004, 10, pp. 409-420). However, this agent is known to have serious dose-limiting toxic side-effects.

AMPA receptor potentiators (AMPAkines) are glutamate receptor modulators, and some have been shown to enhance BDNF expression in vivo (Mackowiak et al., Neuropharmacology, 2002, 43, pp. 1-10). Furthermore, two AMPAkines (CX614 and CX546) have been shown to maximally increase BDNF mRNA levels by 6-12 hours post-administration and then decline to near control levels by 48 hours post-administration, despite continued AMPAkine exposure (Lauterborn et al., Journal of Pharmacology and Experimental Therapeutics, 2003, 307, pp. 297-305). Several AMPAkines have been, or are currently, in development for neurological disorders (Price et al., Pharmacology and Therapeutics, 2007, 115, pp. 292-306). However, at least some of these agents have toxic side-effects.

Certain antidepressants, which include those having a primary action as serotonin selective re-uptake inhibitors (SSRIs) and monoamine oxidase inhibitors (MAOIs), have also been shown to increase BDNF mRNA levels in vivo (Malberg and Blendy, Trends in Pharmacological Sciences, 2005, 26, pp. 631-638; Martinez-Turrillas et al., Neuropharmacology, 2005, 49, pp. 1178-1188). See also the review entitled “Neurotrophic effects of antidepressant drugs” by Castren, Current Opinion in Pharmacology, 2004, 4, pp. 58-64. However, all these agents are well known to have many undesirable side-effects.

Immunophillins are a class of immunosuppressants which have been shown to potentiate the activity of neurotrophins (Price et al., Pharmacology and Therapeutics, 2007, 115, pp. 292-306). FK506 (Tacrolimus) has been shown to increase BDNF mRNA levels (Zawadzka and Kaminska, Molecular and Cellular Neuroscience, 2003, 22, pp. 202-209) and BDNF and GDNF protein levels (Tanaka et al., Brain Research, 2003, 970, pp. 250-253) in vivo. However, the whole class has serious dose-limiting toxic side effects.

N⁴-(7-chloro-2-[(E)-2-(2-chloro-phenyl-vinyl)]-quinolin-4-yl)-N,N′-diethyl-pentane-1,4-dione (XIB4035), a GFR*-1 receptor agonist, has been reported as promoting neurite outgrowth in a concentration-dependent manner (Tokugawa et al, Neurochemistry International, 42, 1, Jan. 2003, pp. 81-86). However, this molecule too has dose-limiting side-effects.

These known small-molecule agents thus have NF-mimicking or activating effects to some extent, but have dose-dependent adverse side effects either in pre-clinical models or in the clinic. The side-effects can typically manifest themselves in overt toxicity. This severely restricts the potential utility of the agents in therapies.

There is a general need for development of improved, and in particular non-toxic, small-molecule non-peptide bioactive agents for treatment of neurological and psychiatric disorders.

WO-A-99/16786, WO-A-99/48482, WO-A-99/48507, WO-A-01/23407, WO-A-01/23408, WO-A-02/079221, WO-A-03/082893, WO-A-2005/105108, WO-A-2005/105825 and WO-A-2006/048665, the disclosures of which are incorporated herein by reference, relate to the use of certain small-molecule steroids in the treatment of cognitive dysfunction and certain other neurological and psychiatric disorders. Generally speaking, these active agents are A/B-cis furostane, furostene, spirostane or spirostene steroidal sapogenins and ester, ether, ketone and glycosylated forms thereof, the expression “sapogenins” being understood to include all E and/or F ring opened derivatives, for example pseudosapogenin and dihydrospeudosapogenin forms of the said sapogenins. In the unsaturated (-ene) forms of the compounds, one or more double bond is present at locations which do not affect the A/B-cis motif.

WO-A-03/082893, page 25, lines 5 to 18, reports that at least some of the compounds have been found to slow or reverse certain aspects of neuronal degeneration, including reversing adverse cell body changes and neurite atrophy, reducing the release of NFs such as neurotrophins, TGF-β-super-family NFs and neurokines, and reducing neuronal toxicity and apoptosis. This passage also reports that the neuroprotective and reversal of receptor loss effects are actively regulated effects, in which past deterioration is reversed towards the normal or young state with protection against continued deterioration.

The same document, page 26, lines 8 to 15, further reports that it is believed that one physiological effect of the active agents is the ability to increase the synthesis or release of—or to reduce the rate of degradation of—NFs or their receptors. It is theorised that these effects on growth factors “might be due to an effect of the compound on a cytosolic or nuclear receptor, or the binding of a compound to a promoter region with a consequent effect directly on the rate of production of mRNA for the growth factor, or as a consequence of increasing the production of another material factor.”

The same document, page 20, lines 4 onwards, describes the use of the active agents to treat the psychiatric disorders of autistic syndrome, depression and schizophrenia.

Zhang Y, et al, FEBS Letters, 19 Mar. 2008, 582, Issue 6, pp. 956-960, the disclosure of which is incorporated herein by reference, reports that smilagenin appears to increase GDNF mRNA expression in rat mesencephalic dopaminergic neurones damaged by 1-methyl-4-phenylpyridinium (MPP⁺), as well as the GDNF content in the culture medium, and that smilagenin appears to prevent MPP⁺ induced neuronal damage and atrophy in those neurones. This publication originates from the present inventors and is not prior art in all designated states.

The role of NFs in immune system homeostasis has been the subject of much research in recent years (see, for example, Vega, J A et al, J. Anat. 2003, 203, pp. 1-19, and the references cited therein, the disclosures of all of which are incorporated herein by reference). As explained in more detail in that Vega et al publication, and summarised in Table 2 on page 8, NFs have been shown to have a range of activities in relation to a range of cells involved in the immune system, particularly B-lymphocytes, T-lymphocytes, monocytes/macrophages, neutrophils, eosinophils, basophils, mast cells and haematopoietic cells, as well as platelets and vascular tissue. Homeostatic modulation of NFs provides a valuable technique for treating or preventing immune system disorders.

The role of NFs in inflammation and inflammatory disorders and in allergies has also received much attention. It is known that NGF levels increase during inflammation and allergic responses, as well as in diseases of the immune system (see Stanisz, A M & Stanisz, J A, Ann. N Y Acad. Sci., 2000, 917, pp. 268-272; Otten, U et al, Ann. N.Y. Acad. Sci., 2000, 917, pp. 322-330; also the references cited on page 10, column 2, and page 11, columns 1 and 2 of Vega et al). Homeostatic modulation of NFs provides a valuable technique for treating or preventing inflammation and inflammatory disorders and allergic responses.

As is well known, inflammatory, allergic and immune responses can occur simultaneously and in an inter-related manner, for example in autoimmune diseases and in response to challenge by toxins, parasites and other infective agents. Homeostatic modulation of NFs provides a valuable technique for treating or preventing such conditions.

NGF has been shown to have useful effects in vasculitis-induced rheumatoid arthritis (Tuveri, M. et al, Lancet, 2000 Nov 18, 356, pages 1739-1740; Aloe, L., Arch. Physiol. Biochem., 2001, 109, pages 354-356) and is reported as being considered as a new therapeutic strategy in the blockade of NF overexpression during the allergic or inflammatory process (Vega et al publication cited above, page 12, column 1). For the reasons explained above in relation to neurological disorders, the use of NGF protein is less desirable than the use of small molecules. A small molecule agent for the regulation of NF overexpression would be highly desirable.

WO-A-01/64247, the disclosure of which is incorporated herein by reference, describes a method for the treatment or prevention of neoplastic disorders (cancers) characterised by the expression of NF receptors on the cancer cell surface, particularly trk+ cancer cells. The method involves administering an effective amount of an anti-NF agent (referred to as an anti-neurotrophin or anti-NT agent in the reference), for example anti-NF antibodies, anti-NF antisense polynucleotides or an anti-NF trk mutant. It is stated that a range of cancers including breast, thyroid, colon, lung, ovary, skin, muscle, pancreas, prostate, kidney, reproductive organs, blood, immune system tissues (e.g. spleen, thymus and bone marrow), brain and peripheral nervous system tissues may be treated or prevented in this way. The mode of action is stated to be via highly specific binding of the active agent to the NFs, leading to inhibition of trk receptors by neutralization of the activating NF ligand (page 5, lines 8 to 10).

Innominato, P F et al, J. Pathol., 2001, 194, pages 95-100, the contents of which are incorporated herein by reference, described expression of NFs and NF receptors on the surface of melanoma cells. Skin cancer cells, particularly melanoma cells can therefore be included in the above list of NF-receptor-positive cancer cells.

For the reasons explained above in relation to neurological disorders, the use of antibodies, polynucleotides and anti-NF receptor mutant proteins is less desirable than the use of small molecules (see LeSauteur et al, Nature Biotech., 1996, 14, page 1120). A small molecule agent for the homeostatic regulation of NFs to inhibit the trk receptors of the cancer cells through control of the binding partners for the receptors, analogous to the mode of action of the NF proteins, would be highly desirable.

The present invention is based on our novel finding that the said A/B-cis furostane, furostene, spirostane or spirostene steroidal sapogenin agents, and ester, ether, ketone and glycosylated forms thereof as described below, lead to the modulation of NFs in a non-toxic manner and leaving the normal homeostatic control processes of the subject intact. Thus, the agents induce self-regulated homeostasis of NFs with few side-effects, which if present can be managed and which do not prevent administration of an effective dose. The finding, using an essentially non-toxic, non-peptide, small molecule, of induction of self-regulated homeostatsis—whereby in the unhealthy state one or more NF's are restored (through increased or decreased levels) towards the healthy state without adverse side effects—is unexpected and surprising, and provides significant benefits, as will be discussed in more detail below.

Moreover, we have found that the agents induce self-regulated homeostasis of more than one NF, for example BDNF and GDNF without adverse side-effects. The achievement, by one active agent, of self-regulated homeostasis of more than one NF together without adverse side-effects, is surprising and, to our knowledge, unique in any small-molecule agent. Since it is known that neurones typically require more than one NF for optimal neuroprotection and neurorestoration, this finding in accordance with the present invention provides for substantially improved treatment and prophylaxis of NF-mediated disorders and related conditions.

It is also known that NFs play a role in the healing of tissues including skin, corneal tissue, bone and muscles, and are generally beneficial to skin, bone and muscle health. See, for example, Albers, K. M. et al, Neuroscientist 2007, 13, pages 317-382; Asaumi, K., et al., Bone, 26(6), June 2000, pages 625-633; You, L et al., Investigative Opthalmology & Visual Science, October 2001, 42(11), pages 2496-2504; Cruise, B. A. et al., Developmental Biology, 271, (2004), pages 1-10; Jurjus, A. et al., Burns 33 (2007), 892-907.; Matsuda, H., et al., J. Exp. Med., 187(3), 2 Feb. 1998, pages 297-306; Menetrey, J et al., J. Bone Joint Surg (Br), 82-B(1), January 2000, pages 131-137; Micera, A., et al., Cytokine & Growth Factor reviews, 18, (2007), pages 245-256; Nithya, M., et al., Biochim. Biophys. Acta, 1620, (2003), pages 25-31; Matsuda, H et al, J. Exp. Med. 1998, 187, pages 297-306; Lambiase et al, Invest. Ophthalmol. Vision Sci., 2000, 41, pages 1063-1069. The contents of these publications are incorporated herein by reference.

The findings underlying the present invention are thus also applicable to the healing and wellbeing of tissues including skin, bone, muscles and eye tissue such as corneal tissue. Therefore, the present invention also relates to the restoration or normalisation of neuronal function in, or in relation to, any damaged or abnormal tissue, and the assistance of tissue (for example, skin, bone, eye and muscle) healing and general skin, bone and muscle health, including recovery of muscle and tissues from exercise, exertion or wasting, recovery of skin from the effects of sun exposure, wind exposure, rain exposure, cold exposure, ageing and wrinkling, improving endurance and reducing the feeling of fatigue. Without limitation, the tissue healing that may be assisted by the present invention can include healing of wounds and burns, as described in more detail below.

### BRIEF DESCRIPTION OF THE INVENTION

According to a first aspect of the present invention, there is provided a method of inducing self-regulated homeostasis of neurotrophic factors (NFs) in a subject, by modulating the subject's native NFs in a non-toxic manner under homeostatic control, comprising administering to the subject an effective amount of one or more agent selected from A/B-cis furostane, furostene, spirostane and spirostene steroidal sapogenins and ester, ether, ketone and glycosylated forms thereof. The subject's native NFs may be one or both of BDNF and GDNF.

The method of the first aspect of the invention is such that the induction of self-regulated homeostasis of NFs takes place with limited and manageable side effects.

In one particularly preferred embodiment of the first aspect of the invention, the induced homeostasis modulates two or more of the subject's native NFs, for example BDNF and GDNF, together.

According to a second aspect of the present invention, there is provided an agent selected from A/B-cis furostane, furostene, spirostane and spirostene steroidal sapogenins and ester, ether, ketone and glycosylated forms thereof, for use in a method of inducing self-regulated homeostasis of NFs in a subject by modulating the subject's native NFs in a non-toxic manner under homeostatic control.

The agent for use according to the second aspect of the invention is such that the induction of self-regulated homeostasis of NFs takes place with limited and manageable side effects or adverse side effects.

According to a third aspect of the present invention, there is provided a composition comprising an active agent selected from A/B-cis furostane, furostene, spirostane and spirostene steroidal sapogenins and ester, ether, ketone and glycosylated forms thereof, for use in a method of inducing self-regulated homeostasis of NFs in a subject by modulating the subject's native NFs in a non-toxic manner under homeostatic control.

The composition for use according to the third aspect of the invention is such that the induction of self-regulated homeostasis of NFs takes place with limited and manageable side effects or adverse side effects.

According to a fourth aspect of the present invention, there is provided the use of an agent selected from A/B-cis furostane, furostene, spirostane and spirostene steroidal sapogenins and ester, ether, ketone and glycosylated forms thereof, in the manufacture of a medicament for inducing self-regulated homeostasis of NFs in a subject by modulating the subject's native NFs in a non-toxic manner under homeostatic control.

The use according to the fourth aspect of the invention is such that the induction of self-regulated homeostasis of NFs takes place with limited and manageable side effects or adverse side effects.

The present invention limits adverse side effects, particularly side effects related to overinduction, overstimulation or overenhancement of NFs, for example NGF, side effects related to receptor (ant)agonist action, and side effects related to enzyme binding action.

The present invention may be used in conjunction with methods of treatment of NF-mediated disorders, particularly neurological, psychiatric, inflammatory, allergic, immune and neoplastic disorders, and in the restoration or normalisation of neuronal and other function in or in relation to any damaged or abnormal tissue, including when assisting tissue (for example, skin, bone, eye and muscle) healing and general skin, bone, eye and muscle health, and related non-therapeutic methods, in human and non-human animal subjects.

The term “NF-mediated” used herein is to be understood in a general sense, covering disorders and conditions where neurotrophic factors are understood to play a contributing role to the development, progression or effects of the disorder or condition. Thus, for example, disorders or conditions where the current evidence implicates NF receptors, (ant)agonists thereof or other activators or inhibitors of NFs, such disorders or conditions will be understood as “NF-mediated” according to the present invention. Such disorders and conditions are expected to respond to homeostatic modulation of a human or non-human animal subject's native NFs in accordance with the invention.

The present invention may thus be used in conjunction with the restoration of normal neuronal and other function in any damaged or abnormal tissue, for example in tissue (whether brain tissue or other tissue such as skin, bone, eye and muscle) damaged by injury, by lack of blood, by ageing or (in the case of skin) by wrinkling or by exposure to sun, wind, rain, cold or other damaging media. The restoration of normal neuronal function is typically achieved according to the invention by induction of self-regulated homeostasis of NFs leading to neuroregeneration and improved blood flow, as well as normalisation of neuropathic conditions or neuronal abnormalities such as inflammation in the central nervous system (CNS) or peripheral nervous system (PNS).

The present invention may thus be used in conjunction with the assistance of wound healing, particularly to improve the speed and quality of the healing of skin wounds of humans and other mammals. In this context, “wound” includes all lesions of any origin, for example injuries such as cuts and abrasions, knife wounds, surgical trauma, bruises, burns, ulcers, sores. Both chronic and acute wounds can be treated according to the invention.

The present invention may be used in conjunction with fetal, stem or other cell therapy and tissue transplants, particularly to improve the survival of the transplanted material or the efficacy of the therapy or both. Examples include cell therapy to improve brain function or cellular function in other organs of the body.

Still further, the present invention may be used in non-therapeutic methods for promoting or assisting the wellbeing and general health of tissues such as skin, bone, eye and muscle, promoting recovery of muscle and tissues from exercise, exertion or wasting, promoting recovery of skin from the effects of ageing, wrinkling or exposure to sun, wind, rain, cold or other damaging media. improving endurance and muscular stamina (e.g. in competitive or non-competitive sport) and reducing the feeling of fatigue, by virtue of the benefits of self-regulated homeostasis of NFs in such tissue.

In accordance with the invention, the agents may be administered systemically or locally, as their delivery to the sites of action is found to be generally good. In particular, but without limitation, oral and parenteral (e.g. topical) administration routes are found to be suitable, as discussed in more detail below.

The expression “sapogenin”, used herein, includes all E and/or F ring opened derivatives, for example pseudosapogenin and dihydrospeudosapogenin forms of the said sapogenins, subject of course to such derivatives being possible. In the unsaturated (-ene) forms of the compounds, one or more double bond is present at locations which do not affect the A/B-cis motif. Glycosylated forms of sapogenins are commonly referred to as saponins.

### DETAILED DESCRIPTION OF THE INVENTION

### Introduction

The evidence presented in this application shows that the agents do not bind to a range of receptors and enzymes (see Example 1).

Evidence supporting the effects of the active agents on the induction of NFs or NF-receptors is presented in this application. The evidence (see Examples 2 and 3 below) shows that the activity involves enhanced gene expression of NFs and NF-receptors. As can be seen in Example 2, where the neurones are relatively healthy (basal culture), the enhanced gene expression is transitory and the timescale strongly indicates the involvement of a self-regulatory mechanism.

In the more diseased situation of Example 3, the data show a much more prolonged period of enhanced gene expression, showing that the regulatory mechanism remains intact and the degree of enhancement of the gene expression depends on the needs of the system.

The evidence presented in this application also shows that the active agents provide self-regulated homeostasis of NFs, for example BDNF and GDNF in particular (see Examples 4 to 7 and 18 below). Not only is self-regulated normalisation of one NF, for example BDNF or GDNF, by a non-peptide agent exceptional, but the self-regulated normalisation of two NFs, for example BDNF and GDNF together, by a non-peptide agent is unique. The normalisation of both NFs together appears to lead to a synergistic normalised combination of BDNF and GDNF which is particularly beneficial.

The evidence presented in this application also shows that the active agents increase neuritogenesis in a range of CNS and PNS neurones (see Example 8). Importantly, this neuritogenic effect is not dependent on the presence of exogenous NFs. This shows that the effect of the agents of the present invention is an NF induction, rather than enhancement.

The evidence presented in this application also shows that the active agents activate the same intracellular transduction pathways as NFs (see Example 9). This provides supporting evidence of the NF modulating activity of the agents.

The evidence presented in this application also shows that a range of A/B-cis active agents reduce glutamate-induced damage to cortical neurones and apoptosis of dopaminergic neurones, whereas a sapogenin (diosgenin) of generally similar chemical structure, but not possessing the A/B-cis motif, is inactive (see Examples 10 and 11).

The evidence presented in this application also shows that the agents reverse neuronal damage in a range of neurones, i.e. they are neurorestorative or neuroregenerative (see Example 12).

The evidence presented in this application also shows that the agents are orally administrable (see Examples 13 and 14). The example shows that oral administration of the agents improves recovery of nerve function in a mouse model of motor neurone disease or post-traumatic nerve injury.

The evidence presented in this application also shows that the agents reduce anxiety and restore cognition in aged rats (see Example 15).

The evidence presented in this application also shows that the orally administered agents are delivered to a range of body tissues (see Example 16) and are non-toxic at effective doses (see Example 17).

The evidence presented in this application also shows that the agents reduce parkinsonism in macaques (see Example 18).

The evidence presented in this application shows that the NF or NF-receptor (NFr) mediated activity of the agents does not involve direct binding interactions with a range of receptors and enzymes. For example, the activity is not associated with direct-binding agonism, antagonism or non-(ant)agonistic direct binding at a range of important receptors, including hormone receptors such as oestrogen, progesterone, testosterone and serotonin receptors, nicotinic receptors, muscarinic receptors, adrenergic receptors, narcotic receptors such as cannabinoid and opiate receptors, glutamate receptors such as NMDA, AMPA and kainite receptors and retinoic acid receptors such as Retinoid X receptor. As a result, the physiological effect of the active agents is independent of many of the receptor- and enzyme-mediated side effects found with prior known treatments for neurological and psychiatric disorders. For example, the problems found with many prior treatments of neurological and psychiatric conditions, whereby addictions and dependencies, addictive personality types, prior treatments having receptor or enzyme side effects, and current treatments to break an addiction or dependency, could each contraindicate the treatment of the neurological or psychiatric disorder, are substantially reduced with the agents.

The prior art treatments of psychiatric disorders, although they exert their biochemical modes of action immediately, show beneficial psychiatric effects on a much longer timescale. The effects of the present invention are much more immediate, providing evidence of modulation of NFs in a non-toxic manner under homeostatic control. Thus the present invention is distinguished from the known small-molecule (non-peptide) treatments for psychiatric and neurological disorders.

The dose-response profiles of the agents in the tests of the Examples show a maximum followed by a plateau, which is characteristic of a self-regulatory mechanism (see FIG. 1).

The one or more active agent used in the present invention may be used without exogenous administered neurotrophic factors such as GDNF or BDNF.

New Uses Associated with the Invention

The invention thus enables new uses of the active agents to be identified, for example in terms of (i) disorders to be treated, (ii) classes of individuals to be treated, (iii) combination treatments to use, and (iv) circumstances of safe use.

As far as (i) is concerned, for example, the use of the active agents to treat a range of neurological, psychiatric, inflammatory, allergic, immune and neoplastic disorders and conditions as well as personality and behavioural traits and achieving regeneration or normalisation of neurones, blood flow to neurones, regrowth and healing of damaged tissues (for example, skin, bone, eye or muscle tissue), general health and wellbeing of tissues both in and outside of the brain (for example skin, bone, eye and muscle tissue), recovery of muscle and tissues from exercise, exertion or wasting, improving endurance and reducing the feeling of fatigue, regenerating normal neuronal function and normal neuronal networks, via both pharmaceuticals and functional foods, is now identifiable, as will be discussed in more detail below. This use includes non-therapeutic use to improve neurological or psychological functioning of an individual within the normal range of the population, or general health and wellbeing of and individual, non-therapeutic use to improve skin, bone, eye, muscle and other tissue health, for example promoting recovery of skin from the effects of ageing, wrinkling or exposure to sun, wind, rain, cold or other damaging media, and non-therapeutic use to provide for other aspects of health and wellbeing, including recovery of muscle and tissues from exercise, exertion or wasting, improving endurance and reducing the feeling of fatigue, and the terms “disorders”, “conditions” and “traits” will be understood accordingly.

As far as (ii) is concerned, the finding that the agents of the present invention work via self-regulated homeostasis of NFs, rather than modulation or binding to many receptors or enzymes, allows patients to be treated who are sensitive to adverse side-effects from some enzyme inhibiting drugs or receptor agonist drugs. For example, some dementia (Alzheimer's) patients cannot tolerate cholinesterase inhibitors. Some Parkinson's disease patients cannot tolerate L-dopa, and will suffer side-effects including dyskinesia or neuropsychiatric problems such as risk-taking.

As far as (iii) is concerned, for example, the use of combinations of the agents with other co-agents for treatment of particular disorders, conditions and traits, or particular classes of individuals, is now identifiable, as will be discussed in more detail below. The identification of many of such combinations was previously speculative at best. This use includes non-therapeutic use to improve neurological or psychological functioning of an individual within the normal range of the population, non-therapeutic use to improve skin, bone, eye, muscle and other tissue health, for example promoting recovery of skin from the effects of ageing, wrinkling or exposure to sun, wind, rain, cold or other damaging media, and non-therapeutic use to provide for other aspects of health and wellbeing, including recovery of muscle and tissues from exercise, exertion or wasting, improving endurance and reducing the feeling of fatigue, and the terms “disorders”, “conditions” and “traits” will be understood accordingly.

As far as (iv) is concerned, for example, a new range of circumstances of use outside the clinical and pharmaceutical environment is now identifiable, as will be discussed in more detail below.

New Use (i)—New Treatments

The present invention may be used in a method of (a) treating or preventing neurological, psychiatric, inflammatory, allergic, immune and neoplastic disorders, (b) regenerating and/or normalising neurones and blood flow to neurones, including regenerating neuronal function or neuronal networks, (c) regrowth and healing of damaged tissue, (d) recovery of muscle and tissues from exercise, exertion or wasting, (e) improving endurance and reducing the feeling of fatigue, or (f) treating or preventing abnormal behavioural or personality traits, in a human or non-human mammal in need thereof. The neurological, psychiatric, inflammatory, allergic, immune and neoplastic disorders may be those disclosed in the prior art mentioned above, or may be different from those disorders. For example, the neurological methods may be autistic syndrome, depression and schizophrenia, or may be disorders other than these. Methods for the regeneration or normalisation of neurones and blood flow to neurones, regrowth and healing of damaged tissue, neuronal function or neuronal networks include, for example, post-trauma reconstruction of nerves, tissue grafts and post-surgical reconstruction of nerves (e.g. for reattachment of limbs and fingers), assisting recovery from stroke, transient ischemic attacks (TIAs) or other ischemia, for example assisting recovery of nerve function and blood flow to ischemic tissue, assisting the healing of wounds, bone and muscle, and treating neuropathy and any inflammatory condition relating to the CNS or PNS.

The present invention may be used in conjunction with fetal, stem or other cell therapy, e.g. for neurological and psychiatric disorders or for the restoration or normalisation of damaged or abnormal tissue or function, in view of the neuroprotective and neurorestorative (neuroregenerative) effects of the active agents. Examples include cell therapy to treat brain disorders. The use of active agents in accordance with the present invention can improve the efficacy of the cell therapy, for example by increasing the survival rate of transplanted cells, by improving the efficiency of the surviving cells in the therapy, or a combination thereof.

The present invention may be used in a method of treatment of a disorder associated with abnormal expression of one or more NF or NFr in a human or non-human animal suffering from or susceptible to such a disorder. The disorders may be those disclosed in the prior art mentioned above, or may be different from those disorders. For example, the neurological methods may be autistic syndrome, depression and schizophrenia, or may be disorders other than these. Such disorders, other than neurological or psychiatric disorders or abnormal behavioural or personality traits, include for example the effects of sleep deprivation and stress, inflammatory disorders, allergies, immune disorders and NF-mediated cancers.

As mentioned above, the evidence in this application shows that the active agents used in the present invention can simultaneously normalise or enhance levels of both BDNF and GDNF in the brain. The present invention may therefore be used in a method of simultaneously normalising one or both of BDNF and GDNF levels in the brain of a human or non-human animal suffering from abnormal or reduced brain levels of one or both of those NFs.

The present invention provides a method of inducing self-regulated homeostasis of NFs such as BDNF and GDNF. The method is such that the induction of self-regulated homeostasis of NFs takes place with limited and manageable side effects. This application includes evidence that this induction does not require the presence of peptide NFs or NFrs. Therefore the invention avoids the need for co-administration of peptide NFs or NFrs with the non-peptide active agent(s) of the invention, in contrast to known agents.

Each of the new uses described above can be used with each of the aspects of the present invention.

The methods by which the invention is put into effect can be therapeutic or non-therapeutic and the compositions can be pharmaceutical or non-pharmaceutical compositions, as described in more detail below. The active agents are preferably orally administered, although other administration routes are provided for, as described in more detail below.

New Use (ii)—New Classes of Treatable Individuals

The new finding underlying the present invention reveals that the active agents can be used to treat individuals who may, at least at certain times, naturally overexpress or abnormally express one or more NFs or NFrs (e.g. BDNF and/or GDNF), for example sleep-deprived or stressed persons, whereas previously the treatment of such individuals by NF mimicking or stimulating agents was contraindicated.

The present invention may be used in a method of treating or preventing a disorder or condition associated with reduced or abnormal NF or NFr levels in a human or non-human animal suffering from or susceptible to such a disorder or condition, the said human or animal being an individual who is susceptible to naturally overexpress or abnormally express one or more other NFs or NFrs.

The new finding underlying the present invention also reveals that the active agents can be used to treat individuals who are susceptible to the psychiatric side effects of NF-mimicking or stimulating drugs, these side effects being typically psychiatric, mood, anxiety or other personality or behavioural symptoms, for whom previously the treatment by NF mimicking or stimulating agents was contraindicated.

The finding that the agents of the present invention work via self-regulated homeostasis of NFs, rather than modulation or binding to many receptors or enzymes, allows patients to be treated who are sensitive to adverse side-effects from some enzyme inhibiting drugs or receptor agonist drugs. For example, some dementia (Alzheimer's) patients cannot tolerate cholinesterase inhibitors. Some Parkinson's disease patients cannot tolerate L-dopa, and will suffer side-effects including dyskinesia or neuropsychiatric problems such as risk-taking.

The present invention may be used in a method of treating or preventing a disorder or condition associated with reduced or abnormal NF or NFr levels in a human or non-human animal suffering from or susceptible to such a disorder or condition, the said human or animal being an individual who is susceptible to the psychiatric or other side effects of NF-mimicking or stimulating drugs.

Disorders or conditions associated with reduced or abnormal NF or NFr levels include, for example, neurological, psychiatric, inflammatory, allergic, immune and neoplastic disorders or abnormal behavioral or personality traits, for example those described in more detail below. In addition, such disorders and conditions include the skin, muscle, eye and bone disorders and conditions described below, including conditions related to the wellbeing and health of the tissues and the condition of fatigue of the muscle or other tissue.

The new finding underlying the present invention also reveals that the active agents, which have no (ant)agonistic or binding capacity for a range of hormonal and other receptors and no enzyme binding capacity across a range of enzymes, can be used to treat individuals who are susceptible to receptor- or enzyme-mediated side effects of drugs. Such individuals may, for example, include individuals having an addiction or dependency, which may be exacerbated by an (ant)agonistic effect at a receptor which is influenced by the addiction or dependency; individuals who are in the process of treatment or self-treatment to be weaned off an addiction or dependency, where for the same reason the weaning-off process may be set back by an (ant)agonistic effect at a receptor which is influenced by the addiction or dependency; individuals with addictive or dependent personality types, for example having certain receptors or metabolic processes which are particularly sensitive to (ant)agonism or binding at receptors or enzyme binding. Furthermore, susceptibility to receptor- or enzyme-mediated side effects can arise in those undergoing treatments for other clinical conditions that would be interfered with by such receptor- or enzyme-mediated effects, for example individuals undergoing hormone treatment (e.g. hormone therapy in oncology, growth hormone treatment, thyroid hormone treatment, female hormone replacement therapy (HRT), or gender reassignment therapy).

The present invention may therefore be used in a method of treating or preventing a disorder or condition associated with reduced NF or NFr levels in a human or non-human animal suffering from or susceptible to such a disorder or condition, the said human or animal being an individual who is susceptible to receptor- or enzyme-mediated side effects of drugs.

The receptors or binding sites relevant to an individual's susceptibility to receptor (or binding site)-mediated side effects include any one or more of the following receptors: adensonine A₁ receptor; adensonine A_{2A} receptor; adensonine A₃ receptor; non-selective adrenergic α1 receptors, including adrenergic α_{1A}, adrenergic α_{1B}, or adrenergic α_{1D} receptor; non-selective adrenergic a2 receptors, including adrenergic α_{2A} or adrenergic α_{2C} receptor; non-selective adrenergic β receptors, including adrenergic β₁, adrenergic β₂ or adrenergic β₃ receptor; adrenomedullin AM₁ receptor; adrenomedullin AM₂ receptor; aldosterone receptor; anaphylatoxin C5a receptor; androgen (testosterone) receptor AR; angiotensin AT₁ receptor; angiotensin AT₂ receptor; apelin (APJ) receptor; atrial natriuretic factor receptor; bombesin BB1 receptor; bombesin BB2 receptor; bombesin BB3 receptor; bradykinin B₁ receptor; bradykinin B₂ receptor; calcitonin receptor; calcitonin gene-related peptide (CGRP₁) receptor; benzothiazepine L-type calcium channel; dihydropyridine L-type calcium channel; phenylalkylamine L-type calcium channel; calcium channel N-type; cannabinoid CB₁ receptor; cannabinoid CB₂ receptor; chemokine CCR1 receptor; chemokine CCR2B receptor; chemokine CCR4 receptor; chemokine CCR5 receptor; chemokine CXCR1 receptor; chemokine CXCR1 (IL-8R_{B}) receptor; cholecystokinin CCK₁ (CCK_{A}) receptor; cholecystokinin CCK₂ (CCK_{B}) receptor; colchicine receptor; corticotropin releasing factor (CRF₁) receptor; dopamine D₁ receptor; dopamine D_{2S} receptor; dopamine D₃ receptor; dopamine D_{4.2} receptor; dopamine D₅ receptor; endothelin ET_{A} receptor; endothelin ET_{B} receptor; epidermal growth factor (EGF) receptor; erythropoietin EPOR receptor; oestrogen receptors; oestrogen (ERα) receptor; oestrogen (ERβ) receptor; G protein-coupled receptor GPR103; G protein-coupled receptor GPR8; GABA_{A} receptor; TBOB chloride channel GABA_{A} receptor; central flunitrazepam GABA_{A} receptor; central muscimol GABA_{A} receptor; GABA_{B1A}; receptor; GABA_{B1B} receptor; gabapentin receptor; galanin GAL1 receptor; galanin GAL2 receptor; glucocorticoid receptor; glutamate receptors; AMPA glutamate receptor; kainate glutamate receptor; agonism NMDA glutamate receptor; glycine NMDA glutamate receptor; phencyclidine NMDA glutamate receptor; polyamine NMDA glutamate receptor; growth hormone secretagogue (GHS, Ghrelin) receptor; histamine H₁ receptor; histamine H₂ receptor; histamine H₃ receptor; histamine H₄ receptor; central imidazoline I₂ receptor; inositol triphosphate IP₃ receptor; insulin receptor; interleukin IL-1 receptor; interleukin IL-2 receptor; interleukin IL-6 receptor; leptin receptor; BLT leukotriene (LTB₄) receptor; cysteinyl leukotriene CysLT₁ receptor; cysteinyl leukotriene CysLT₂ receptor; melanocortin MC₁ receptor; melanocortin MC₃ receptor; melanocortin MC₄ receptor; melanocortin MC₅ receptor; melatonin MT₁ receptor; melatonin MT₂ receptor; motolin receptor; muscarinic M₁ receptor; muscarinic M₂ receptor muscarinic M₃ receptor; muscarinic M₄ receptor; muscarinic M₅ receptor; N-formyl peptide receptor FPR1; N-formyl peptide receptor-like FPRL1 receptor; neuromedin U MNU₁ receptor; neuromedin U MNU₂ receptor; neuropeptide Y Y₁ receptor; neuropeptide Y Y₂ receptor; neurotensin NT₁ receptor; nicotinic acetylcholine receptors; nicotinic acetylcholine α1, bungarotoxin receptor; nicotinic acetylcholine a7, bungarotoxin receptor; opiate δ (OP1, DOP) receptor; opiate κ (OP2, KOP) receptor; opiate μ (OP3, MOP) receptor; orphanin ORL₁ receptor; phorbol ester receptor; platelet activating factor (PAF) receptor; platelet-derived growth factor (PDGF) receptor; potassium channel [K_{A}]; potassium channel [K_{ATP}]; potassium channel [SK_{CA}]; potassium channel HERG; progesterone receptors; progesterone PR-B receptor; prostanoid CRTH2 receptor; prostanoid DP receptor; prostanoid EP₂ receptor; prostanoid EP₄ receptor; prostanoid thromboxane A₂ (TP) receptor; purinergic P_{2X} receptor; purinergic P_{2Y} receptor; retanoid X receptor RXRα; rolipram receptor; ryandine RyR3 receptor; serotonin 5-hydroxytryptamine 5-HT1 receptor; 5-hydroxytryptamine 5-HT_{1A} receptor; 5-hydroxytryptamine 5-HT_{1B} receptor; 5-hydroxytryptamine 5-HT_{2B} receptor; 5-hydroxytryptamine 5-HT_{2c} receptor; 5-hydroxytryptamine 5-HT₃ receptor; 5-hydroxytryptamine 5-HT₄ receptor; 5-hydroxytryptamine 5-HT_{5A} receptor; 5-hydroxytryptamine 5-HT₆ receptor; sigma σ₁ receptor; sigma σ₂ receptor; site 2 sodium channel receptor; somastatin sst1 receptor; somastatin sst2 receptor; somastatin sst3 receptor; somastatin sst4 receptor; somastatin sst5 receptor; tachykinin NK₁ receptor; tachykinin NK₂ receptor; tachykinin NK₃ receptor; testosterone receptor; thyroid hormone receptor; thyrotropin releasing hormone (TRH) receptor; transforming growth factor-β (TGF-β) receptor; adenosine transporter; choline transporter; dopamine transporter (DAT); GABA transporter; monoamine transporter; norepinephrine transporter (NET); 5-hydroxytryptamine transporter (SERT); non-selective tumour necrosis factor (TNF) receptor; urotensin II receptor; vanilloid receptor; vascular endothelial growth factor (VEGF) receptor; vasoactive intestinal peptide VIP₁ receptor; vasopressin V_{1A} receptor; vasopressin V_{1B} receptor; vasopressin V₂ receptor; and vitamin D₃ receptor.

The enzymes relevant to an individual's susceptibility to side effects include any one or more of the following enzymes: acetylcholinesterase; acetyl CoA synthetase; choline acetyltransferase; protein serine/threonine kinase AKT1 (PRKBA); protein serine/threonine kinase AKT3 (PRKBG); protein serine/threonine kinase CAMK2D (KCC2D); protein serine/threonine kinase MAP2K1 (MEK1); protein serine/threonine kinase MAPK1 (ERK2); protein serine/threonine kinase MAPK11 (p38β); protein serine/threonine kinase MAPK12 (p38γ); protein serine/threonine kinase MAPK13 (p38δ); protein serine/threonine kinase MAPK3 (ERK1); protein serine/threonine kinase MAPK8 (JNK1); non-selective protein serine/threonine kinase PKC; protein tyrosine kinase NTRK1 (trkA); protein tyrosine kinase NTRK2 (trkB); protein tyrosine kinase SRC; aldose reductase; ABTS radical free radical scavenger enzyme; DPPH radical free radical scavenger enzyme; SOD mimetic free radical scavenger enzyme; and UDP glucuronosyltransferase UGT1A1.

Therefore, the present invention has for the first time enabled small-molecule therapeutic agents for use on such individuals without side effects occurring from receptor- and enzyme-mediated activity of the active agents, or at least with a substantially reduced risk of such side effects occurring.

The inactivity of certain A/B-cis spirostane sapogenins and saponins at oestrogen, androgen, progesterone, glucocorticoid and testosterone receptors has been previously published in WO-A-99/48507, WO-A-99/48482, WO-A-01/23406, WO-A-01/23407, WO-A-01/23408 and WO-A-01/49703. The inactivity/non-binding of the same agents at muscarinic receptors was not shown although evidence of enhanced numbers and synthesis of muscarinic receptors was presented. Evidence of normalisation in numbers of muscarinic and adrenergic β2 receptors—without evidence relating to dose-dependency or activity/binding—was presented in WO-A-02/079221 and WO-A-03/082893.

Evidence of dose-dependent enhancement of the numbers of nicotinic receptors by a certain A/B-cis furostane saponin, timosaponin BII, has been previously published in WO-A-99/16786 (EP-A-1024146; U.S. Pat. No. 6,593,301). The extent of activity/binding of the agent at those receptors was apparently not measured. The further evidence presented now indicates that the effects reported in the prior art derive from the regulated increase in the synthesis or release, and/or reduction in the rate of degradation, of NFs and/or their receptors.

The present invention may be used in a method of treating or preventing neurodegeneration in a human or non-human animal in need thereof without inducing receptor- or enzyme-mediated side effects involving one or more of the receptors and enzymes listed from page 20, line 28 to page 23, line 7 above.

The methods can be therapeutic or non-therapeutic and the compositions can be pharmaceutical or non-pharmaceutical compositions, as described in more detail below. For example, a non-therapeutic use can be to improve neurological or psychological functioning of an individual within the normal range of the population. The terms “disorders”, “conditions” and “traits” will be understood accordingly. The active agents are preferably orally administered, although other administration routes are provided for, as described in more detail below.

New Use (iii)—New Combinations of Agents

The active agents in the present invention can be used in combination with other biologically active agents which are known or suspected to possibly cause an abnormal level of an NF or NFr in the subject (i.e. abnormally low or abnormally high levels), or may be used on a precautionary basis with one or more other biologically active agents for which a possibility of causing such abnormal levels is not known or suspected or has not been tested. Such other biologically active agents include active chemical agents such as pharmaceuticals, specific binding agents for inhibiting proteins or polynucleotides (for example, antibodies, antibody fragments such as F(ab) or F(ab)₂ fragments, siRNA or antisense DNA), and active tissues such as stem cells.

In this way, the agents according to the present invention can be used to counteract any potential adverse effects of the other biologically active agent(s).

The present invention may be used in a composition or set (collocated group) of compositions for administration to a human or non-human animal subject to treat or prevent a certain disorder or condition of the patient, the composition or set comprising a first bioactive agent for treating or preventing the said disorder or condition and having a potential to cause an abnormal level of an NF or NFr in the subject, and an active agent of the present invention for counteracting in a self-regulated manner any such abnormal NF or NFr level induced in the subject, whereby the said abnormal NF or NFr level is counteracted in the subject, preferably tending towards the normal NF or NFr level.

New Use (iv)—New Circumstances of Use

The present invention may be used in circumstances where close clinical control of an administration or dosing protocol is not available or practicable.

The resistance of the self-regulated treatment to over-dosing and the time-extended nature of the response combine to favour administration of the active agents under relatively poorly controlled circumstances, for example self-administration or non-therapeutic administration. The protocol for a self-regulated treatment method according to the present invention will be effective within a wider tolerance than corresponding prior art treatments.

Any of the methods using the present invention may therefore be applied in circumstances without clinical control of the administration protocol, particularly in circumstances of self-administration or non-therapeutic administration.

Any aspect of the present invention may be practised or used simultaneously with any one or more of the other aspects of the invention, and any example or preference stated for one aspect of the present invention shall apply equally to any other aspect of the invention.

### “Treating or Preventing”

The expression “treating or preventing” and analogous terms used herein refers to all forms of healthcare intended to remove or avoid the disorder or to relieve its symptoms, including preventive, curative and palliative care, as judged according to any of the tests available according to the prevailing medical and psychiatric practice. An intervention which aims with reasonable expectation to achieve a particular result but does not always do so is included within the expression “treating or preventing”. An intervention which succeeds in slowing or halting progression of a disorder is included within the expression “treating or preventing”.

Certain neurological, psychiatric, inflammatory, allergic and immune disorders are considered as “spectrum” conditions, in which individuals may exhibit some or all of a range of possible symptoms, or may exhibit only a mild form of the disorder. Furthermore, many neurological, psychiatric, inflammatory, allergic, immune and neoplastic conditions are progressive, starting with relatively mildly abnormal symptoms and progressing to more severely abnormal symptoms. The present invention includes the treatment and prevention of all NF-mediated neurological, psychiatric, inflammatory, allergic, immune and neoplastic conditions, of whatever type and stage

“Susceptible to”

The expression “susceptible to” and analogous terms used herein refers particularly to individuals at a higher than normal risk of developing a medical, health, wellbeing or psychiatric disorder, or a personality change, as assessed using the known risk factors for the individual or disorder. Such individuals may, for example, be categorised as having a substantial risk of developing one or more particular disorders or personality changes, to the extent that medication would be prescribed and/or special dietary, lifestyle or similar recommendations would be made to that individual.

### Toxicity and Side Effects

The agents according to the present invention have limited and manageable side effects and are non-toxic or essentially non-toxic in use.

In the context of pharmaceutical (including veterinary) use, this implies physiological acceptability of the agents, so that, within the scope of sound medical and veterinary judgement, the agents are suitable for use at an effective dosage in contact with cells of humans, mammals and other animals without undue toxicity, irritation, allergic response, undesirable side effects, and that such adverse events as may occur are deemed excessive or cannot be managed by side treatment, commensurate with a reasonable benefit/risk ratio.

In the context of functional foods, particularly foodstuffs, food supplements (including dietary supplements), beverages and beverage supplements, as well as topical preparations such as functional cosmetics and dermatological and other skin-contacting or eye-contacting preparations, this implies a corresponding assessment of benefit/risk and side effects, appropriate to the safety and toxicity standards for the particular composition or preparation and the particular use for which it is supplied.

### “Non-Therapeutic Method”

A non-therapeutic use is generally characterised by a human subject's elective self-administration, typically oral, of a physiologically active agent in a composition without medical supervision. Typically, the intended benefits from this will be wellbeing or general health benefits in relation to conditions or perceived conditions that are (i) formally undiagnosed, (ii) undiagnosable according to clinical practice, or (iii) within the normal ranges of the healthy population and therefore not considered as disorders.

A non-therapeutic use can also be characterised by the absence of medical intervention or assistance at the stage of the subject's purchasing or acquiring the composition.

Still further, a non-therapeutic use can be characterised by the absence of medical claims by the supplier of the composition, so that the self-administration is not driven by a specific intention to treat a diagnosed disorder.

For example, a neurological function that may suitably be influenced non-therapeutically may include, for example, cognition (including thinking, reasoning, memory, recall, imagining and learning), concentration and attention, particularly towards the milder end of the scale of conditions, and mild abnormal behavioural or personality traits. A psychological function that may suitably be treated non-therapeutically may include, for example, human behaviour, mood, personality and social function, for example sexual behaviour, sexual dysfunction, grief, anxiety, depression, moodiness, moroseness, teenage moods, disrupted sleep patterns, vivid dreaming, nightmares, and sleepwalking.

In addition to the examples of neurological and psychological functions given above that are treatable according to the non-therapeutic methods of the present invention, mild forms of neurological and psychiatric disorders, that are non-diagnosable according to clinical practice because the associated behaviours or thoughts do not cause significant distress to the individual or are not disruptive of his or her everyday functioning, may also be considered as conditions treatable non-therapeutically according to the present invention.

Mild forms of inflammatory, allergic and immune disorders, or inflammatory, allergic and immune disorders of unknown cause or which have for other reasons not received a formal diagnosis, may also be considered as conditions treatable non-therapeutically according to the present invention.

Benign neoplastic disorders, or neoplastic disorders of unknown cause or which have for other reasons not received a formal diagnosis, may also be considered as conditions treatable non-therapeutically according to the present invention.

### “Normalise”

The expression “normalise” and analogous terms (such as “homeostasis”) used herein refers particularly to a physiological adjustment towards a condition characteristic of general normal health. The optimum normal condition may be exemplified by the condition of a healthy young adult human or non-human animal.

“Normalise” thus includes the process of adjusting towards a normal condition, whether or not a condition is actually reached that would be characterised as normal.

### Neurological Disorders

The expression “neurological disorders” and analogous terms used herein includes, for example, neurodegeneration (including neurodegeneration with symptoms of impaired cognition and neurodegeneration without symptoms of impaired cognition), neuromuscular degeneration, and motor-sensory neurodegeneration.

Examples of neurological disorders with which the present invention is concerned include, without limitation: dementia, age-related cognitive impairment, Alzheimer's disease, senile dementia of the Alzheimer's type (SDAT), Lewy body dementia, vascular dementia, Parkinson's disease, postencephalitic Parkinsonism, parkinsonism having a cause other than postencephalitic and other than Parkinson's disease, muscular dystrophy including facioscapulohumeral muscular dystrophy (FSH), Duchenne muscular dystrophy, Becker muscular dystrophy and Bruce's muscular dystrophy, Fuchs' dystrophy, myotonic dystrophy, corneal dystrophy, reflex sympathetic dystrophy syndrome (RSDSA), neurovascular dystrophy, myasthenia gravis, Lambert Eaton disease, Huntington's disease, motor neurone diseases including amyotrophic lateral sclerosis (ALS), infantile spinal amyotrophy, multiple sclerosis, postural hypotension, pain, neuralgia, traumatic neurodegeneration e.g. following stroke or following an accident (for example, traumatic head or brain injury or spinal cord injury), Batten's disease, Cockayne syndrome, Down syndrome, corticobasal ganglionic degeneration, multiple system atrophy, cerebral atrophy, olivopontocerebellar atrophy, dentatorubral atrophy, pallidoluysian atrophy, spinobulbar atrophy, optic neuritis, sclerosing pan-encephalitis (SSPE), attention deficit disorder, post-viral encephalitis, post-poliomyelitis syndrome, Fahr's syndrome, Joubert syndrome, Guillain-Barre syndrome, lissencephaly, Moyamoya disease, neuronal migration disorders, autistic syndrome, polyglutamine disease, Niemann-Pick disease, progressive multifocal leukoencephalopathy, pseudotumor cerebri, Refsum disease, Zellweger syndrome, supranuclear palsy, Friedreich's ataxia, spinocerebellar ataxia type 2, Rhett syndrome, Shy-Drager syndrome, tuberous sclerosis, Pick's disease, chronic fatigue syndrome, neuropathies including hereditary neuropathy, diabetic neuropathy and mitotic neuropathy, prion-based neurodegeneration, including Creutzfeldt-Jakob disease (CJD), variant CJD, new variant CJD, bovine spongiform encephalopathy (BSE), GSS, FFI, kuru and Alper's syndrome, Joseph's disease, acute disseminated encephalomyelitis, arachnoiditis, vascular lesions of the central nervous system, loss of extremity neuronal function, Charcot-Marie-Tooth disease, Krabbe's disease, leukodystrophies, susceptibility to heart failure, asthma, epilepsy, auditory neurodegeneration, macular degeneration, pigmentary retinitis, and glaucoma-induced optic nerve degeneration.

### Psychiatric Disorders

The expression “psychiatric disorders” includes all human mental disorders which impact on personality and behaviour, and particularly in relation to a person's thinking, feeling, moods, and ability to relate to others. Thus there is some overlap between “neurological” and “psychiatric” disorders, and especially so in the present invention as the “psychiatric disorders” to be treated or prevented by the present invention will be directly or indirectly related to an underlying neurological defect which is directly or indirectly influenced by NFs or NFrs.

Generally speaking, mental disorders are not diagnosed as “psychiatric disorders” unless the associated behaviours or thoughts cause significant distress to the individual or are disruptive of his or her everyday functioning. There is therefore a borderline between diagnosable disorders and similar, but less severe or disruptive, psychological functions the treatment of which should be considered as non-therapeutic (see below).

Examples of psychiatric disorders with which the present invention is concerned include, without limitation: anxiety disorders (for example, acute stress disorder, panic disorder, agoraphobia, social phobia, specific phobia, obsessive-compulsive disorder, post-traumatic stress disorder, body dysmorphic disorder and generalized anxiety disorder), sexual anxiety disorders (for example, vaginismus, male erectile dysfunction, male orgasmic disorder and female orgasmic disorder), childhood disorders (for example, attention-deficit hyperactivity disorder (ADHD), Asperger's disorder, autistic disorder, conduct disorder, oppositional defiant disorder, separation anxiety disorder and Tourette's disorder), eating disorders (for example, anorexia nervosa and bulimia nervosa), mood disorders (for example, depression, major depressive disorder, bipolar disorder (manic depression), seasonal affective disorder (SAD), cyclothymic disorder and dysthymic disorder), sleeping disorders, cognitive psychiatric disorders (for example, delirium, amnestic disorders), personality disorders (for example, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive personality disorder), psychotic disorders (for example, schizophrenia, delusional disorder, brief psychotic disorder, schizophreniform disorder, schizoaffective disorder and shared psychotic disorder), and substance-related disorders (for example, alcohol dependence, amphetamine dependence, cannabis dependence, cocaine dependence, hallucinogen dependence, inhalant dependence, nicotine dependence, opioid dependence, phencyclidine dependence and sedative dependence).

### Inflammatory and Allergic Disorders

Examples of inflammatory and allergic disorders treatable according to the present invention include cough, pruritus (see Johansson, O et al, Arch. Dermatol. Res., 2002, 293, pages 614-619), food intolerance, psoriasis, croup, irritable bowel syndrome, tinnitus, Meniere's disease, stress-induced ulceration or acetylsalicylic acid-induced ulceration, allergic rhinitis, allergic dermatitis, conjunctivitis, inflammation, inflammatory bowel disease, ileitis, pancreatitis, cholecystitis, non-allergic rhinitis, oesophagitis, osteoarthritis, rheumatoid arthritis, hay fever, allergy to house mites, allergy to pet animals, Huntington's disease, acute inflammatory pain, visceral pain, dental pain and headaches, inflammatory hyperalgesia, tactile hyperalgesia (see, for example, Ma, Q P et al, Neuroreport 1997, 8, pages 807-810), allergic skin reactions, allergic eye reactions, asthma (see Bonini, S et al, Proc. Natl. Acad. Sci. USA, 1996, 93, pages 10955-10960; Braun, A et al, Am. J. Respiratory Cell Mol. Biol., 1999, 21, pages 537-546), atherosclerosis, arthritis, chonic ulcers (e.g. chronic vasulitic ulcers associated with rheumatoid arthritis) and eczema.

Related non-therapeutic treatments according to the present invention include to maintain normal breathing, to soothe sore throats and coughs, as an aid to maintain normal digestion, to ease upset stomachs, to aid in the recovery from colds and flu, as a decongestant, to soothe headaches, to relieve muscle soreness, to ease mild aches and pains, to provide relief from toothache, to provide relief from mouth or stomach ulcers, and to maintain healthy joints.

### Immune Disorders

Examples of NF-mediated immune disorders treatable according to the present invention include conditions which are treatable by normalisation of the action of NFs on the immune cell functions listed in Table 2 (page 8) of the Vega et al publication referenced above. Such disorders include immunodeficiency conditions such as AIDS (where the normalisation of NFs will boost the subject's immunocompetence), immune hyperactivity conditions (where the normalisation of NFs will down-regulate the subject's immune system), and conditions of impaired immune specificity (where the normalisation of NFs will assist the immune system to be more specific to foreign agents), for example autoimmune diseases such as systemic lupus erythematosus (SLE).

### Neoplastic Disorders

Examples of NF-mediated malignant neoplastic disorders treatable according to the present invention include cancer of the breast, thyroid, colon, lung, ovary, skin, muscle, pancreas, prostate, kidney, reproductive organs, blood, immune system (e.g. spleen, thymus and bone marrow), brain, peripheral nervous system and skin (e.g. melanoma and Kaposi's sarcoma).

### Restoration or Normalisation of Neuronal Function in, or in Relation to, Damaged or Abnormal Tissues

The present invention provides in one aspect restoration or normalisation of neuronal function in, or in relation to, damaged or abnormal tissues. The tissues can be brain tissues or tissues outside the brain, for example skin, bone, eye or muscle tissue.

This aspect of the invention may, for example, be used in connection with recovery of nerves after surgery, cuts, wounding, accidents, bruising, abrasions, burns, frostbite, bone fractures.

### Wound Healing

The present invention provides in one aspect assisting wounds to heal. The wounds can be any skin lesion, including chronic (e.g. ulcerous) skin lesions and acute skin lesions. The causes of such lesions are many and varied. Generally speaking all skin lesions are able to be treated beneficially using the present invention.

Aspects of wound healing that are measured to assess the quality of the healing include the rate of closure of the wound, the speed to regrowth of skin tissue over the wound, the colour of the healed wound in relation to the surrounding skin pigmentation, the mechanical strength of the healed wound in relation to the surrounding skin strength, the extent to which scar tissue or other skin tissue of abnormal texture or roughness remains on the wound after maximum healing, the time taken for the wound to cease exuding or for the exudate flow to ease, the physical appearance and smell of the wound or exudate, and the extent of pain, itching or other discomfort at various times in the healing process.

Against all these criteria, the present invention provides advantages in comparison with prior art treatments. The self-regulating homeostasis of the subject's native NFs, without necessarily the addition of exogenous NFs, will be expected to beneficially affect human and non-human mammalian skin lesions under all the criteria used.

The agents according to the present invention may be administered topically or systemically for the treatment of wounds. If administered topically, they may be delivered from any suitable composition or structure, for example a dressing for the wound or a cream or other preparation applied to the wound. Further details of delivery systems are provided below.

### Promoting or Assisting the Wellbeing and General Health of Tissues

The present invention provides in another aspect for promoting recovery of muscle and other tissues from exercise, exertion or wasting, and improving endurance and muscular stamina (e.g. in competitive or non-competitive sport) and reducing a feeling of fatigue. More generally, the wellbeing and general health of tissues, both in the brain and outside the brain, can be assisted according to the present invention.

In one example, cosmetic, eye or dermatological application of the agents according to the present invention to skin will improved the replenishment of new skin cells, and will thus assist a feeling of health and wellbeing of the skin or eyes. See, for example, Alber, K. M. et al, Neuroscientist 2007, 13, pages 371-382. The method according to the present invention, which involves self-regulated homeostasis of the skin NFs, avoids the administration of toxic agents to the body, and instead regulates the subject's own native NFs for the treatment.

These uses are generally, although not exclusively, non-therapeutic, being targeted in the main to healthy persons.

### Mammals

Besides being useful for human treatment, the present invention is also useful in a range of mammals, which can also be affected by neurological and psychological/psychiatric conditions. Such mammals include non-human primates (e.g. apes, monkeys and lemurs), for example in zoos, companion animals such as cats or dogs, working and sporting animals such as dogs, horses and ponies, farm animals, for example pigs, sheep, goats, deer, oxen and cattle, and laboratory animals such as rabbits or rodents (e.g. rats, mice, hamsters, gerbils or guinea pigs).

Where the disorder, condition, trait or function to be treated is exclusive to humans, then it will be understood that the mammal to be treated is a human. The same applies respectively to any other mammalian species if the disorder, condition, trait or function to be treated is exclusive to that species.

### Agents

The active agents used herein may generally, but not essentially, have a molecular weight less than about 800, for example less than about 700, for example less than about 600, for example less than about 500, for example less than about 450.

Following the standard nomenclature from steroid chemistry, the left hand 6-membered ring is named the A ring and the adjacent ring to the A-ring is named the B-ring Again following standard nomenclature from steroid chemistry, the carbon atoms are numbered as shown below, so that the line of fusion between the rings occurs between the 5- and 10-position carbon atoms.

In A/B-cis steroidal furostane/ene or spirostane/ene sapogenins, the substituent or hydrogen atom at both the 5- and the 10-position carbon atoms are orientated β to (above) the plane of the molecule.

This has the effect of kinking the plane of the molecule to create a pharmacophore group which looks as follows in a three-dimensional drawing. The substituent or hydrogen atom at the 10-position carbon atom is labelled as “a” in the drawing and the substituent or hydrogen atom at the 5-position carbon atom is labelled as “b”; the C ring is only partially shown):

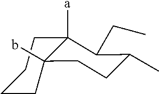

This is the A/B-cis motif

Examples of A/B-cis furostane/ene and spirostane/ene sapogenins and their derivative forms disclosed in WO-A-99/48482, WO-A-99/48507, WO-A-01/23407, WO-A-01/23408, WO-A-02/079221, WO-A-03/082893, WO-A-2005/105825 and WO-A-2006/048665 may be particularly mentioned as active agents for use in the present invention. The particular sets of compounds, and individual compounds, disclosed in these publications, representative of the class of compounds which is the A/B-cis furostane/ene and spirostane/ene sapogenins and ester, ether, ketone and glycosylated forms thereof, are incorporated herein by reference.

The ester, ether, ketone and glycoslyated forms of the A/B-cis furostane/ene and spirostane/ene sapogenins may be such that one or more ester, ether, ketone and glycoslyated group may be present in the molecule. Generally speaking, an ester, ether, ketone or glycoslyated group may be formed at any one or more OH moiety of the A/B-cis spirostane/ene sapogenin, using conventional chemical synthetic methods.

Examples of the active agents according to the present invention are the A/B-cis compounds represented by formula I in WO-A-01/23406 (page 6 of the published PCT application), formula II in WO-A-01/23406 (page 7 of the published PCT application), formula I in WO-A-01/23407 (page 6 of the published PCT application), formula II in WO-A-01/23407 (page 6 of the published PCT application), formula I in WO-A-01/23408 (page 6 of the published PCT application), formula I in WO-A-01/49703 (page 7 of the published PCT application), formula II in WO-A-02/079221 (page 6 of the published PCT application), formula I in WO-A-03/082893 (see page 4 of the published PCT application), formula II in WO-A-03/082893 (see page 4 of the published PCT application), formula III in WO-A-03/082893 (see page 5 of the published PCT application), formula I in EP-A-1024146 (see page 4 of the published EP application), and formula II in EP-A-102416 (see page 8 of the published EP application).

For example, the molecules sarsasapogenin and smilagenin and their corresponding ester, ether, ketone and saponin (glycosylated) derivatives are useful active agents for the present invention. The compound timosaponin BII, which is an A/B-cis furostane saponin, is a useful active agent for the present invention.

Other useful active agents for the present invention include episarsasapogenin, epismilagenin, metagenin, samogenin, diotigenin, isodiotigenin, texogenin, yonogenin, mexogenin and markogenin and their corresponding ester, ether, ketone and saponin derivatives.

The active agent may be used in any suitable crystalline or amorphous form, and in any suitable anhydrous, hydrated or solvated form. Further details of such forms of sarsasapogenin and smilagenin and their derivatives are given in WO-A-2005/105825 and WO-A-2006/048665, to which specific reference is directed.

The esters may especially include 3-position esters such as the carboxylate (e.g. cathylate (ethoxycarbonyloxy), acetate, succinate, cinnamate, ferulate, propionate, butyrate, isobutyrate, valerate, isovalerate, caproate, isocaproate, diethylacetate, octanoate, decanoate, laurate, myristate, palmitate, stearate, benzoate, phenylacetate, phenylpropionate, cinnamate, p-nitrobenzoyloxy, 3,5-dinitrobenzoyloxy, p-chlorobenzoyloxy, 2,4-dichlorobenzoyloxy, p-bromobenzoyloxy, m-bromobenzoyloxy, p-methoxybenzoyloxy, phthalyl, glycinate, alaninate, valinate, phenylalaninate, isoleucinate, methioninate, argininate, asparaginate, aspartate, cysteinate, glutamate, histidinate, lysinate, prolinate, serinate, threoninate, tryptophanate, tyrosinate, fumarate, maleate), phosphonate and sulphonate esters.

The ethers may especially include 3-position ethers such as the alkoxy derivatives (e.g. methoxy, ethoxy, n-propoxy, s-propoxy, n-butoxy, s-butoxy, t-butoxy).

The ketones (sapogenones) are typically the 3-keto derivatives of the corresponding sapogenins, although other keto derivatives formed at different OH-bearing carbon atoms of the ring system are also possible. Examples of 3-keto sapogenones include sarsasapogenone, smilagenone, episarsasapogenone and epismilagenone.

Examples of suitable saponin compounds include the compounds in which the carbon atom at the 3-position (i.e. the carbon to which R₃ is attached) carries in place of R₃ an O-sugar moiety, for example a mono-, di- or tri-saccharide or higher polysaccharide or an acylated form thereof. Examples of such sugar groups include sugar groups selected from glucose, mannose, fructose, galactose, maltose, cellobiose, sucrose, rhamnose, xylose, arabinose, fucose, quinovose, apiose, lactose, galactose-glucose, glucose-arabinose, fucose-glucose, rhamnose-glucose, glucose-glucose-glucose, glucose-rhamnose, mannose-glucose, glucose-(rhamnose)-glucose, glucose-(rhamnose)-rhamnose, glucose-(glucose)-glucose, galactose-(rhamnose)-galactose and acylated (e.g. acetylated) derivatives thereof.

Pseudosapo(ge)nins are ring-opened derivatives of the respective spirostane/ene sapogenins or saponins in which the F ring is opened and locked. Pseudosapo(ge)nins may have saturation or unsaturation at the C20-C22 bond. The saturated form is sometimes referred to as a “dihydropseudosapo(ge)nin” form.

The active agents for the present invention may be used singly or in any desired combination.

### Other Co-Agents or Co-Ingredients

The compositions used in the present invention may, if desired, include one or more co-agents and/or one or more co-ingredients, as described in more detail below in connection with the compositions and administration routes.

In particular, metabolic adjuvants, compounds that increase ketone body levels (ketogenic compounds), the tricarboxylic acid (TCA) cycle intermediates, compounds that are convertible in vivo to TCA intermediates, energy-enhancing compounds, or any mixture thereof may be used.

Metabolic adjuvants include vitamins (e.g. Vitamin E), minerals, antioxidants and other related compounds (for example, ascorbic acid, biotin, calcitriol, cobalamin, folic acid, niacin, pantothenic acid, pyridoxine, retinol, retinal (retinaldehyde), retinoic acid, riboflavin, thiamine, α-tocopherol, phytylmenaquinone, multiprenylmenaquinone, calcium, magnesium, sodium, aluminium, zinc, potassium, chromium, vanadium, selenium, phosphorus, manganese, iron, fluorine, copper, cobalt, molybdenum, iodine, or any combination thereof.

Ketogenic compounds generally enhance endogenous fat metabolism (oxidation) by the recipient and thereby raise the blood ketone levels, and include for example C₃₋₈ ketones such as acetone, D-β-hydroxybutyrate, metabolic precursors of D-β-hydroxybutyrate (for example acetoacetyl precursors such as acetoacetyl-1,3-butanediol, acetoacetyl-D-β-hydroxybutyrate and acetoacetylglycerol; esters such as esters of D-β-hydroxybutyrate with monohydric, dihydric or trihydric alcohols; or polyesters of D-β-hydroxybutyrate such as poly-D-β-hydroxybutyrate or terminally oxidised poly-D-β-hydroxybutyrate having from about 2 to about 100 repeats, e.g. from about 3 to about 10 repeats), metabolic precursors of acetoacetate, or any combination thereof.

TCA intermediates include citric acid, aconitic acid, isocitric acid, α-ketoglutaric acid, succinic acid, fumaric acid, malic acid, oxoacetic acid, or any combination thereof.

Compounds that are convertible in vivo to TCA intermediates include 2-keto-hydroxypropanol, 2,4-dihydroxybutanol, 2-keto-4-hydroxybutanol, 2,4-dihydroxybutyric acid, 2-keto-4-hydroxybutyric acid, aspartates, mono- and di-alkyl-oxaloacetates, pyruvate, glucose-6-phosphate, or any combination thereof.

Energy-enhancing compounds include, for example, Coenzyme CoQ-10, creatine, creatine derivatives, L-carnitine, n-acetyl-carnitine, L-carnitine derivatives, or any combination thereof. These compounds enhance energy production by a variety of means. Carnitine will increase the metabolism of fatty acids. CoQ-10 serves as an electron carrier during electron transport within the mitochondira. Accordingly, the addition of such compounds with active agents such as medium chain triglycerides (MCTs) will increase metabolic efficiency, especially in individuals who may be nutritionally deprived.

The co-agent, when present, may be provided in the form of a metabolic precursor such as a complex with one or more cations or as a salt, for use in therapy or nutrition. Examples of cations and typical physiological salts include sodium, potassium, magnesium, calcium salts, in each case the cation being balanced by a physiological counterion forming a salt complex such as L-lysine, L-arginine, methyl glucamine or others known in the art. The preparation and use of such metabolic precursors is described in WO-A-98/41201 and WO-A-00/15216, the disclosures of which are incorporated herein by reference.

### Compositions and Administration Routes

The active agent may be administered in the form of a composition comprising the active agent and any suitable additional component. The composition may, for example, be a pharmaceutical composition (medicament), a foodstuff, food supplement or beverage. Such a composition may contain a mixture of the specified compounds, and/or of their physiologically acceptable esters, amides, salts, solvates, analogs, or other suitable derivatives. In general, reference herein to the presence of one active agent and/or other component of a composition includes within its scope the presence of a mixture of two or more of such agents and/or components.

The pharmaceutical composition can be administered by any appropriate route including, but not limited to, oral, nasogastric, rectal, transdermal, parenteral (e.g. subcutaneous, intramuscular, intravenous, intramedullary and intradermal injections or infusions), intranasal, transmucosal, implantation, vaginal, topical, buccal and sublingual.

It is a typical feature of the use of a small-molecule somewhat lipophilic agent—as many of the active agents are—that the administration site can be remote from the brain of the mammal to be treated, the agent migrating through the bloodstream and crossing the blood-brain and/or blood-nerve barriers.

The term “pharmaceutical composition” in the context of this invention means a composition comprising an active agent and comprising additionally pharmaceutically acceptable carriers, diluents, adjuvants, excipients, or vehicles, such as preserving agents, fillers, disintegrating agents, buffering agents, preserving agents, penetration enhancers, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Suitable dosage forms include, for example, tablets, dragees, powders, elixirs, syrups, liquid preparations, including suspensions, sprays, inhalants, tablets, lozenges, emulsions, solutions, granules, capsules and suppositories, as well as liquid preparations for injections, including liposome preparations. Techniques and formulations generally may be found in Remington, Pharmaceutical Sciences, Mack Publishing Co., Easton, Pa., latest edition.

The terms “foodstuff”, “food supplement”, “beverage” and “beverage supplement” used herein have the normal meanings for those terms, and are not restricted to pharmaceutical preparations. These compositions are adapted for oral ingestion. Supplement compositions (e.g., a food supplement or beverage supplement) are arranged to be added to foods and beverages and ingested with them. A foodstuff typically may include calorific materials such as fats, oils and carbohydrates, as well as proteins and sources of minerals and fibre. Examples of compositions include dairy, cereal, vegetable, meat, fish, poultry or fruit based foodstuffs. Examples of beverages include carbonated and uncarbonated beverages, fruit juices, infusion drinks such as coffee or teas, for example herbal tea, fruit tea, Japanese green tea or Indian or Chinese tea. Compositions may comprise milk or milk-derived components, such as powdered milk and/or lactose and/or casein. The milk or milk-derived components are preferably derived from cows or goats. Plant-derived milks such as soya milk may be used. An edible composition may comprise one or more fermented components. The composition may comprise yogurt. Food supplements may, for example, contain vitamins, minerals, caffeine, ephedra alkaloids.

### Oral Compositions

Examples of suitable ingestible forms include, but are not limited to solid, dosage forms having a liquid, powder or solid core; chewable or oral disintegrating tablets; thin strips; gummi tablets; foam tablet; and coated particles having the salivation inducing agent in the coating and/or granulation matrix. In one embodiment, dosage forms are solid, semi-solid, or liquid compositions designed to contain a specific pre-determined amount (i.e. dose) of a certain ingredient, for example an active ingredient as defined below. Suitable dosage forms may be pharmaceutical drug delivery systems, including those for oral administration, buccal administration, or mucosal delivery; or compositions for delivering minerals, vitamins and other nutraceuticals, oral care agents, flavourants, and the like. In one embodiment, the dosage forms of the present invention may be considered to be solid; however, they may contain liquid or semi-solid components. In another embodiment, the dosage form is an orally administered system for delivering a pharmaceutical active ingredient to the gastro-intestinal tract of a human. Suitable co-agents in the composition may include analgesics, anti-inflammatory agents, antiarthritics, anesthetics, antihistamines, antitussives, antibiotics, anti-cancer agents, anti-allergic agents, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, immune system stimulants, decongestants, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof. Suitable oral care agents may be present, for example breath fresheners, tooth whiteners, antimicrobial agents, tooth mineralizers, tooth decay inhibitors, topical anesthetics, mucoprotectants, and the like. Suitable flavourants include menthol, peppermint, mint flavors, fruit flavors, chocolate, vanilla, bubblegum flavors, coffee flavors, liqueur flavors and combinations and the like. Examples of suitable gastrointestinal agents which may also be present include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine. Agents may also be present selected from analgesics, anti-inflammatories, and antipyretics: e.g. non-steroidal anti-inflammatory drugs (NSAIDs), including propionic acid derivatives: e.g. ibuprofen, naproxen, ketoprofen and the like; acetic acid derivatives: e.g. indomethacin, diclofenac, sulindac, tolmetin, and the like; fenamic acid derivatives: e.g. mefanamic acid, meclofenamic acid, flufenamic acid, and the like; biphenylcarbodylic acid derivatives: e.g. diflunisal, flufenisal, and the like; and oxicams: e.g. piroxicam, sudoxicam, isoxicam, meloxicam, and the like. In one embodiment, a coactive ingredient may be selected from propionic acid derivative NSAID: e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In another embodiment of the invention, the active ingredient may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment, a coactive agent may be selected from pseudoephedrine, phenylepherine, phenylpropanolamine, chlorpheniramine, dextromethorphan, diphenhydramine, guaifenesin, astemizole, terfenadine, fexofenadine, loratadine, desloratidine, doxilamine, norastemizole, cetirizine, benzocaine, mixtures thereof and pharmaceutically acceptable salts, esters, isomers, and mixtures thereof. In another embodiment, a coactive ingredient may be methylphenidate, modafinil and other active agents suitable for attention deficit hyperactivity disorder or attention deficit disorder; oxybutynin; sidenefil; and cyclobenzaprine. The active ingredient or ingredients are present in the dosage forms of the present invention in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. In one embodiment, the dosage form comprises at least about 85 weight percent of the active ingredient. The active ingredient or ingredients may be present in the dosage form in any form. For example, the active ingredient may be dispersed at the molecular level, e.g. melted or dissolved, within the dosage form, or may be in the form of particles, which in turn may be coated or uncoated. If the active ingredient is in form of particles, the particles (whether coated or uncoated) typically have an average particle size of about 1 micron to about 2000 microns. In one embodiment, such particles are crystals having an average particle size of about 1 micron to about 300 microns. In yet another embodiment, the particles are granules or pellets having an average particle size of about 50 microns to about 2000 microns, e.g. from about 50 microns to about 1000 microns or from about 100 microns to about 800 microns.

In one embodiment, oral compositions of the invention are food compositions, such as human or pet foods. In certain embodiments, the composition is a food composition, further comprising in addition to the active agent(s), about 15-50% protein, about 5-40% fat, about 15-60% carbohydrate, 5-10% ash content, each on a dry weight basis, and having a moisture content of about 5-20%. In certain embodiments, the foods are intended to supply complete necessary dietary requirements. Also provided are compositions that are useful as snacks, pet treats (e.g., biscuits), nutrition bars, and other forms for food products or dietary supplements, including tablets, capsules, gels, pastes, emulsions, caplets, and the like as discussed below. Optionally, the food compositions can be a dry composition (for example, kibble for pet food), semi-moist composition, wet composition, or any mixture thereof.

The compositions of the invention may be food products formulated specifically for human consumption. These will include foods and nutrients intended to supply necessary dietary requirements of a human being as well as other human dietary supplements. In a one embodiment, the food products formulated for human consumption are complete and nutritionally balanced, while in others they are intended as dietary supplements to be used in connection with a well-balanced or formulated diet.

The composition may be a food supplement, such as a gravy, drinking water, beverage, liquid concentrate, gel, yogurt, powder, granule, paste, suspension, chew, morsel, treat, snack, pellet, pill, capsule, tablet, or any other delivery form. The term “food supplement” includes dietary supplements. Dietary supplements can be specially formulated for consumption by a particular species or even an individual animal, such as companion animal, or a human. In one embodiment, the dietary supplement can comprise a relatively concentrated dose of the active agent(s) such that the supplement can be administered to the animal in small amounts, or can be diluted before administration to an animal. In some embodiments, the dietary supplement or other active-containing composition may require admixing with water or the like prior to administration to the animal, for example to adjust the dose, to make it more palatable, or to allow for more frequent administration in smaller doses.

The compositions of the present invention may be refrigerated or frozen. The active agent(s) may be pre-blended with the other components of the composition to provide the beneficial amounts needed, may be emulsified, coated onto a pet food composition, dietary supplement, or food product formulated for human consumption, or may be added to a composition prior to consuming it or offering it to an animal, for example, using a powder or a mix.

In one embodiment, the compositions comprise the active agent(s) in an amount effective to have the desired physiological or psychological or behavioural effect in an animal or human to which the composition has been administered. For pet foods and food products formulated for human consumption, the amount of active agent(s) as a percentage of the composition is in the range of about 1% to about 30% of the composition on a dry matter basis, although a lesser or greater percentage can be supplied. In various embodiments, the amount is about 1.0%, 1.5%, 2.0%, 2.5%, 3.0%, 3.5%, 4.0%, 4.5%, 5.0%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, or more, of the composition on a dry weight basis. Dietary supplements may be formulated to contain several fold higher concentrations of active agent(s), to be amenable for administration to an animal or human in the form of a tablet, capsule, liquid concentrated, or other similar dosage form, or to be diluted before administrations, such as by dilution in water, spraying or sprinkling onto a pet or human food, and other similar modes of administration. For a dietary supplement, the active agent(s) alone may be administered directly to the animal or human or applied directly to the animal's or human's regular food.

The compositions may optionally comprise supplementary substances such as minerals, vitamins, salts, condiments, colorants, and preservatives. Non-limiting examples of supplementary minerals include calcium, phosphorous, potassium, sodium, iron, chloride, boron, copper, zinc, magnesium, manganese, iodine, selenium, and the like. Non-limiting examples of supplementary vitamins include vitamin A, any of the B vitamins, vitamin C, vitamin D, vitamin E, and vitamin K, including various salts, esters, or other derivatives of the foregoing. Additional dietary supplements may also be included, for example, any form of niacin, pantothenic acid, inulin, folic acid, biotin, amino acids, and the like, as well as salts and derivatives thereof. In addition, the compositions may comprise beneficial long chain polyunsaturated fatty acids such as the (n-3) and/or (n-6) fatty acids, arachidonic acid, eicosapentaenoic acid, docosapentaenoic acid, and docosahexaenoic acid, as well as all combinations thereof.

The compositions provided herein optionally comprise one or more supplementary substances that promote or sustain general neurologic health, or further enhance cognitive function. Such substances include, for example, choline, phosphatidylserine, alpha-lipoic acid, CoQ10, acetyl-L-carnitine, and herbal components or extracts containing for example, one or more components from such plants as Ginko biloba, Bacopa monniera, Convolvulus pluricaulis, and/or Leucojum aestivum.

In various embodiments, the foodstuff or food/dietary supplement compositions provided herein preferably comprise, on a dry weight basis, from about 15% to about 50% crude protein. The crude protein material comprise one or more proteins from any source whether animal, plant, or other. For example, vegetable proteins such as soybean, cottonseed, and peanut are suitable for use herein Animal and dairy proteins such as casein, albumin, and meat protein, including pork, lamb, equine, poultry, fish, or mixtures thereof are useful.

The compositions may further comprise, on a dry weight basis, from about 5% to about 40% fat. The compositions may further comprise a source of carbohydrate. The compositions typically comprise from about 15% to about 60% carbohydrate, on a dry weight basis. Examples of such carbohydrates include grains or cereals such as rice, corn, sorghum, alfalfa, barley, soybeans, canola, oats, wheat, or mixtures thereof. The compositions also optionally comprise other components that comprise carbohydrates such as dried whey and other dairy products or by-products.

The compositions may also comprise at least one fibre source. Any of a variety of soluble or insoluble fibres suitable for use in foods or feeds may be utilised, and such will be known to those of ordinary skill in the art. Suitable fibre sources include beet pulp (from sugar beet), gum arabic, gum talha, psyllium, rice bran, carob bean gum, citrus pulp, pectin, fructooligosaccharide additional to the short chain oligofructose, mannanoligofructose, soy fibre, arabinogalactan, galactooligosaccharide, arabinoxylan, or mixtures thereof. Alternatively, the fibre source can be a fermentable fibre. Fermentable fibre has previously been described to provide a benefit to the immune system of a companion animal. Fermentable fibre or other compositions known to those of skill in the art which provide a prebiotic composition to enhance the growth of probiotic microorganisms within the intestine may also be incorporated into the composition to aid in the enhancement of the benefit provided by the present invention to the immune system of an animal. Additionally, probiotic microorganisms, such as Lactobacillus or Bifidobacterium species, for example, may be added to the composition.

In another embodiment oral compositions of the present invention are carbonated beverage compositions, including concentrates therefor. Such compositions may be prepared by methods which are well known in the art.

In this embodiment, by virtue of carbon dioxide (forming carbonic acid in water) the beverage is normally acidic. However, it is possible for such a beverage to be “acidulated”, i.e. adjusted so that it contains an additional acid of the type to be found in a “tangy” beverage. Examples may include phosphoric acid, and food acids (sometimes called “wholesome acids”) such as citric acid, maleic acid, fumaric acid and tartaric acid. Fruit, fruit juices and fruit extracts contain food acids, so beverages containing these components may be considered as acidulated.

The beverage may be non-alcoholic. Examples include cola drinks, orange drinks, lemon drinks, lemonade, tonic water, root beer, ginger ale and ginger beer.

The beverage may be alcoholic, typically having 3-9% wt/wt ethanol. Examples include cider and so-called “alcopops”, which are often carbonated blends of vodka or other spirits, with fruit flavourings. The beverage may be lightly alcoholic, typically having 0.1-3% wt/wt ethanol. Examples include shandy and certain fermented types of root beer, ginger beer and lemonade.

The carbonated beverage may be a non-dairy product, for example a milk-free or yoghurt-free beverage. The carbonated beverage may be substantially fat-free.

The beverage may be a flavoured water based beverage.

The carbonated beverage may be clear or cloudy or turbid or opaque.

The carbonated beverage may contain vitamins, for example one or more of A, B, C, D, E and K group vitamins. Vitamins may be added in addition to vitamins present in other components, such as fruit juice. Water-soluble vitamins B and C are very suitable components of the beverage. Fat soluble vitamins A, D, E and K are less so. Preferably vitamin E or derivatives thereof are not present in the beverage. Preferably vitamins A and K, or derivatives thereof, are not present in the beverage.

The carbonated beverage may contain a sweetening agent. The sweetening agent may be a natural or synthetic sweetening agent, for example sugar, corn syrup, sugar alcohol (for example sorbitol, xylitol, mannitol, maltitol or isomalt), or an intense sweetener (for example saccharin, sucralose, neotame, acesulfame potassium or aspartame), or any combination thereof.

### Topical Compositions

In another embodiment the compositions of the present invention are topical compositions, for example cosmetic, eye or dermatological compositions.

Topical compositions for delivery of the active agent are formulated in any suitable way. The topical compositions may be formulated into wound dressings or other mechanical application systems in conventional way.

The active agent compounds described herein can be prepared and delivered together with one or more cosmetically and/or dermatologically acceptable carriers therefore, and optionally, other therapeutic ingredients. Carriers should be acceptable in that they are compatible with any other ingredients of the composition and not harmful to the recipient thereof. A carrier may also reduce any undesirable side effects of the agent. Such carriers or vehicle ingredients are known in the art. See, Handbook of Cosmetic Science and Technology Taylor & Francis Group, 2006, herein incorporated by reference in its entirety.

Composition for topical administration according to the invention can be for local and/or systemic use, depending upon the active ingredient provided therein and the area and frequency of administration. Thus, the following discussion directed to topical formulations could be viewed as describing systemic formulations to the extent an active agent capable of topical systemic administration is included therein.

Compositions for topical administration used in the combinations of the invention can be incorporated into any pharmaceutical, cosmetic, eye or dermatological preparation customarily used and which may exist in a variety of forms. For example, the composition for topical administration may be a solution, a water-in-oil (W/O) type emulsion, an oil-in-water (O/W) type emulsion, or a multiple emulsion, for example a water-in-oil-in-water (W/O/W) or oil-in-water-in oil (O/W/O) emulsion, a hydrodispersion or lipodispersion, a gel, a cream, a solid stick, or an aerosol. Emulsions in accordance with the present invention, for example in the form of a cream, a lotion or a cosmetic milk, are advantageous and comprise, for example, fats, oils, waxes and/or other lipids, as well as water and one or more emulsifiers as they are usually used for such a type of formulation.

In certain embodiments, compositions for topical administration according to the invention may be used, for example, as a protective skin cream, cleansing milk, sun protection lotion, nutrient cream, day cream or night cream and the like, depending on their composition.

The compositions for topical administration may comprise cosmetically active ingredients, cosmetic auxiliaries and/or cosmetic additives conventionally used in such preparations. These include, for example, antioxidents, preservatives, bactericides, thickeners, fillers, antifoams, fragrances, essential oils, pigments (e.g. fumed silica, microfine pigments such as oxides and silicates including optionally coated iron oxide, titanium dioxide, boron nitride, and barium sulfate), ceramides (either as natural materials or functional mimics of natural ceramides), surfactants, emulsifiers, phospholipids, cholesterol, phytosphingosines, additional active ingredients such as vitamins or proteins (e.g. retinyl palmitate or acetate, Vitamin B as panthenol and its derivatives, Vitamin E as tocopheryl acetate, Vitamin F as polyunsaturated fatty acid esters such as such as gamma-linolenic acid esters), sunscreens (including chemical sunscreens and dispersed physical sunscreens), stabilizers, insect repellents, alcohols, plasticizers, polyols, polymers, foam stabilizers, electrolytes, organic solvents, silicone derivatives, moisturizers and/or humectants, fats, oils, waxes, water, salts, proteolytically or keratolytically active substances, and the like. Such additives can be present in dermatological or cosmetic compositions for topical administration.

As noted above, in addition to the active agent for topical delivery, the topical compositions of the invention can also comprise one or more additional active agents or materials providing a beneficial effect. For example, in specific embodiments, the topical compositions can comprise a sun protection product. These preferably comprise, in addition to the active ingredient used in accordance with the invention, at least on additional UVA filter and/or at least one UVB filter and/or at least one inorganic pigment.

The UVB filters may be soluble in oil or in water. Examples of substances which are soluble in oil are, for example: 3-benzylidenecamphor and its derivatives, for example 3-(4-methylbenzylidene)camphor, 4-aminobenzoic acid derivatives, preferably 2-ethylhexyl 4-dimethylaminobenzoate, amyl 4-dimethylaminobenzoate; cinnamic esters, preferably 2-ethylhexyl 4-methoxycinnamate, isopentyl 4-methoxycinnamate; salicylic esters, preferably 2-ethylhexyl salicylate, 4-isopropybenzyl salicylate, homomethyl salicylate; benzophenone derivatives, preferably 2-hydroxy-4-methoxybenezophenone, 2-hydroxy-4-methoxy-4′-menthylbenzophenone, 2,2′-dihydroxy-4-methoxybenzophenone; benzalmalonic esters, preferably di(2-ethylhexyl)4-methoxybenzalmalonate; 2,4,6-trianillino-(p-carbo-2′-ethyl-1′-hexyloxy)-1,3,5-trizane.

Advantageous substances which are soluble in water are: 2-phenylbenzimidazole-5-sulphone acid and its salts, for example sodium, potassium or triethanolammonium salts, sulphonic acid derivatives of benzophenones, preferably 2-hydroxy-4-methoxybenzophenone-5-sulphonic acid and its salts; sulphonic acid derivatives of 3-benzylidenecamphor such as, for example, 4-(2-oxo-3-bornylidene-methyl)benzenesulphonic acid, 2-methyl-5-(2-oxo-3-bornylidenemethyl)sulphonic acid and their salts. Naturally, the list of the abovementioned UVB filters which may be used according to the invention is not intended to be limiting.

Examples of UVA filters than can be used according to the invention include dibenzoylmethane derivatives, in particular 1-(4′-tert-butylphenyl)-3-(4′-methoxyphenyl)propane-1,3-dione and 1-phenyl-3-(4′-isopropylphenyl)propane-1,3-dione.

Examples of inorganic pigments that can be used according to the invention include oxides of titanium, zinc, iron, zirconium, silicon, manganese, aluminum, cerium and mixtures of these, and modifications where the oxides are the active agents. Especially preferably, they are pigments based on titanium dioxide.

Advantageous antioxidants which may be used in accordance with the invention are all those antioxidants which are suitable or conventional for cosmetic and/or eye and/or dermatological applications. The antioxidants are advantageously selected from the group consisting of amino acids (e.g. glycine, histidine, tyrosine, tryptophan) and their derivatives, imidazoles (e.g. urocaninic acid) and their derivatives, peptides such as D,L0carnosine, D-carnosine, L-carnosine and their derivatives (e.g. anserine), carotenoids, carotenes (e.g. alpha-carotene, beta-carotene, lycophene) and their derivatives, aurothioglucose, propylthiouracil and other thiols (e,g, thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionicacid and its derivatives (e.g. esters, ethers, peptides, lipids, nucleotides, nucleosides and salts) and sulphoxime compounds (e.g. buthionine sulphoximines, homocysteine sulphoximine, buthionine sulphones, penta-, hexa-, heptathionine sulphoximine) at very low tolerated doses (e.g. pmol to μmol/kg), furthermore (metal)chelating agents (e.g. alpha-hydroxy fatty acids, palmitic acid, phytic acid, lactoferrin), alpha-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. gamma-linolenic acid, linolic acid, oleic acid), folic acid and its derivatives, alaninediacetic acid, flavonoids, polphenols, catechols, ubiquinone and ubiquinol and their derivatives, vitamin C and derivatives (e.g. ascorbyl palmitate, Mg-ascorbyl phosphate, ascorbyl acetate), tocopherols and derivatives (e.g. vitamin E acetate), and coniferyl benzoate of benzoin resin, rutinic acid and its derivatives, ferulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (e.g. ZnO, ZnSO₄) selenium and its derivatives (e.g. selenium methionine), stilbene and its derivatives (e.g. stilbene oxide, trans-stilbene oxide) and those derivatives of the abovementioned active ingredients which are suitable according to the invention (e.g. salts, esters, ethers, sugars, nucleotides, nucleosides, peptides and lipids).

When provided as solution, emulsion, or dispersion, the compositions for topical administration can comprise solvents exemplified by the following: water or aqueous solutions; oils such as triglycerides of capric or caprylic acid, preferably castor oil; fats, waxes and other natural and synthetic lipids, preferably esters of fatty acids with alcohols of low C number, for example with isopropanol, propylene glycol or glycerol, or esters of fatty alcohols with alkanolic acids of low C number or with fatty accords; alcohols, diols or polyols of low C number and their ethers, preferably ethanol, isopropanol, propylene glycol, glycerol, ethylene glycol, ethylene glycol monoethyl ether or ethylene glycol monobutyl ether, propylene glycol monomethyl ether, propylene glycol monoethyl ether or propylene glycol monobutyl ether, diethylene glycol monomethyl ether or diethylene glycol monoethyl ether, and analogues products. Moreover, mixtures of the above-mentioned solvents can be used. In particular reference to alcoholic solvents, water may be a further constituent.

The oil phase of the emulsions, oleogels or hydro- or lipodispersions in accordance with the present invention is advantageously selected from the group of the esters of saturated and/or unsaturated, branches and/or unbranched alkanecarboxylic acids with a chain length of 3 to 30 C atoms and saturated and/or unsaturated branched and/or unbranched alcohols with a chain length of 3 to 30 C atoms, from the group of esters of aromatic carboxylic acids and saturated and/or unsaturated, branched and/or unbranched alcohols with a chain length of 3 to 3 C atoms. In this case, such ester oils may be selected advantageously from the group consisting of isopropyl myristate, isopropyl palmitate, isopropyl stearate, isopropyl oleate, n-butyl stearate, n-hexyl laurate, n-decyl oleate, isoctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, for example jojoba oil.

Furthermore, the oil phase may advantageously be selected from the group of the branched and unbranched hydrocarbons and hydrocarbon waxes, the silicone oils, the dialkyl ethers, the group of the saturated or unsaturated branched or unbranched alcohols and of the fatty acid triglycerides, viz, the triglycerol esters of saturated and/or unsaturated, branched and/or unbranched alkanecarboxylic acids with a chain length of 8 to 24, in particular 12-18, C atoms. For example, the fatty acid triglycerides may advantageously be selected from the group of the synthetic, semisynthetic and natural oils, for example olive oil, sunflower oil, soya oil, peanut oil, rapeseed oil, almond oil, palm oil, coconut oil, palm kernel oil, and the like. Any mixtures of such oil and wax components may also advantageously be employed in accordance with the present invention. If appropriate, it may also be advantageous to employ waxes, for example cetyl palmitate, as the only lipid component of the oil phase.

The oil phase is advantageously selected from the group consisting of 2-ethylhexyl isostearate, octyldodecanol, isotridecyl isononanoate, isoeicosan, 2-ethylhexyl cocoate, C12-15-alkyl benzoate, caprylic/capric acod triglyceride, dicaprylyl ether. Especially advantageous mixtures are those of C12-15-alkyl benzoate and 2-ethylhexyl isostearate, those of C12-15-alkyl benzoate and isotridecyl isononanoate and those of C12-15-alkyl benzoate, 2-ethylhexyl isostearate and isotridecyl isononanoate. In relation to hydrocarbons, liquid paraffin, squalane and squalene may advantageously be used according to the present invention. The oil phase may furthermore advantageously comprise cyclic or linear silicone oils, or consist entirely of such oils, but it is preferred to use an additional content of another oil phase components, apart from the silicone oil(s). Cyclomethicone (octamethylcyclotetrasiloxane) is advantageously employed as silicone oil to be used according to the invention. However, other silicone oils may be used advantageously in accordance with the present invention, for example hexamethylcyclotrisiloxane, polydimethylsiloxane, poly(methylphenylsiloxane). Especially advantageous mixtures are furthermore those of cyclomethicone and isotridecyl isononanoate and of cyclomethicone and 2-ethylhexyl isostearate.

If appropriate, the aqueous phase of the preparations according to the invention advantageously comprises alcohols, diols or polyols of low C number, and their ethers, preferably ethanol, isopropanol, propylene glycol, glycerol, ethylene glycol, ethylene glycol monoethyl ether or ethylene glycol monobutyl ether, propylene glycol monomethyl ether, propylene glycol monoethyl ether or propylene glycol monobutyl ether, diethylene glycol monomethyl ether or diethylene glycol monoethyl ether and analogous products, furthermore alcohols of low C number, for example ethanol, isopropanol, 1,2-propanediol, glycerol, and, in particular, one or more thickeners which may advantageously be selected from the group consisting of silicon dioxide, aluminum silicates, polysaccharides and their derivatives, for example hyaluronic acid, xanthan gum, hydroxypropylmethylcellulose, especially advantageously from the group of the polyacrylates, preferably a polyacrylate from the group of the so-called Carbopols, for example type 980, 981, 1382, 2984 and 5984 Carbopols, in each case singly or in combination.

Gels used according to the invention usually compromise alcohols of low C number, for example ethanol, isoproponal, 1,2-propanediol, glycerol and water, or an above-mentioned oil in the presence of a thickener, which is preferably silicon dioxide or an aluminum silicate in the case of oily-alcoholic gels and preferably a polyacrylate in the case of aqueous-alcoholic or alcoholic gels.

Solid sticks comprise, for example, natural or synthetic waxes, fatty alcohols or fatty acid esters. Customary basic materials which are suitable for use as cosmetic sticks in accordance with the present invention are liquid oils (for example liquid paraffin, castor oil, isopropyl myristate), semi-solid constituents (for example petrolatum, lanolin), solid constituents (for example beeswax, ceresine and micro-crystalline waxes, or ozocerite) and waxes of high melting point (for example carnauba wax, candelilla wax).

Suitable propellants for cosmetic and/or dermatological preparations in accordance with the present invention which can be sprayed from aerosol containers are the customary known volatile, liquefied propellants, for example hydrocarbons (propane, butane, isobutene), which may be employed singly pr as a mixture with each other. Pressurized air may also be used advantageously. The person skilled in the art will, of course, be familiar with the fact that there are non-toxic propellants, which would be suitable in principle for putting into practice the present invention in the form of aerosol preparations; however, it is recommended to manage without these-in particular fluorohydrocarbons and fluorochlorohydrocarbons (FCHCs)-due to their unacceptable effect on the environment or other accompanying circumstances.

Compositions for topical administration in accordance with the present can also be in the form of gels comprising not only an effective amount of active ingredient according to the invention and conventionally used solvents therefore, but also organic thickeners. Example of such thickeners include gum Arabic, xanthan gum, sodium alginate, cellulose derivatives, preferably methylcellulose, hydroxymethylcellulose, hydroxyethylcellulose, hydroxypropylcellulose, hydroxypropylmethylcellulose, or inorganic thickeners, for example aluminum silicates such as, for example, bentonites, or a mixture of polyethylene glycol and polyethylene glycol stearate or polyethylene glycol distearate.

An example of an acceptable cosmetic/dermatological carrier formulation containing the above-noted active ingredients can include the following ingredients: Xanthan Gum; Glycerin 99.7%; Tetrasodium EDTA; Glyceryl Stearate and PEG-100 Stearate (ARLACEL™ 165); Cetyl Alcohol; Isopropyl Palmitate; Butylated hydroxytoluene (BHT); Methylparaben; Propylparaben; and Deionized Water. Another example of an acceptable cosmetic/dermatological carrier formulation containing the above-noted active ingredients can include the following inert ingredients: Steric Acid; Cetyl Alcohol; Laureth 4; CARSONOL™ Sles; Propyl Paraben; Ascorbyl Palmitate; Propylene Glycol; CARBOPOL™ 974 P; Methyl Paraben; KOH (10%); and H2O.

The above noted composition can be prepared by a process, for example, as follows:
1. Combine and melt oil phase: Stearic acid, Cetyl Alcohol, Laureth 4, Propyl Paraben and Ascorbyl Palmitate;
2. In a glass beaker, combine Propylene Glycol and water, disperse Methyl Paraben and CARBOPOL™ with high-speed propeller stirring;
3. Add CARSONOL™ Sles to product of Step (2);
4. Warm product of Step (3) to 65-70° C.;
5. With mixing, add product of Step (1) to product of Step (4) and mix well;
6. Cool mixture to 40° C.;
7. Add solvent portion and mix well by hand;
8. Add KOH solution to neutralize; and
9. Protect from light.

### GENERAL

In each case, the composition may suitably contain one or more other active agents, which may be selected from the A/B-cis spirostane or spirostene steroidal sapogenins and ester, ether, ketone and gluycosylated forms thereof, other sapo(ge)nins, other non-sapo(ge)nin active agents, or any combination thereof. The composition may contain one or more biologically inert ingredients, for example diluents, carriers and excipients, which serve purposes related to presentation, administration or delivery of the physiologically active component, or which provide associated benefits to the subject separately from the physiological effects of the active component. The carriers may comprise plant materials such as soya protein. The composition may, for example, also comprise any one or more of preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The composition for use in the present invention, particularly the pharmaceutical composition, may be in unit dosage form, whereby a certain number of such forms is administered to the subject in a certain time period, according to the condition to be treated or prevented. Alternatively, the composition may be in bulk form, whereby a certain weight or volume of the bulk composition is measured out and administered to the subject in a certain time period, according to the condition to be treated or prevented.

However, toxicity is not considered to be a problem with these active agents, even at the higher dosages. The selection of appropriate dosages is thus within the ability of one of ordinary skill in this art, without undue burden. The administered dosage of the active than about 0.3 mg/kg body weight, preferably administered once per day. More typically, the dosage will be between about 0.1 and about 25 mg/kg, e.g. between about 1 and about 10 mg/kg, preferably administered once or twice per day. For adult human use, the dosage may conveniently be between about 10 and about 700 mg per day.

The composition for use in the present invention may suitably contain other therapeutic and/or non-therapeutic bioactive agents, as discussed above.

The composition for use in the present invention may be in unit dosage form, whereby a certain number of such forms is administered to the subject in a certain time period, according to the condition to be treated or prevented. Alternatively, the composition may be in bulk form, whereby a certain weight or volume of the bulk composition is measured out and administered to the subject in a certain time period, according to the condition to be treated or prevented.

The required dosage of the active agent will vary widely, depending on the severity of the symptoms to be treated or prevented. A concentration in the femtomolar to micromolar range is effective, for example about 1 fM to about 5 μM. The experimental work reported in Example 12 shows an in vitro EC₅₀ 13.4 fM of smilagenin against neuronal damage in culture. In general, a blood plasma concentration in vivo in the picomolar to micromolar range (for example in the nanomolar to micromolar range) is generally preferred, for example above about 1 pM, for example in the range of about 1 pM to about 5 μM, for example about 1 pM to about 3 μM, for example about 10 pM to about 700 nM, for example about 0.1 nM to about 500 nM. Below picomolar, the in vivo activity of the active agents tends to decline. Above micromolar, the self-regulation and the associated resistance of the subject to overdosing will simply mean that the active agent is wasted. However, as the examples in this application show, toxicity is not considered to be a problem with these active agents, even at the higher dosages. The selection of appropriate dosages is thus within the ability of one of ordinary skill in this art, without undue burden. The administered dosage of the active agent may, for example, be greater than about 0.1 mg/kg body weight, for example greater than about 0.3 mg/kg body weight, preferably administered once per day. More typically, the dosage will be between about 0.1 and about 25 mg/kg, e.g. between about 1 and about 10 mg/kg, preferably administered once per day. For adult human use, the dosage may conveniently be between about 10 and about 700 mg per day.

For further details of suitable composition forms and dosages, and examples of conditions and diseases treatable according to the present invention, please refer to WO-A-99/48482, WO-A-99/48507, WO-A-01/23407, WO-A-01/23408, WO-A-02/079221, WO-A-03/082893, WO-A-2005/105825 and WO-A-2006/048665.

The active agents are suitably formulated with one or more carrier, excipient and/or diluent in the composition. Generally speaking, any conventional carrier, excipient and/or diluent used for pharmaceutical compositions, oral compositions such as foodstuffs, food supplements and beverages, or topical compositions such as cosmetic, eye or skin preparations may be used.

Many of the active agents are relatively lipophilic, and in this case solubilising and/or suspending and/or dispersing agents may suitably be used to maintain the active agent in solution or suspension or dispersion in the composition.

Two group of solubilising and/or suspending and/or dispersing agents that may particularly be mentioned are the MCTs and the medium chain fatty acids (MCFAs). These are lipophilic compounds having fatty acid chains with chain lengths of between about 4 and about 12 carbon atoms.

Preferred examples of MCTs are represented by the following general formula (I):

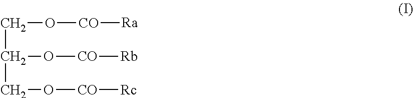

wherein Ra, Rb and Rc are, independently of each other, selected from saturated or unsaturated fatty acid residues having 4 to 12 carbon atoms in the carbon backbone.

Preferred examples of MCFAs are represented by the following general formula (II):

HO—CO—Rd (II)

wherein Rd is a saturated or unsaturated fatty acid residue having from 4 to 12 carbon atoms in the carbon backbone.

Examples of Ra, Rb, Rc and Rd include residues of caproic (C6:0), caprylic (C8:0), capric (C10:0) and lauric (C12:0) acids. In the standard naming system, the number immediately after the letter C indicates the carbon chain length and the number immediately after the colon (:) indicates the number of unsaturated bonds. Such MCTs and MCFAs can be obtained in known manner from natural sources such as coconut oil, palm kernel oil and camphor drupes (fruits). The residues of one or more than one fatty acids may be present in a commerical MCT or MCFA product.

MCTs for use in the present invention may, for example, be selected from tri-C6:0 MCT, tri-C8:0 MCT and tri-C10:0 MCT.

### INDUSTRIAL APPLICABILITY AND UTILITY

The present invention makes available for the first time self-regulated methods of therapeutic and non-therapeutic treatment of NF-mediated disorders and functions in human and non-human mammals, in which the physiological response is not dose-dependent but self-regulates within a relatively wide range of dosages of the active agent(s), while providing a relatively narrow “therapeutic window” in terms of predictable beneficial physiological effects without adverse side effects or toxicity. The treatments are thus tolerant of overdosing within relatively wide limits. This property makes the treatments suitable for self-administration or other situations outside the clinical setting, a feature of neurological and other treatments which has hitherto not been available. The fact that the agent is a small molecule, and not a peptide (e.g. protein), further supports the potential utility of the present invention outside the clinical setting, where elaborate delivery apparatus for administration of peptide active agents directly into the brain or CNS may be unavailable.

Since many patients suffering from neurological, psychiatric, inflammatory, allergic, immune or neoplastic disorders may be relatively old, or in rather poor general health, they are often susceptible to some other disorders in these categories. Often it is not predictable with any certainty which of a range of other disorders or conditions will arise. Prior to the present invention, such other disorders or conditions, or the individual's susceptibility to them, contraindicated the treatment of the primary disorder, as very often the treatment would carry a substantial risk of promoting such other disorder or condition in such a patient. Therefore, the utility of the present invention in treating the disorders and conditions in ways that are easier and simpler than before, and which are applicable to a wider group of patients in this way, represents a substantial advance in medical science and healthcare practice in these important areas of human and animal health.

### BRIEF DESCRIPTION OF THE DRAWINGS

For further illustration, the data supporting the present invention will now be described, purely by way of example and without limitation.

In the accompanying drawings:

FIG. 1 shows the effect of smilagenin pre-treatment of neurones on the protection of the neurones against MPP⁺-induced damage;

FIG. 2 shows the effect of sarsasapogenin on (a) compound muscle action potential (CMAP) amplitude, (b) grid test and (c) survival in progressive motor neuropathy (pmn) mice; and

FIG. 3 shows the effect of sarsasapogenin, smilagenin and 4-methylcatechol on the CMAP (a) amplitude, (b) latency and (c) duration in nerve-damaged mice over time.

### EXAMPLES AND DETAILED DESCRIPTION OF THE DRAWINGS

In the following Examples and description of the drawings, the following abbreviations are used: h=hours; min=minutes; s=seconds, s.c.=subcutaneous, p.o=by mouth. Percentages for components of compositions which are solid in solid, or solid in liquid, or liquid in solid, are by weight. Percentages for components of compositions which are liquid in liquid are by volume.

### Example 1

### Sarsasapogenin and Smilagenin do not Bind to Several Enzymes and Receptors

The effect of sarsasapogenin on the activity of the enzymes listed in Table 1 below, and the binding of sarsasapogenin to the receptors listed in Table 1 below, were investigated.

The enzyme activity modulation was investigated using the following method: Sarsasapogenin was incubated with each enzyme plus a specific substrate for each enzyme. After the incubation period the reaction was stopped and the reduction of the specific substrate or the increase in a specific product in the absence and presence of sarsasapogenin was measured and the percent inhibition of the reaction in the presence of sarsasapogenin was calculated. The amount of enzyme used, the incubation conditions, the substrate used and the method of quantification varied depending on each specific assay.

The receptor binding was investigated using the following method: Sarsasapogenin was incubated with tissue or cell homogenate that expressed the receptor of interest and a known concentration a radiolabelled compound with affinity for the receptor of interest. After the incubation period the non-bound radiolabelled compound was removed and the amount of specific binding was quantified. The amount of specific binding in the presence and absence of sarsasapogenin were compared and the percent inhibition of the binding of the radiolabelled compound by sarsasapogenin was calculated. The source of the receptor, the incubation conditions, the radiolabelled compound used varied depending on each specific assay.

The results are shown in Table 1 below.

TABLE 1
Effect of sarsasapogenin on enzymes and receptor binding assays

| | | Sarsasapogenin | |
|---|---|---|---|
| Target | Species | (μM) | Effect (%) |
| Enzyme activity assays | | | |
| Acetylcholinesterase | Human | 10 | NS |
| Acetyl CoA synthetase | Yeast | 100 | NS |
| Choline acetyltransferase | Human | 100 | NS |
| Receptor binding assays | | | |
| Adrenergic α1, non selective | Rat | 10 | NS |
| Adrenergic α2, non selective | Rat | 10 | NS |
| Adrenergic β, non selective | Rat | 10 | NS |
| Dopamine D1 | Human | 10 | NS |
| Oestrogen | Bovine | 10 | NS |
| GABA_{A} | Rat | 10 | NS |
| Glucocorticoid | Human | 10 | NS |
| Glutamate | Rat | 10 | NS |
| Histamine H1 | Guinea Pig | 10 | NS |
| Muscarinic M1 | Human | 10 | NS |
| Muscarinic M2 | Human | 10 | NS |
| Muscarinic M3 | Human | 10 | NS |
| Muscarinic M4 | Human | 10 | NS |
| Muscarinic M5 | Human | 10 | NS |
| Progesterone | Bovine | 10 | NS |
| Serotonin 5-HT1 | Rat | 10 | NS |
| Testosterone | Rat | 10 | NS |

NS = No significant response.
Significance was taken as ≧30% stimulation or inhibition

Using the same methods as described above, the binding of smilagenin (1 μM) to the receptors listed in Table 2 below, and the effect of smilagenin on the activity of the enzymes listed in Table 2 below, were investigated.

TABLE 2
Effect of smilagenin on receptor binding assays and enzymes

| Target | Species | Effect (%) |
|---|---|---|
| Binding assays | | |
| Adensonine A₁ | Human | NS |
| Adensonine A_{2A} | Human | NS |
| Adensonine A₃ | Human | NS |
| Adrenergic α_{1A} | Rat | NS |
| Adrenergic α_{1B} | Rat | NS |
| Adrenergic α_{1D} | Human | NS |
| Adrenergic α_{2A} | Human | NS |
| Adrenergic α_{2C} | Human | NS |
| Adrenergic β₁ | Human | NS |
| Adrenergic β₂ | Human | NS |
| Adrenergic β₃ | Human | NS |
| Adrenomedullin AM₁ | Human | NS |
| Adrenomedullin AM₂ | Human | NS |
| Aldosterone | Rat | NS |
| Anaphylatoxin C5a | Human | NS |
| Androgen (testosterone) | Rat | NS |
| AR | | |
| Angiotensin AT₁ | Human | NS |
| Angiotensin AT₂ | Human | NS |
| APJ | Human | NS |
| Atrial Natriuretic Factor | Guinea pig | NS |
| Bombesin BB1 | Human | NS |
| Bombesin BB2 | Human | NS |
| Bombesin BB3 | Human | NS |
| Bradykinin B₁ | Human | NS |
| Bradykinin B₂ | Human | NS |
| Calcitonin | Human | NS |
| Calcitonin Gene-Related | Human | NS |
| Peptide CGRP₁ | | |
| Calcium Channel L-Type, | Rat | NS |
| Benzothiazepine | | |
| Calcium Channel L-Type, | Rat | NS |
| Dihydropyridine | | |
| Calcium Channel L-Type, | Rat | NS |
| Phenylalkylamine | | |
| Calcium Channel N-Type | Rat | NS |
| Cannabinoid CB₁ | Human | NS |
| Cannabinoid CB₂ | Human | NS |
| Chemokine CCR1 | Human | NS |
| Chemokine CCR2B | Human | NS |
| Chemokine CCR4 | Human | NS |
| Chemokine CCR5 | Human | NS |
| Chemokine CXCR1 | Human | NS |
| Chemokine CXCR1 (IL- | Human | NS |
| 8R_{B}) | | |
| Cholecystokinin CCK₁ | Human | NS |
| (CCK_{A}) | | |
| Cholecystokinin CCK₂ | Human | NS |
| (CCK_{B}) | | |
| Colchicine | | NS |
| Corticotropin Releasing | Human | NS |
| Factor (CRF₁) | | |
| Dopamine D₁ | Human | NS |
| Dopamine D_{2S} | Human | NS |
| Dopamine D₃ | Human | NS |
| Dopamine D_{4.2} | Human | NS |
| Dopamine D₅ | Human | NS |
| Endothelin ET_{A} | Human | NS |
| Endothelin ET_{B} | Human | NS |
| Epidermal Growth Factor | Human | NS |
| (EGF) | | |
| Erythropoietin EPOR | Human | NS |
| Oestrogen (ERα) | Human | NS |
| Oestrogen (Erβ) | Human | NS |
| G Protein-Coupled | Human | NS |
| Receptor GPR 103 | | |
| G Protein-Coupled | Human | NS |
| Receptor GPR8 | | |
| GABA_{A} Chloride | Rat | NS |
| Channel, TBOB | | |
| GABA_{A} Flunitrazepam, | Rat | NS |
| Central | | |
| GABA_{A} Muscimol, | Rat | NS |
| Central, | | |
| GABA_{B1A} | Human | NS |
| GABA_{B1B} | Human | NS |
| Gabapentin | Rat | NS |
| Galanin GAL1 | Human | NS |
| Galanin GAL2 | Human | NS |
| Glutamate, AMPA | Rat | NS |
| Glutamate, Kainate | Rat | NS |
| Glutamate, NMDA, | Rat | NS |
| Agonism | | |
| Glutamate, NMDA, | Rat | NS |
| Glycine | | |
| Glutamate, NMDA, | Rat | NS |
| Phencyclidine | | |
| Glutamate, NMDA, | Rat | NS |
| Polyamine | | |
| Glycine, Strychnine- | Rat | NS |
| Sensitive | | |
| Growth Hormone | Human | NS |
| Secretagogue (GHS, | | |
| Ghrelin) | | |
| Histamine H₁ | Human | NS |
| Histamine H₂ | Human | NS |
| Histamine H₃ | Human | NS |
| Histamine H₄ | Human | NS |
| Imidazoline I₂, central | Rat | NS |
| Inositol trisphosphate IP₃ | Rat | NS |
| Insulin | Rat | NS |
| Interleukin IL-1 | Mouse | NS |
| Interleukin IL-2 | Mouse | NS |
| Interleukin IL-6 | Human | NS |
| Leptin | Mouse | NS |
| Leukotriene, BLT (LTB₄) | Human | NS |
| Leukotriene, Cysteinyl | Human | NS |
| CysLT₁ | | |
| Leukotriene, Cysteinyl | Human | NS |
| CysLT₂ | | |
| Melanocortin MC₁ | Human | NS |
| Melanocortin MC₃ | Human | NS |
| Melanocortin MC₄ | Human | NS |
| Melanocortin MC₅ | Human | NS |
| Melatonin MT₁ | Human | NS |
| Melatonin MT₂ | Human | NS |
| Motilin | Human | NS |
| Muscarinic M₁ | Human | NS |
| Muscarinic M₂ | Human | NS |
| Muscarinic M₃ | Human | NS |
| Muscarinic M₄ | Human | NS |
| Muscarinic M₅ | Human | NS |
| N-Formyl Peptide | Human | NS |
| Receptor FPR1 | | |
| N-Formyl Peptide | Human | NS |
| Receptor-Like FPRL1 | | |
| Neuromedin U MNU₁ | Human | NS |
| Neuromedin U MNU₂ | Human | NS |
| Neuropeptide Y Y₁ | Human | NS |
| Neuropeptide Y Y₂ | Human | NS |
| Neurotensin NT₁ | Human | NS |
| Nicotinic Acetylcholine | Human | NS |
| Nicotinic Acetylcholine | Human | NS |
| α1, Bungarotoxin | | |
| Nicotinic Acetylcholine | Rat | NS |
| α7, Bungarotoxin | | |
| Opiate δ (OP1, DOP) | Human | NS |
| Opiate κ (OP2, KOP) | Human | NS |
| Opiate μ (OP3, MOP) | Human | NS |
| Orphanin ORL₁ | Human | NS |
| Phorbol Ester | Mouse | NS |
| Platelet Activating Factor | Human | NS |
| (PAF) | | |
| Platelet-Derived Growth | Mouse | NS |
| Factor (PDGF) | | |
| Potassium Channel [K_{A}] | Rat | NS |
| Potassium Channel | | NS |
| [K_{ATP}] | | |
| Potassium Channel | Rat | NS |
| [SK_{CA}] | | |
| Potassium Channel | Human | NS |
| HERG | | |
| Progesterone PR-B | Human | NS |
| Prostanoid CRTH2 | Human | NS |
| Prostanoid DP | Human | NS |
| Prostanoid EP₂ | Human | NS |
| Prostanoid EP₄ | Human | NS |
| Prostanoid Thromboxane | Human | NS |
| A₂ (TP) | | |
| Purinergic P_{2X} | | NS |
| Purinergic P_{2Y} | Rat | NS |
| Retanoid X Receptor | Human | NS |
| RXRα | | |
| Rolipram | Rat | NS |
| Ryanodine RyR3 | Rat | NS |
| 5-Hydroxytryptamine, 5- | Human | NS |
| HT_{1A} | | |
| 5-Hydroxytryptamine, 5- | Rat | NS |
| HT_{1B} | | |
| 5-Hydroxytryptamine, 5- | Human | NS |
| HT_{2B} | | |
| 5-Hydroxytryptamine, 5- | Human | NS |
| HT_{2C} | | |
| 5-Hydroxytryptamine, 5- | Human | NS |
| HT₃ | | |
| 5-Hydroxytryptamine, 5- | Guinea pig | NS |
| HT₄ | | |
| 5-Hydroxytryptamine, 5- | Human | NS |
| HT_{5A} | | |
| 5-Hydroxytryptamine, 5- | Human | NS |
| HT₆ | | |
| Sigma σ₁ | | NS |
| Sigma σ₂ | Rat | NS |
| Sodium Channel, Site 2 | Rat | NS |
| Somatostatin sstl | Human | NS |
| Somatostatin sst2 | Human | NS |
| Somatostatin sst3 | Human | NS |
| Somatostatin sst4 | Human | NS |
| Somatostatin sst5 | Human | NS |
| Tachykinin NK₁ | Human | NS |
| Tachykinin NK₂ | Human | NS |
| Tachykinin NK₃ | Human | NS |
| Thyroid Hormone | Rat | NS |
| Thyrotropin Releasing | Rat | NS |
| Hormone (TRH) | | |
| Transforming Growth | Mouse | NS |
| Factor-β (TGF-β) | | |
| Transporter, Adenosine | Guinea pig | NS |
| Transporter, Choline | Rat | NS |
| Transporter, Dopamine | Human | NS |
| (DAT) | | |
| Transporter, GABA | Rat | NS |
| Transporter, Monoamine | Rabbit | NS |
| Transporter, | Human | NS |
| Norepinephrine (NET) | | |
| Transporter, 5- | Human | NS |
| hydroxytryptamine | | |
| (SERT) | | |
| Tumour Necrosis Factor | Human | NS |
| (TNF), non-selective | | |
| Urotensin II | Human | NS |
| Vanilloid | Rat | NS |
| Vascular Endothelial | Human | NS |
| Growth Factor (VEGF) | | |
| Vasoactive Intestinal | Human | NS |
| Peptide, VIP₁ | | |
| Vasopressin V_{1A} | Human | NS |
| Vasopressin V_{1B} | Human | NS |
| Vasopressin V₂ | Human | NS |
| Vitamin D₃ | Human | NS |
| Functional assays | | NS |
| Protein Serine/Threonine | Human | NS |
| Kinase, AKT1 (PRKBA) | | |
| Protein Serine/Threonine | Human | NS |
| Kinase, AKT3 (PRKBG) | | |
| Protein Serine/Threonine | Human | NS |
| Kinase, CAMK2D | | |
| (KCC2D) | | |
| Protein Serine/Threonine | Human | NS |
| Kinase, MAP2K1 | | |
| (MEK1 ) | | |
| Protein Serine/Threonine | Human | NS |
| Kinase, MAPK1 (ERK2) | | |
| Protein Serine/Threonine | Human | NS |
| Kinase, MAPK11 (p38β) | | |
| Protein Serine/Threonine | Human | NS |
| Kinase, MAPK12 (p38γ) | | |
| Protein Serine/Threonine | Human | NS |
| Kinase, MAPK13 (p38δ) | | |
| Protein Serine/Threonine | Human | NS |
| Kinase, MAPK3 (ERK1) | | |
| Protein Serine/Threonine | Human | NS |
| Kinase, MAPK8 (JNK1) | | |
| Protein Serine/Threonine | Rat | NS |
| Kinase, PKC, Non- | | |
| Selective | | |
| Protein Tyrosine Kinase, | Human | NS |
| NTRK1 (trkA) | | |
| Protein Tyrosine Kinase, | Human | NS |
| NTRK2 (trkB) | | |
| Protein Tyrosine Kinase, | Human | NS |
| SRC | | |
| Aldose reductase | Rat | NS |
| Free Radical Scavenger, | abts-h\check{z}o\check{z}- | NS |
| ABTS Radical | peroxidase system | |
| Free Radical Scavenger, | chemical synthetic | NS |
| DPPH Radical | dpph radical | |
| Free Radical Scavenger, | Bovine | NS |
| SOD Mimetic | | |
| UDP | Human | NS |
| Glucuronosyltransferase, | | |
| UGT1A1 | | |

NS = No significant response.
Significance was taken as ≧30% stimulation or inhibition

Sarsasapogenin and smilagenin do not bind to a range of receptors and do not modulate the activity of a range of enzymes. Since these receptors and enzymes are known to be involved in neural, sensory and motor pathways, it is deduced that, within the limits of knowledge obtained from these experiments, the activity of sarsasapogenin and smilagenin against conditions and disorders having neural, sensory and motor origins does not arise through receptor binding or enzyme modulation.

### Example 2

### Sarsasapogenin and Smilagenin Transiently Increase Neurotrophic Factor mRNA in Cultured Neurones Under Basal Conditions In Vitro

Using specialised media and conditions, freshly isolated neurones can be cultured in vitro; the in vitro environment is different from the physiological one, resulting that the neurones are more stressed and suffer neuronal damage. The level of neuronal damage will vary from culture to culture depending on the precise conditions used. The level of neuronal damage can then be significantly increased by the addition of a pathological agent (e.g. β-amyloid or MPP⁺).

Rat cortical neurones were cultured by modification of a method previously described (Singer, et al., Neuroscience Letters, 1996, 212, pp. 13-16). Twelve days after the start of culturing, sarsasapogenin (30 nM), smilagenin (30 nM), 4-methylcatechol (0.5 mM), an inducer of NGF and BDNF release (Saporito et al., Experimental Neurology., 1993, 123, pp. 295-302; Nitta et al., Journal of Pharmacology and Experimental Therapeutics, 1999, 291, pp. 1276-1283) or vehicle (dimethyl sulfoxide, DMSO, 0.25%) were added for 1, 3 or 6 h. After incubation the total messenger ribonucleic acid (mRNA) was quantified using real time reverse transcription-polymerase chain reaction (rt RT-PCR).

The results are shown in Table 3 below.

TABLE 3
Effect of sarsasapogenin, smilagenin and 4-methylcatechol
on BDNF and trkB mRNA expression in rat cortical
neurones after 1, 3 and 6 h of incubation

| | | % increase above control | | |
|---|---|---|---|---|
| | Time | Sarsasapogenin | Smilagenin | 4-Methylcatechol |
| mRNA | (h) | (30 nM) | (30 nM) | (0.5 mM) |
| BDNF | 1 | No increase | No increase | No increase |
| | 3 | 22 | 40 | No increase |
| | 6 | No increase | No increase | 92 |
| trkB | 1 | No increase | 21 | No increase |
| | 3 | 33 | 55 | No increase |
| | 6 | No increase | No increase | No increase |

Both sarsasapogenin and smilagenin transiently (after 3 h) increase the level of mRNA of BDNF and the BDNF receptor trk-B (tyrosine receptor kinase neurotrophin receptor) in freshly isolated cortical neurones.

In a separate experiment, rat cortical neurones were cultured by modification of a method previously described (Eckenstein and Sofroniew, Journal of Neuroscience, 1983, 3, pp. 2286-2291). On day 8, the culture medium was changed to a medium containing vehicle (DMSO, 0.5%) or smilagenin (10 μM) for 48 h and the level of BDNF mRNA in the cortical neurones was assessed by rt RT-PCR.

The results are shown in Table 4 below.

TABLE 4
Effect of 48 h incubation with sarsasapogenin on
BDNF mRNA expression in rat cortical neurones.

| | Relative amount of BDNF mRNA |
|---|---|
| Condition | (% of control) |
| Control (DMSO, 0.5%) | 100.0 ± 0.0 |
| Smilagenin (10 μM) | 103.9 ± 6.5 |

Mean ± s.e.mean; n = 3.

Incubation for 48 h with smilagenin (10 μM) did not increase the level of BDNF mRNA in freshly isolated cortical neurones. This is in agreement with the data presented in Table 3 that showed that smilagenin and sarsasapogenin increased BDNF mRNA at 3 h but not at 6 h. In addition, the transient effect of smilagenin (Table 3) was not overcome by a high concentration of smilagenin (Table 4).

### Example 3

### Smilagenin Causes a Significant Increase in Neurotrophic Factor mRNA Expression in Cultured Neurones Exposed to a Pathological Agent In Vitro

Smilagenin Increases BDNF mRNA in Cortical Neurones Previously Exposed to β-Amyloid

Rat cortical neurones were cultured by modification of a method previously described (Eckenstein and Sofroniew, Journal of Neuroscience, 1983, 3, pp. 2286-2291). On day 8, the culture medium was changed to a medium containing vehicle (DMSO, 0.5%) or smilagenin (10 μM). On day 10, rat primary cortical neurones were exposed to β-amyloid (10 μg/ml) for up to 48 h at 37° C. and the level of BDNF mRNA in the cortical neurones was assessed by rt RT-PCR over the following 48 h.

The results are shown in Table 5 below.

TABLE 5
Pre-incubation with smilagenin for 48 h followed by exposure
to β-amyloid increases BDNF mRNA in rat cortical neurones

| | Relative amount of BDNF mRNA | |
|---|---|---|
| Length of | (% of control at 0 h) | |
| β-amyloid | Vehicle + β-amyloid | Smilagenin (10 μM) + β-amyloid |
| exposure (h) | (10 μg/ml) | (10 μg/ml) |
| 6 | 97.6 ± 1.3 | 108.0 ± 5.2 |
| 24 | 77.7 ± 3.6^{##} | 321.5 ± 54.2* |
| 48 | 60.9 ± 5.0^{##} | 334.6 ± 48.1** |

Mean ± s.e.mean; n = 3,
**= p < 0.01,
*= p < 0.05 compared to the corresponding time point of β-amyloid alone. Statistical analysis was by a Student's t-test.

Pretreatment with smilagenin for 48 h followed by β-amyloid exposure produced a significant and sustained increase in the expression of BDNF mRNA in rat cortical neurones.

Smilagenin Increases GDNF mRNA in Dopaminergic Neurones Previously Exposed to MPP⁺

Rat dopaminergic neurones were prepared using a slightly modified previously described method (Brouard et al., Journal of Neuroscience, 1992, 12, pp. 1409-1415). After 5 days in culture MPP⁺ (2 μM), a specific dopaminergic neurotoxin, or vehicle (saline) was added for 48 h. The culture medium was then replaced with fresh medium containing smilagenin (10 μM) or vehicle (DMSO, 0.25%) and the level of GDNF mRNA in the dopaminergic neurones was assessed after 10 min and 2, 24, 48 and 72 h by rt RT-PCR.

The results are shown in Table 6 below.

TABLE 6
Smilagenin increases GDNF mRNA expression in rat
dopaminergic neurones following exposure to MPP⁺.

| | |
|---|---|
| | Relative amount of GDNF mRNA |
| Length of | (% of control at 0.167 h) |

| smilagenin | | MPP⁺ + smilagenin |
|---|---|---|
| exposure (h) | MPP⁺ (2 μM) | (10 μM) |
| 0.167 | 100.0 ± 0.0 | 119.9 ± 19.8 |
| 2 | 90.1 ± 13.4 | 581.6 ± 66.3** |
| 24 | 107.0 ± 25.0 | 3319.1 ± 830.3* |
| 48 | 97.8 ± 33.5 | 2185.3 ± 304.1** |
| 72 | 77.2 ± 15.6 | 1413.5 ± 352.1* |

Mean ± s.e.mean; n = 3
**= p < 0.01,
*= p < 0.05 compared to control. Statistical analysis of the GDNF mRNA between control and smilagenin at each time point was by a Student's t-test.

Treatment with smilagenin for 48 h after exposure to MPP⁺ caused a significant increase of GDNF mRNA expression in rat dopaminergic neurones. The increase was maximal at 24 h and then declined after 48 and 72 h.

Examples 2 and 3 demonstrate that smilagenin and sarsasapogenin increase neurotrophic factor mRNA expression. Furthermore, the effect of smilagenin and sarsasapogenin on neurotrophic factors mRNA expression varies in its extent (duration and magnitude) depending on the condition of the neurones. In cultured neurones under basal conditions smilagenin and sarsasapogenin produced a transient increase (up to 140% of control) in neurotrophic factor RNA levels which was observed at 3 h (Table 3) but not at 6 h (Table 3) or 48 h (Table 4). By contrast, in cultured neurones exposed to a pathological agent (e.g. (β-amyloid or MPP⁺) smilagenin produced a more pronounced (up to 3319% of control) and for a longer duration (for up to 72 h) increase of neurotrophic factor mRNA expression and (Tables 5 and 6). The results demonstrate that the neurotrophic inducer effects of sarsasapogenin and smilagenin self-regulate themselves depending on the degree of damage to the system. i.e. that sarsasapogenin and smilagenin do not disrupt or override the self-regulatory mechanism of neurotrophic factors.

### Example 4

### Smilagenin does not Alter Neurotrophic Factor Protein Expression in Cultured Neurones Under Basal Conditions In Vitro

Rat cortical neurones were cultured by modification of a method previously described (Eckenstein and Sofroniew, Journal of Neuroscience, 1983, 3, pp. 2286-2291). On day 8, the culture medium was changed to a medium containing vehicle (DMSO, 0.5%) or smilagenin (10 μM). On day 12 the concentration of BDNF in the culture medium, was measured.

The results are shown in Table 7 below.

TABLE 7
Incubation with smilagenin does not alter BDNF protein level
in cultured neurones under basal conditions in vitro

| Condition | BDNF concentration (pg/ml) |
|---|---|
| Control (DMSO, 0.5%) | 3.66 ± 0.05 |
| Control + smilagenin (10 μM) | 3.67 ± 0.05 |

Mean ± s.e.mean; n = 6

Smilagenin does not increase BDNF levels in cultured neurones under basal conditions in vitro.

### Example 5

### Sarsasapogenin and Smilagenin Increase Neurotrophic Factor Protein Expression in Cultured Neurones Exposed to a Pathological Agent In Vitro

### Sarsasapogenin and Smilagenin Increase BDNF Protein and Increase Neuronal Survival and Neurite Outgrowth in Cortical Neurones Previously Exposed to β-Amyloid

Rat cortical neurones were cultured by modification of a method previously described (Eckenstein and Sofroniew, Journal of Neuroscience, 1983, 3, pp. 2286-2291). On day 8, the culture medium was changed to a medium containing vehicle (DMSO, 0.5%) or smilagenin or sarsasapogenin (10 μM). On day 10, rat primary cortical neurones were exposed to β-amyloid (10 μg/ml) for 48 h at 37° C. and the concentration of BDNF in the culture medium, the number of choline acetyltransferase (ChAT) positive cells, and the neurite outgrowth was measured (smilagenin only).

The results are shown in Table 8 below.

TABLE 8
Pre-incubation with smilagenin or sarsasapogenin for 48 h followed by β-amyloid exposure
increases the BDNF protein level and prevents neuronal damage and neuronal atrophy in vitro.

| | | Number of ChAT | Neurite |
|---|---|---|---|
| | BDNF concentration | positive neurones per | outgrowth |
| Condition | (pg/ml) | field (% of control) | (% of control) |
| Sarsasapogenin results | | | |
| Control (DMSO, 0.5%) | 7.35 ± 0.18 | 100.0 ± 3.6 | n.m. |
| β-amyloid (10 μg/ml) | 1.94 ± 0.06⁺⁺⁺⁺ | 33.9 ± 1.4⁺⁺⁺⁺ | n.m. |
| β-amyloid + | 9.31 ± 0.15^{++++,}**** | 71.6 ± 3.7**** | n.m |
| sarsasapogenin (10 μM) | | | |
| Smilagenin results | | | |
| Control (DMSO, 0.5%) | 3.66 ± 0.05 | 100.0 ± 11.9 | 100.0 ± 1.7 |
| β-amyloid (10 μg/ml) | 3.10 ± 0.05⁺⁺⁺⁺ | 29.9 ± 4.4⁺⁺⁺⁺ | 39.5 ± 2.2⁺⁺⁺⁺ |
| β-amyloid + smilagenin (10 μM) | 4.20 ± 0.06****^{,++++} | 70.1 ± 9.6*** | 85.8 ± 4.0**** |

n.m. = not measured; Mean ± s.e.mean; n = 4-8,
⁺⁺⁺⁺= p < 0.001 compared to control,
****= p < 0.001,
***= p < 0.005 compared to β-amyloid alone. Statistical analysis was performed using one-way ANOVA, followed by Fisher's post-hoc test.

Sarsasapogenin and smilagenin increase BDNF level above control levels and prevent β-amyloid-induced neuronal damage in cortical neurones.

### Smilagenin Increases GDNF Protein and Increases Neuronal Survival and Neurite Outgrowth in Dopaminergic Neurones Previously Exposed to MPP⁺

Rat dopaminergic neurones were prepared using a slightly modified previously described method (Brouard et al., Journal of Neuroscience, 1992, 12, pp. 1409-1415). On day 6 the culture medium was replaced with fresh medium or fresh medium containing smilagenin (10 μM) or vehicle (DMSO, 0.25%). On day 8, MPP⁺ (2 μM) or vehicle (saline) was added and 48 h later dopaminergic neurones were stained and the concentration of GDNF in the culture medium, neuronal damage and neurite outgrowth were assessed.

The results are shown in Table 9 below.

TABLE 9
Smilagenin increases the amount of GDNF in the culture
medium and prevents neuronal damage and neuronal atrophy following
MPP⁺ exposure in rat dopaminergic neurones.

| | | Number of TH | Neurite |
|---|---|---|---|
| | GDNF | positive neurones | outgrowth |
| | concentration | per field (% | (% of |
| Condition | (pg/ml) | of control) | control) |
| Control | n.m. | 100 ± 9.9 | 100 ± 10.1 |
| (DMSO, 0.25%) | | | |
| MPP⁺ (2 μM) | 2.7 ± 0.5 | 34.3 ± 3.3⁺⁺⁺⁺ | 38.2 ± 4.2⁺⁺⁺⁺ |
| MPP⁺ (2 μM) + | 6.6 ± 0.7** | 57.1 ± 5.2* | 62.1 ± 7.0* |
| Smilagenin (10 μM) | | | |

n.m. = not measured; mean ± s.e.mean; n = 5-6,
⁺⁺⁺⁺= p < 0.001 compared to control,
*= p < 0.05 compared to MPP⁺ alone. Statistical analysis of the number of TH positive neurones and neurite outgrowth was performed using one-way ANOVA, followed by Fisher's post-hoc test. Statistical analysis of the GDNF concentration was by a Student's t-test.

Smilagenin increases the amount of GDNF and prevents MPP⁺-induced neuronal damage in dopaminergic neurones.

The data presented in Example 4 shows that smilagenin does not increase neurotrophic factor protein expression in cultured neurones under basal conditions in vitro. By contrast, Example 5 shows that both sarsasapogenin and smilagenin increase neurotrophic factor protein expression in cultured neurones exposed to a pathological agent in vitro. Therefore, the effect of sarsasapogenin and smilagenin on neurotrophic factor protein is similar to their effect on neurotrophic factor mRNA, i.e. that sarsasapogenin and smilagenin do not disrupt or override the self-regulatory mechanism of neurotrophic factors but are in fact subject to them depending on the requirements of the neurones.

Since BDNF, trk-B and GDNF are known to be involved in neural, sensory and motor pathways, it is deduced that, within the limits of knowledge obtained from these experiments, the activity of sarsasapogenin and smilagenin against conditions and disorders having neural, sensory and motor origins involves enhanced gene expression of neurotrophic factors and their receptors.

### Example 6

### Sarsasapogenin and Smilagenin Restore BDNF Concentration in Aged Animals

Old Sprague Dawley (SD) rats (20 month old) were orally administered sarsasapogenin or smilagenin (18 mg/kg/day) for 3 months. BDNF is significantly reduced in aged rat brain compared to young rat brain. Young SD rats (4 month old) were used as healthy positive control. At the end of the treatment the brains removed for quantification of BDNF using an ELISA.

The results are shown in Table 11 below.

TABLE 11
Sarsasapogenin and smilagenin reverse the decline of BDNF levels
in aged rats and restore BDNF levels towards the young state

| Condition | BDNF (ng/g tissue) |
|---|---|
| Young | 1.65 ± 0.09 |
| Aged | 1.21 ± 0.01 ⁺⁺⁺⁺ |
| Aged + sarsasapogenin (18 mg/kg/day) | 1.41 ± 0.07 * |
| Aged + smilagenin (18 mg/kg/day) | 1.34 ± 0.07 * |

Mean ± s.e.mean; n = 9-10, statistical analysis performed using paired one-tailed Student's t-test
⁺⁺⁺⁺ = p < 0.001 compared to young rats.
* = p < 0.05. compared to aged rats.

Sarsasapogenin or smilagenin, orally administered to aged rats for 3 months, reverse the decline in BDNF of aged animals towards the levels observed in young healthy rats, i.e. the agents significantly increase BDNF levels compared to aged control rats.

This data indicates that the effect of the agents on BDNF expression is a normalising effect under long term administration, i.e. that there is a long term regulatory effect protecting the treated animal against overexposure to the agent, by limiting the recovery to approximately the normal state.

This Example complements the experiment in Example 9 of PCT Patent Application No. WO-A-03/082893, incorporated herein by reference. That experiment demonstrated that age-related BDNF, dopamine receptor and muscarinic acetylcholine receptor decline in rats was significantly reduced or reversed with smilagenin or sarsasapogenin.

### Example 7

### Smilagenin Increases BDNF and GDNF Concentration in the Striatum of MPTP-Lesioned Mice

Seven-week old male C57b1/6 RJ mice (C57 mice) received daily injections 1-methyl-4-phenyl-1,2,3,6-tetrahydropyridine (MPTP, 25 mg/kg/day, i.p., for 5 consecutive days) and oral administration of smilagenin (10 mg/kg/day) or vehicle (hydroxylpropylmethylcellulose, HPMC 0.5% w/v containing tween-80 0.2% v/v) for 60 days after which time their brains were removed for quantification of striatal levels of GDNF and BDNF using an ELISA and of dopamine transporter (DAT) levels using [I^{125]}-RTI binding. DAT is a marker for neuronal damage to dopaminergic neurones.

Damage caused by the neurotoxin MPP⁺, a metabolite of MPTP, mimics the degeneration of nigrostriatal dopaminergic neurones observed in neurodegenerative diseases such as Parkinson's disease (Mytinlineou et al, Science, 225, 529-531 (1984)). The most prominent biochemical changes induced by this toxin include increased levels of dopamine and its metabolites in the substantia nigra pars compacta and in the caudate nucleus (Burns et al, Proc. Natl. Acad. Sci. USA, 80, 4546-4550 (1983)) and a reduction in dopamine uptake in nigrostriatal synaptosomal preparations (Heikkila et al, J. Neurochem., 44, 310-313 (1985)).

The MPTP treated mice used in this experiment thus provide an accepted model for Parkinson's disease and similar motor-sensory neurodegenerative conditions.

The results are shown in Tables 12 and 13 below.

TABLE 12
Smilagenin increases striatal GDNF
and BDNF in MPTP-lesioned mice

| | | GDNF (% increase |
|---|---|---|
| | GDNF (pg/mg tissue) | above MPTP mice) |
| MPTP mice | 58.36 ± 15.32 | — |
| MPTP + smilagenin | 275.31 ± 59.62** | 372 ± 92** |
| | | BDNF (% increase |
| | BDNF (pg/mg tissue) | above MPTP mice) |
| MPTP mice | 17.75 ± 4.80 | — |
| MPTP mice + smilagenin | 46.91 ± 9.97** | 164 ± 53** |

Mean ± s.e.mean; n = 8-11
**= p < 0.01 compared to MPTP-lesioned mice. Statistical analysis performed using one-way ANOVA, followed by Tukey's post-hoc multiple comparison test.

TABLE 13
Smilagenin increases striatal DAT levels in MPTP-lesioned mice

| | DAT level ([I¹²⁵]-RTI |
|---|---|
| | binding in the striatum; |
| Condition | nCi/g protein) |
| Control mice | 74.4 ± 4.9 |
| MPTP mice | 23.4 ± 3.9⁺⁺ |
| MPTP + smilagenin (10 mg/kg/day) | 69.7 ± 8.8** |

Mean ± s.e.mean; n = 6-8
⁺⁺= p < 0.01 compared to control mice;
**= p < 0.01 compared to MPTP-lesioned mice. Statistical analysis performed using one-way ANOVA, followed by Tukey's post-hoc multiple comparison test.

Orally administered smilagenin to MPTP-lesioned mice for 60 days significantly elevate striatal GDNF and BDNF levels and significantly prevents MPTP-induced loss of DAT binding.

This Example complements the in vitro experiments in Examples 6 and 7 of PCT Patent Application No. WO-A-03/082893, incorporated herein by reference. Those experiments demonstrated that pre-treatment of rat mesencephalic dopaminergic neurones with smilagenin or sarsasapogenin significantly prevented or reversed MPP⁺-induced neurodegeneration in vitro.

In a similar experiment, 10-week old male C57 mice received daily injections of saline or MPTP (25 mg/kg/day, i.p.) for 5 consecutive days (days 1-5) and oral administration of smilagenin (10 mg/kg/day) or vehicle (HPMC 0.5% w/v containing tween-80 0.2% v/v) for 61 days (days 12-72) or 71 days (days 2-72) after which time their brains were removed for quantification of striatal levels of DAT, marker of the extent of neuronal damage to dopaminergic neurones

The results are shown in Table 14 below.

TABLE 14
Smilagenin reverses MPTP-induced reductions
in striatal DAT levels in mice

| | DAT level ([I¹²⁵]-RTI binding in the |
|---|---|
| | striatum; nCi/g protein) |
| Control | 435.9 ± 20.4 |
| Control + smilagenin | 406.2 ± 21.8 |
| (10 mg/kg/day, days 2-72) | |
| MPTP | 158.2 ± 24.9**** |
| MPTP + smilagenin | 280.3 ± 18.3⁺⁺⁺⁺ |
| (10 mg/kg/day, days 12-72) | |
| MPTP + smilagenin | 256.2 ± 21.4⁺⁺⁺⁺ |
| (10 mg/kg/day, days 2-72) | |

Mean ± s.e.mean; n = 8-12,
****= p < 0.001, compared to control mice,
⁺⁺⁺⁺= p < 0.001 compared to MPTP mice. Statistical analysis performed using one-way ANOVA, followed by Fisher's post-hoc multiple comparison test.

Orally administered smilagenin to control mice for 71 days does not alter striatal DAT level compared to control mice receiving vehicle alone. Orally administered smilagenin to MPTP-lesioned mice for 61 or 71 days significantly reverses MPTP-induced reductions in striatal DAT levels.

### Example 8

### Sarsasapogenin and Smilagenin Increase Neuritogenesis in Cortical, Spinal Motor and Sensory Neurones

### Cortical Neurones

Rat cortical neurones were cultured by modification of a previously described method (Singer, et al., Neuroscience Letters, 1996, 212, pp. 13-16). Cells were cultured with sarsasapogenin, smilagenin, vehicle (DMSO 0.25%), GDNF, BDNF or NGF for 24 h. For each group, 15 pictures showing neurones displaying neurites were selected at random in each field, and for each neurone the longest neurite was measured. The neurite number was measured by counting the number of neurones displaying neurites, the number of neurones not displaying neurites and the number of total neurones in each field. Six fields per well were examined.

The results are shown in Table 15 below, expressed as the number of neurones with neurites per field as a percentage of the total number of neurones per field.

TABLE 15
Sarsasapogenin and smilagenin increase
neuritogenesis in cortical neurones
Cortical neurones

| | Neurite length | Neurones displaying |
|---|---|---|
| Condition | (% of control) | neurites (%) |
| Control | 100.00 ± 4.76 | 39.35 ± 2.06 |
| Sarsasapogenin (3 nM) | 168.29 ± 6.12**** | 52.40 ± 2.63**** |
| Sarsasapogenin (30 nM) | 156.74 ± 4.06**** | 57.32 ± 2.54**** |
| Smilagenin (3 nM) | 159.03 ± 4.91**** | 53.84 ± 2.93**** |
| Smilagenin (30 nM) | 176.71 ± 6.34**** | 53.46 ± 2.13**** |
| GDNF (3 nM) | 125.30 ± 4.18*** | 55.72 ± 1.98**** |
| BDNF (3 nM) | 162.34 ± 5.91**** | 48.06 ± 2.17** |
| NGF (3 nM) | 145.73 ± 5.13**** | 52.59 ± 2.43**** |

Mean ± s.e.mean; one culture, n = 3, statistical analysis performed using one-way ANOVA followed by Fisher's post-hoc test.
**= p < 0.01;
***= p < 0.005,
****= p < 0.001, compared to control

Sarsasapogenin and smilagenin significantly increase the length of existing neurites and the percentage of neurones displaying neurites in rat primary cortical neurones. The effect in increasing neurite outgrowth following exposure to sarsasapogenin and smilagenin is comparable to that observed with the positive controls, GDNF, BDNF and NGF.

This Example complements the experiment in Example 5 of PCT Patent Application No. WO-A-03/082893, incorporated herein by reference. That experiment demonstrated that treatment of rat primary cortical neurones with sarsasapogenin or smilagenin significantly increased the length of existing neurites and the percentage of neurones displaying neurites.

To test whether the neurotrophic, neuroprotective and neurorestorative activities of sarsasapogenin and smilagenin are dependent upon the presence of neurotrophic factors such as BDNF or GDNF, the following experiment was performed:

Rat cortical neurones were cultured following the method described above.

Differently from previous studies, there was no foetal bovine serum (FBS) or foetal calf serum (FCS) added in the culture medium, indicating that no neurotrophic factors were present in the culture. The test compounds were added for 24 h.

Rat cortical neurones were exposed to sarsasapogenin, smilagenin (30 nM) or vehicle (DMSO, 0.25%) in the absence FBS or FCS for one day. Cortical neurones were stained using a monoclonal antibody anti β-tubulin diluted and an anti mouse Immunoglobulin G diluted. These antibodies stained neurone cell bodies (quantifying the neuroprotective effect) and neurites (quantifying the neurotrophic effect). An epifluorecence microscope (magnification ×20) with a camera took 2 pictures per well (10 pictures per condition). Analyses of the number of cells labelled with anti β-tubulin antibodies and of the total number of cells was performed using LUCIA 6.0 software.

The results are shown in Table 16 below.

TABLE 16
Effect of sarsasapogenin and smilagenin on neuronal survival and
neurite outgrowth of cortical neurones cultured in the absence
of serum and any additional neurotrophic factors
Cortical neurones

| | Neuronal survival | Neurite outgrowth |
|---|---|---|
| Condition | (% of control) | (% of control) |
| Control | 100.00 ± 4.05 | 100.00 ± 3.82 |
| Sarsasapogenin (30 nM) | 149.55 ± 6.22**** | 152.85 ± 10.68**** |
| Smilagenin (30 nM) | 155.36 ± 4.75**** | 173.89 ± 9.23**** |
| BDNF (3 nM) | 162.75 ± 5.61**** | 146.84 ± 9.27**** |

Mean ± s.e.mean; n = 12 wells per culture, n = 2 cultures were used. Statistical analysis was performed by one-way ANOVA, followed by Fisher's post-hoc test,
****= p < 0.001 compared to control

Sarsasapogenin and smilagenin do not need additional neurotrophic factors to exert their neurotrophic, neuroprotective and neurorestorative activities.

### Spinal Motor Neurones

Xaliproden (1-[2-(naphth-2-yl)ethyl]-4-(3-trifluoromethylphenyl)-1,2,5,6-tetra-hydropyri-dine hydrochloride), also known as SR 57746A, is an orally active, synthetic, non-peptide compound developed by Sanofi-Aventis for the treatment of neurodegenerative diseases. Xaliproden penetrates the blood-brain barrier and has neurotrophic activity in vitro, where it potentiates the effect of NGF on neurite outgrowth in PC12 cells (Fournier et al., Neuroscience, 1993, 55, pp. 629-641; Pradines et al., Journal of Neurochemistry, 1995, 64, pp. 1954-1964) and increases the survival of mouse spinal motor neurones (Duong et al., British Journal of Pharmacology, 1999, 128, pp. 1385-1392). Furthermore, Xaliproden increases the mean survival time and the motor performance of progressive motor neuropathy mice (Duong et al., British Journal of Pharmacology, 1998, 124, pp. 811-817). The mode of action of Xaliproden is poorly understood. However, the neuroprotective effect of Xaliproden appears independent of its agonist action at the 5-hydroxytryptamine_{1A} receptor (Labie et al., British Journal of Pharmacology, 1999, 127, pp. 139-144).

The following experiment compares the neurogenic and neuritogenic effect of sarsasapogenin or smilagenin against Xaliproden

Rat spinal motor neurones were prepared according to a previously described method (Martinou et al, Neuron, 8, 737-744, 1992). Following 3 days of culturing with sarsasapogenin, smilagenin, vehicle (DMSO, 0.25%), Xaliproden, or BDNF, spinal cord motor neurones were washed twice in PBS, fixed in a cold solution of alcohol (95%) and acetic acid (5%) for 5 min and then rinsed 3 times in PBS. Neurones were stained using a monoclonal antibody anti β-tubulin and an anti mouse Immunoglobulin G. These antibodies stained neurone cell bodies (quantifying the neuroprotective effect) and neurites (quantifying the neurotrophic effect). The cell nuclei were stained by a fluorescent marker. After 1 h of incubation, cells were washed 3 times in PBS. Cultures were observed with an epifluorescence microscope with 20-fold magnification. A series of pictures were taken using a camera controlled by computer software. All the images were taken under the same conditions. Analyses of the number of cells labelled with anti β-tubulin antibodies and of the total number of cells (number of stained nuclei) were performed using LUCIA 6.0 software.

The results are shown in Table 17 below.

TABLE 17
Sarsasapogenin and smilagenin increase neurogenesis
and neuritogenesis in spinal motor neurones
Spinal motor neurones

| | Neuronal survival | Neurite outgrowth |
|---|---|---|
| Condition | (% of control) | (% of control) |
| Control | 100.00 ± 2.16 | 100.00 ± 7.82 |
| Sarsasapogenin (30 nM) | 119.14 ± 3.33**** | 129.98 ± 5.47** |
| Sarsasapogenin (100 nM) | 117.87 ± 3.63**** | 137.38 ± 7.93*** |
| Smilagenin (30 nM) | 120.11 ± 2.92**** | 163.66 ± 9.28**** |
| Smilagenin (100 nM) | 121.21 ± 2.75**** | 164.75 ± 5.57**** |
| Xaliproden (30 nM) | 110.95 ± 2.14** | 137.57 ± 11.69*** |
| Xaliproden (100 nM) | 111.18 ± 2.85** | 137.55 ± 6.76*** |
| Xaliproden (300 nM) | 109.47 ± 3.34* | 131.22 ± 7.93** |
| BDNF (1.85 nM) | 126.15 ± 1.60**** | 176.91 ± 7.25**** |

Mean ± s.e.mean; n = 12 wells per culture, n = 2 cultures were used. Statistical analysis was performed by one-way ANOVA, followed by Fisher's post-hoc test,
*= p < 0.05,
**= p < 0.01,
***= p < 0.005 and
****= p < 0.001 compared to control.

The data show that exposure to Xaliproden significantly increased neuronal survival and neurite outgrowth compared to control. Sarsasapogenin and smilagenin also significantly increased neuronal survival and neurite outgrowth in the rat primary spinal motor neurones. The effect in increasing neuritogenesis is comparable to that observed with the positive control BDNF.

The effect of sarsasapogenin and smilagenin to promote neurogenesis appears slightly more pronounced than the effect of Xaliproden; although, the effect of sarsasapogenin and smilagenin appears reduced in this study compared to previous studies.

Efficacy and safety of Xaliproden (1 and 2 mg/day) has been assessed in two phase III clinical trials using amyotrophic lateral sclerosis (ALS) patients (Meininger et al., Amyotrophic Lateral Sclerosis and Other Motor Neuron Disorders, 2004, 5, pp. 107-117). In addition, Xaliproden was recently evaluated in a Phase III trial as a potential for Alzheimer's disease, an indication for which Xaliproden is now no longer being progressed. Dose-dependent side effects were largely associated with the 5-hydroxytryptamine (5-HT) agonist properties of Xaliproden.

In the present Example, sarsasapogenin and smilagenin showed an improved or similar activity profile compared to Xaliproden. Importantly, sarsasapogenin and smilagenin are not 5-HT agonists, and do not show the corresponding side effects of Xaliproden.

This Example complements the in vitro experiment in Example 8 of PCT Patent Application No. WO-A-03/082893, incorporated herein by reference. That experiment demonstrated that glutamate-induced neurodegeneration of rat primary spinal motor neurones in vitro was significantly reduced or reversed with sarsasapogenin or smilagenin.

### Sensory Neurones

Rat sensory neurones were obtained from Wistar rat embryos on the 15^{th} day of gestation. Cells were cultured at 37° C. in 5% CO₂/95% air atmosphere. Following 2 days of culturing with sarsasapogenin, smilagenin, vehicle (DMSO, 0.25%) or NGF, sensory neurones were rinsed twice with PBS and fixed in paraformaldehyde (4%) in PBS for 30 min at 4° C. Cells were permeabilised with Triton X-100 (0.1%) and non-specific sites were saturated using foetal bovine serum. Prior to staining, cells were incubated for 2 h at room temperature with a mixture of primary antibodies: anti-neurofilament 68 and 200 in PBS containing foetal bovine serum 5%. Prior to washing, slides were demounted and the cells were washed twice with PBS for 5 min, placed in a dark room for 1 h and incubated with a secondary antibody: anti-mouse coupled with cyanine 3 (Cy3; 1/1600) and an anti-rabbit coupled with fluoroisothiocyanate (FITC; 1/200) in PBS containing foetal bovine serum (5%). Slides were washed twice with PBS for 5 min and mounted on coverslips using Mowiol, an antioxidative solution (9% w/v) in glycerol (22%) buffered with Tris/HCl (0.2 mM; pH 8.5). Slides were left overnight at room temperature to harden and stored in light protected conditions. Slides were viewed using a DAPI/FITC/Cy3 triple filter microscope with a ×20 objective. A series of photographs per well were taken at random using a digital camera.

The results are shown in Table 18 below.

TABLE 18
Sarsasapogenin and smilagenin increase
neuronal survival in sensory neurones
Sensory neurones

| | Neuronal survival |
|---|---|
| Conditions | (% of control) |
| Control | 100.00 ± 5.34 |
| Sarsasapogenin (30 nM) | 130.83 ± 1.75**** |
| Smilagenin (300 nM) | 124.02 ± 7.64* |
| NGF (0.2 nM) | 151.50 ± 7.19**** |

Mean ± s.e.mean; n = 40-49 wells, n = 2 cultures were used. Statistical analysis was performed by one-way ANOVA, followed by Fisher's post-hoc test,
*= p <0.05 and
****= p < 0.001 compared to control

Sarsasapogenin and smilagenin significantly increases neuronal survival in rat primary sensory neurones.

### Example 9

### Sarsasapogenin and Smilagenin Activate the Same Intracellular Transduction Pathways As Neurotrophic Factors

The sarsasapogenin and smilagenin-induced neuritogenesis is inhibited by K252a, a trk inhibitor, suggesting that the neurotrophic effects of sarsasapogenin and smilagenin are directly or indirectly mediated via trk receptors. This inhibition experiment is described below and the results are shown in Table 19 below.

Cortical neurones were cultured as detailed above. Neurones were exposed to vehicle (DMSO, 0.25%) or K252a (100 nM) for 1 h. After 1 h, vehicle, sarsasapogenin, smilagenin (30 nM) or BDNF (1.85 nM) was added to the medium in the maintained presence of K252a. Following 24 h exposure to sarsasapogenin or smilagenin (30 nM), vehicle (DMSO, 0.25%) or BDNF (1.85 nM), the neurones were washed using phosphate-buffered saline (PBS) and fixed in glutaraldehyde (2.5%) in PBS. Photographs of 40-60 neurones expressing neurites were taken with a camera fixed on a microscope (objective ×20, Nikon). The neurite length was measured by an analysis of the photographs.

The results are shown in Table 19 below.

TABLE 19
Inhibition of sarsasapogenin and smilagenin-induced
neurite outgrowth of rat primary cortical neurones
Cortical neurones

| | Neurite length |
|---|---|
| Condition | (% of control) |
| Control | 100.00 ± 3.18 |
| Control with K252a (100 nM) | 95.22 ± 3.10 |
| Sarsasapogenin (30 nM) | 126.74 ± 5.76⁺⁺⁺⁺ |
| Sarsasapogenin (30 nM) with K252a (100 nM) | 93.12 ± 2.88**** |
| Smilagenin (30 nM) | 146.78 ± 6.75⁺⁺⁺⁺ |
| Smilagenin (30 nM) with K252a (100 nM) | 94.36 ± 3.96**** |
| BDNF (1.85 nM) | 125.30 ± 5.80⁺⁺⁺⁺ |
| BDNF (1.85 nM) with K252a (100 nM) | 87.31 ± 2.20**** |

Mean ± s.e.mean; n = 81-105 neurones per culture, n = 2 were used, statistical analysis performed using one-way ANOVA followed by Fisher's post-hoc test.
⁺⁺⁺⁺= p < 0.001 compared to control;
****= p < 0.001 compared to the same condition without K252a

Similar results were obtained in independent experiments using K252a, anti-BDNF or anti-GDNF antibodies in cortical and mesencephalic neurones.

Following trk receptor activation, specific signal transduction pathways are activated that lead to neuronal survival and MEK1/2 has been shown to be involved in this pathway (Finkbeiner, Neuron, 2000, 25, pp. 11-14). Smilagenin-induced neuritogenesis is partially inhibited by PD98059, a MEK1/2 inhibitor, suggesting that the neurotrophic effects of smilagenin are partially mediated through MEK1/2. This inhibition experiment is described below and the results are shown in Table 20 below.

Cortical neurones were cultured as detailed above. Neurones were exposed to vehicle (DMSO, 0.25%) or PD98059 (10 μM) for 1 h. After 1 h, vehicle, smilagenin (30 nM) or BDNF (1.85 nM) was added to the medium in the maintained presence of PD98059. Following 24 h exposure to smilagenin (30 nM), vehicle (DMSO, 0.25%) or BDNF (1.85 nM), the neurones were washed using PBS and fixed in glutaraldehyde (2.5%) in PBS. Photographs of 40-60 neurones expressing neurites were taken with a camera fixed on a microscope (objective ×20, Nikon). The neurite length was measured by an analysis of the photographs.

The results are shown in Table 20 below.

TABLE 20
Inhibition of smilagenin-induced neurite outgrowth
of rat primary cortical neurones
Cortical neurones

| | Neurite length |
|---|---|
| Condition | (% of control) |
| Control | 100 ± 3.18 |
| Control with PD98059 (10 μM) | 96.08 ± 2.47 |
| Smilagenin (30 nM) | 146.78 ± 6.75⁺⁺⁺⁺ |
| Smilagenin (30 nM) with PD98059 (10 μM) | 120.27 ± 5.80**** |
| BDNF (1.85 nM) | 125.30 ± 5.80⁺⁺⁺⁺ |
| BDNF (1.85 nM) with PD98059 (10 μM) | 99.07 ± 5.15**** |

Mean ± s.e.mean; n = 86-109 neurones per culture, n = 2 were used, statistical analysis performed using one-way ANOVA followed by Fisher's post-hoc test.
⁺⁺⁺⁺= p < 0.001 compared to control;
****= p < 0.001 compared to the same condition without PD98059

A similar experiment was performed using sarsasapogenin that produced similar results.

The cAMP response element binding protein (CREB) belongs to a family of transcription factors and is important in regulating neuronal survival. In addition, following trk receptor activation CREB is upregulated (Finkbeiner, Neuron, 2000, 25, pp. 11-14). Sarsasapogenin significantly increased the amount of phosphorylated CREB (pCREB, the active form of CREB) in Chinese hamster ovary (CHO) cells. This experiment is described below and the results are shown in Table 21 below.

The CHO were incubated with DMSO (0.5%) or sarsasapogenin (10 μM) for 24 h. The cells were then washed with cold PBS, lysed in sodium dodecyl sulfate (SDS) buffer, boiled for 5 min and the protein content measured by the Bradford method. The samples were then separated on SDS polyacrylamide gels and transferred to PVDF (Bio-Rad) membrane. After exposure for 1 h in 5% skimmed milk powder, membranes were incubated overnight at 4° C. in primary antibody: mouse pCREB (Upsdate, 1:1000) and mouse β-actin (Santa Cruz, 1:1000). The membranes were then incubated in peroxidase conjugated secondary antibody (Wuhan Boster Biology Technology, China. 1:2000) for 1 h at room temperature and developed with ECL reagents (Pierce). Membranes were stripped by incubating in 2-mercaptoethanol (100 mM), SDS (2%), Tris HCl (62.5 mM) at pH 6.8 and 50° C. for 30 min Densitometric quantification of immunostaining was performed using Image J analysis system with an image analyzer (Gel Doc 2000, Bio-Rad). The relative amount of immunostaining of each band of pCREB was normalised to the β-actin band run in the same experiment and expressed as arbitrary units.

TABLE 21
Expression of phosphorylated CREB following 24 h
exposure to sarsasapogenin in CHO cells
Phosphorylated CREB (Arbitary units)

| Control | Sarsasapogenin (10 μM) |
|---|---|
| 0.729 ± 0.112 | 1.342 ± 0.084⁺⁺ |

Mean ± s.e.mean; n = 5, statistical analysis was performed by paired t-test,
⁺⁺= p < 0.01 compared to control.

### Example 10

### Pre-Treatment with Sarsasapogenin, Smilagenin, Episarsasapogenin and Epismilagenin Reduces Glutamate-Induced Damage to Cortical Neurones

Exposure of rat primary cortical neurones to glutamate increases lactate dehydrogenase (LDH) activity measured 24 h after glutamate exposure, indicating significant neuronal damage. Rat cortical neurones were cultured by modification of a method previously described (Singer, et al., Neuroscience Letters, 1996, 212, pp. 13-16). On day 10 of culture, the medium was changed to a serum-free defined medium. On day 12, the cultures were washed and placed for 24 h in fresh medium containing test compound or vehicle (DMSO, 0.25%). On day 13 neurones were exposed to glutamate (100 μM; 10 min) at 37° C. The cultures were then washed with, and placed in, fresh medium supplemented with test compound or vehicle for a further 24 h before LDH was measured. Neuronal damage was assessed by measuring LDH activity in the media at 24 h after glutamate exposure.

The results are shown in Tables 22 to 26 below.

TABLE 22
Sarsasapogenin reduces glutamate-induced
damage in cortical neurones

| Cortical neurones | Neuronal survival |
|---|---|
| Conditions | (% of control) |
| Control | 100.00 ± 2.23 |
| Glutamate (100 μM) | 65.83 ± 2.46⁺⁺⁺ |
| Glutamate + sarsasapogenin (1 nM) | 76.88 ± 2.79*** |
| Glutamate + sarsasapogenin (3 nM) | 77.23 ± 2.62*** |
| Glutamate + sarsasapogenin (10 nM) | 73.50 ± 3.05* |
| Glutamate + sarsasapogenin (30 nM) | 78.91 ± 2.97*** |
| Glutamate + sarsasapogenin (100 nM) | 76.30 ± 4.15*** |

Mean ± s.e.mean; n = 4 wells per culture, 3 cultures were used, statistical analysis performed using ANOVA followed by Fisher's post-hoc test.
⁺⁺⁺= p < 0.005, compared with control
*= p < 0.05;
**= p < 0.01;
***= p < 0.005; compared with glutamate

TABLE 23
Smilagenin reduces glutamate-induced damage in cortical neurones

| Cortical neurones | Neuronal survival |
|---|---|
| Conditions | (% of control) |
| Control | 100.00 ± 4.17 |
| Glutamate | 67.09 ± 3.46 ⁺⁺⁺ |
| Glutamate + smilagenin (1 nM) | 81.53 ± 1.66 *** |
| Glutamate + smilagenin (3 nM) | 78.19 ± 1.85 ** |
| Glutamate + smilagenin (10 nM) | 82.50 ± 1.00 *** |
| Glutamate + smilagenin (30 nM) | 89.86 ± 3.55 *** |
| Glutamate + smilagenin (100 nM) | 82.45 ± 2.18 *** |

Mean ± s.e.mean; n = 4 wells, 1 culture was used

TABLE 24
Episarsasapogenin reduces glutamate-
induced damage in cortical neurones

| Cortical neurones | Neuronal survival |
|---|---|
| Conditions | (% of control) |
| Control | 100.00 ± 4.17 |
| Glutamate | 67.09 ± 3.46 ⁺⁺⁺ |
| Glutamate + episarsasapogenin (1 nM) | 84.79 ± 2.40 *** |
| Glutamate + episarsasapogenin (3 nM) | 80.39 ± 5.18 * |
| Glutamate + episarsasapogenin (10 nM) | 83.80 ± 4.18 *** |
| Glutamate + episarsasapogenin (30 nM) | 87.17 ± 2.51 *** |
| Glutamate + episarsasapogenin (100 nM) | 86.42 ± 2.95 *** |

Mean ± s.e.mean; n = 4 wells, 1 culture was used

TABLE 25
Epismilagenin reduces glutamate-
induced damage in cortical neurones

| Cortical neurones | Neuronal survival |
|---|---|
| Conditions | (% of control) |
| Control | 100.00 ± 5.18 |
| Glutamate | 70.15 ± 1.07 ⁺⁺⁺ |
| Glutamate + epismilagenin (3 nM) | 82.49 ± 3.93 ** |
| Glutamate + epismilagenin (10 nM) | 78.57 ± 2.15 |
| Glutamate + epismilagenin (30 nM) | 81.76 ± 2.09 ** |
| Glutamate + epismilagenin (100 nM) | 78.39 ± 1.75 |
| Glutamate + epismilagenin (300 nM) | 78.86 ± 1.80 * |

Mean ± s.e.mean; n = 4 wells, 1 culture was used

TABLE 26
Diosgenin does not reduce glutamate-
induced damage in cortical neurones

| Cortical neurones | Neuronal survival |
|---|---|
| Conditions | (% of control) |
| Control | 100.00 ± 4.20 |
| Glutamate | 67.67 ± 4.54 ⁺⁺⁺ |
| Glutamate + diosgenin (3 nM) | 69.43 ± 1.76 |
| Glutamate + diosgenin (10 nM) | 66.51 ± 5.13 |
| Glutamate + diosgenin (30 nM) | 68.98 ± 5.39 |
| Glutamate + diosgenin (100 nM) | 70.95 ± 5.03 |
| Glutamate + diosgenin (300 nM) | 75.02 ± 2.68 |

Mean ± s.e.mean; n = 4 wells, 1 culture was used

In rat primary cortical neurones, pre-treatment with sarsasapogenin, smilagenin, episarsasapogenin (1-100 nM) and epismilagenin (3-300 nM), 24 h prior to glutamate exposure, significantly reduced the glutamate-induced LDH release compared to neurones exposed to glutamate alone.

By contrast, pre-treatment with diosgenin (3-300 nM), 24 h prior to glutamate exposure, did not prevent the neuronal damage.

The activity of sarsasapogenin, smilagenin, episarsasapogenin and epismilagenin reached a plateau at nanomolar concentration without causing any toxicity. The test compounds at micromolar concentrations in these experimental conditions precipitate out of solution.

This Example complements the in vitro experiments in Examples 2 to 4 of PCT Patent Application No. WO-A-03/082893, incorporated herein by reference. Those experiments demonstrated that pre-treatment of rat primary cortical neurones with sarsasapogenin, episarsasapogenin, smilagenin, epismilagenin or 3-ketones or 3-esters thereof significantly prevented or reversed glutamate-induced neurodegeneration, whereas diosgenin showed no such activity.

### Example 11

### Anti-Apoptotic Effect of Sarsasapogenin, Episarsasapogenin, Smilagenin and Epismilagenin in Dopaminergic Neurones

Rat dopaminergic neurones were cultured as previously described (Schinelli et al., Journal of Neurochemistry, 1988, 50, pp. 1900-1907). On day 5, the cultures were washed and placed in fresh medium containing test compounds (30 nM), vehicle (DMSO, 0.25%) or a combination of BDNF (1.85 nM) and GDNF (0.17 nM) for 24 h.

Exposure of rat primary dopaminergic neurones to MPP⁺ (2 μM, 24 h) causes a significant decrease in the number of dopaminergic neurones compared to the control. On day 6, MPP⁺ (2 μM) was added to the cultures in the presence of test compounds, vehicle or a combination of BDNF and GDNF for a further 48 h. MPP⁺ induces neuronal death, via inhibition of complex I in the mitochondria and consequent ATP depletion, resulting in the production of free radicals and induction of apoptosis. After the incubation period, the cultures were fixed with paraformaldehyde in PBS (4%). After fixation, the neurones were permeabilised with Triton ×100 (0.05%) for 30 min. The neurones were then incubated with anti-tyrosine hydroxylase (TH) at 37° C. for 2 h. The neurones were washed three times with PBS, and then incubated with goat anti mouse/Cy3 for 2 h at 37° C. The neurones were mounted and examined with the fluorescence microscopy.

The results are shown in Table 27 below.

TABLE 27
Sarsasapogenin, smilagenin, episarsasapogenin and epismilagenin
reduce MPP⁺-induced loss of mesencephalic dopaminergic neurones

| Dopaminergic neurones | Neuronal survival |
|---|---|
| Conditions | (% of control) |
| Control | 100.00 ± 3.20 |
| + MPP⁺ (2 μM) | 55.31 ± 3.15⁺⁺⁺⁺ |
| + MPP⁺ + sarsasapogenin (30 nM) | 88.73 ± 4.39**** |
| + MPP⁺ + smilagenin (30 nM) | 97.91 ± 3.63**** |
| + MPP⁺ + episarsasapogenin (30 nM) | 95.01 ± 4.52**** |
| + MPP⁺ + epismilagenin (30 nM) | 115.12 ± 4.73**** |
| + MPP⁺ + BDNF (1.85 nM) & GDNF (0.17 nM) | 121.94 ± 6.51**** |

Mean ± s.e.mean; n = 40 or 80 fields, wells per culture, n = 1 or 2 cultures were used. Statistical analysis was performed by ANOVA followed by Dunnett's post-hoc test;
⁺⁺⁺⁺= p < 0.005, compared with control,
****= p < 0.005; compared with MPP⁺

Sarsasapogenin, smilagenin, episarsasapogenin and epismilagenin significantly prevent MPP⁺-induced decrease in dopaminergic neurones. Exposure to a combination of neurotrophic factors, BDNF and GDNF also significantly prevents MPP⁺-induced decrease in dopaminergic neurones.

### Example 12

### Sarsasapogenin and Smilagenin are Neurorestorative after Glutamate or MPP⁺ Induced Damage

### Cortical Neurones

An important goal of treatment for neurodegenerative disorders is not only to prevent progression but also to reverse the neuronal loss that occurs in patients. Following exposure of rat primary cortical neurones to glutamate (100 μM; 10 min), sarsasapogenin and smilagenin (30 nM) significantly reversed the glutamate-induced damage 24 h post-treatment. This Example develops the work reported in Examples 2 to 4 of PCT patent application No. WO-A-03/082893.

The rat cortical neurones were prepared as detailed above. On day 13, cultures were exposed to glutamate (100 μM) for 10 min at 37° C. in 5% CO₂/95% air atmosphere in defined medium. Following the incubation period, the cultures were washed and maintained in fresh medium, containing sarsasapogenin, smilagenin or vehicle. Cells were cultured for a further 24 h after glutamate exposure and were then assessed for neuronal damage as detailed above.

The results are shown in Table 28 below.

TABLE 28
Sarsasapogenin and smilagenin reverse glutamate-
induced damage in cortical neurones

| Cortical neurones | Neuronal survival |
|---|---|
| Condition | (% of control) |
| Control | 100.00 ± 4.03 |
| + Glutamate (100 μM) | 66.32 ± 2.36⁺⁺⁺⁺ |
| + Glutamate + sarsasapogenin (30 nM) | 103.43 ± 5.10**** |
| + Glutamate + smilagenin (30 nM) | 111.06 ± 3.40**** |

Mean ± s.e.mean; n = 4 wells per culture, 2 cultures were used, statistical analysis performed using one-way ANOVA followed by Fisher's post-hoc test;
⁺⁺⁺⁺= p < 0.001, compared with control,
****= p < 0.001 compared with glutamate

### Spinal Motor Neurones

The rat spinal motor neurones were prepared as detailed above. On day 10, the medium was removed and the cultures exposed to glutamate (4 μM) for 10 min at 37° C. in 5% CO₂/95% air atmosphere in defined medium. After the glutamate exposure, cultures were washed with DMEM at 37° C. then placed in fresh culture medium containing sarsasapogenin, smilagenin, vehicle or BDNF. After 48 h, the extent of motor neurone damage was determined as detailed above. This Example develops the work reported in Example 8 of PCT patent application No. WO-A-03/082893.

The results are shown in Table 29 below.

TABLE 29
Sarsasapogenin and smilagenin reverse glutamate-
induced damage in spinal motor neurones

| Spinal motor neurones | Neuronal survival |
|---|---|
| Condition | (% of control) |
| Control | 100.00 ± 8.87 |
| + Glutamate (4 μM) | 75.52 ± 2.58⁺ |
| + Glutamate + sarsasapogenin (0.03 nM) | 101.09 ± 4.12**** |
| + Glutamate + sarsasapogenin (3 nM) | 108.10 ± 3.56**** |
| + Glutamate + sarsasapogenin (300 nM) | 120.43 ± 7.46**** |
| + Glutamate + smilagenin (0.03 nM) | 90.98 ± 2.46* |
| + Glutamate + smilagenin (3 nM) | 101.53 ± 3.18**** |
| + Glutamate + smilagenin (300 nM) | 106.61 ± 4.24**** |
| + Glutamate + BDNF (3 nM) | 106.60 ± 6.14**** |

Mean ± s.e.mean; n = 6 wells per cultures, 2 cultures and n = 1 culture for BDNF were used, statistical analysis performed using a one-way ANOVA followed by Fisher's post-hoc test.
⁺= p < 0.005 compared with control;
****= p < 0.001,
*= p < 0.05, compared with glutamate

Exposure of rat primary spinal motor neurones to glutamate (4 μM; 10 min) increased LDH activity measured 48 h after glutamate exposure, indicating significant neuronal damage. Sarsasapogenin and smilagenin (0.03-300 nM) significantly reversed glutamate-induced damage 48 h post-treatment.

However, the reversal of damage provided by the lowest concentration of sarsasapogenin and smilagenin varied between cultures, suggesting that 0.03 nM may be at the lower limit of activity in this model. Brain derived neurotrophic factor (3 nM) was used as a positive control and significantly reversed glutamate-induced LDH activity when compared to spinal motor neurones exposed to glutamate alone.

### Dopaminergic Neurones

Rat primary dopaminergic neurones were prepared as described above. On day 5, MPP⁺ (2 μM) was added to the cultures for 24 h in culture medium at 37° C. in 5% CO₂/95% air atmosphere. Exposure of rat primary dopaminergic neurones to MPP⁺ (2 μM, 24 h) causes a significant decrease in the number of dopaminergic neurones compared to the control. On day 6 the medium was removed and fresh medium containing vehicle (DMSO, 0.25%), sarsasapogenin, smilagenin or a combinations of BDNF and GDNF was added. After 48 h, the extent of dopaminergic damage was determined as detailed above.

The results are shown in Table 30 below.

TABLE 30
Sarsasapogenin and smilagenin reverse
MPP⁺-induced damage in dopaminergic neurones

| Dopaminergic neurones | Neuronal survival |
|---|---|
| Condition | (% of control) |
| Control | 100.00 ± 5.79 |
| + MPP⁺ (2 μM) | 75.81 ± 4.00⁺⁺⁺ |
| + MPP⁺ + sarsasapogenin (30 nM) | 104.30 ± 5.63**** |
| + MPP⁺ + smilagenin (30 nM) | 113.44 ± 4.62**** |
| + MPP⁺ + BDNF (1.85 nM) & | 97.85 ± 4.68*** |
| GDNF (0.17 nM) | |

Mean ± s.e.mean; n = 40 fields n = 1 culture was used, statistical analysis performed using one-way ANOVA followed by Fisher's post-hoc test.
⁺⁺⁺= p < 0.005 compared with control;
***= p < 0.005;
****= p < 0.001, compared with MPP⁺

The data show that exposure to sarsasapogenin and smilagenin (30 nM) significantly reversed MPP⁺-induced decrease in dopaminergic neurones. Exposure to a combination of neurotrophic factors, BDNF (1.85 nM) and GDNF (0.17 nM) also significantly reversed the MPP⁺-induced decrease in dopaminergic neurones.

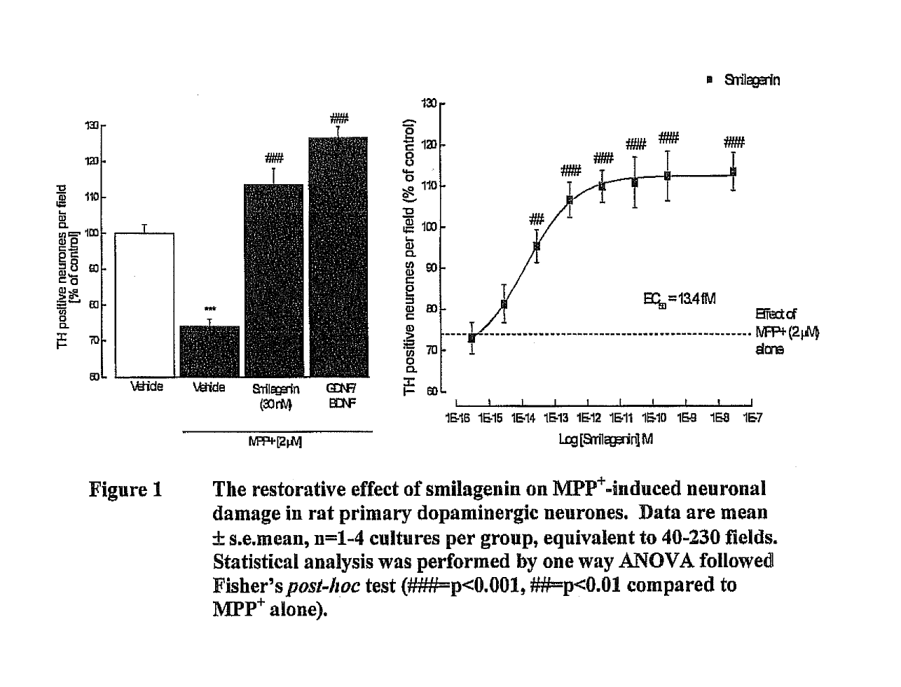

In similar experiments, the results of which are shown in FIG. 1, the effect of different concentrations of smilagenin to reverse MPP⁺ (2 μM, 24 h) induced neuronal damage in rat primary dopaminergic neurones was examined Concentrations of BDNF, GDNF and vehicle were as stated above. The dopaminergic cultures were incubated in the medium containing smilagenin (0.3 fM to 30 nM), a combination of BDNF (1.85 nM) and GDNF (0.17 nM) or vehicle (DMSO, 0.25%) for 24 h. MPP⁺ (2 μM) or vehicle was then added to the medium, and the cultures were incubated for a further 48 h. The number of dopaminergic (TH-positive) neurones per field was quantified by immunohistochemistry and fluorescence microscopy and then normalised to its own control so that the data could be combined. The 48 h treatment with smilagenin (3 fM-30 nM) after 24 h exposure to MPP⁺ significantly reversed the MPP⁺-induced neuronal damage with an EC₅₀ of 13.4 fM.

### Example 13

### Oral Administration of Sarsasapogenin and Smilagenin Improve Recovery of Nerve Function in a Mouse Model of Nerve Damage (pmn Mice)

The progressive motor neuropathy (pmn) mouse is a genetic model of a degenerative motor neurone disease, involving a dying-back process with distal axon degeneration and relative preservation of proximal axons and cell bodies (Schmalbruch et al., Journal of Neuropathology and Experimental Neurology, 1991, 50, pp. 192-204). The pmn/pmn homozygous suffer caudio-cranial degeneration of motor axons and die a few weeks after birth, probably due to respiratory muscle denervation (Schmalbruch et al., Journal of Neuropathology and Experimental Neurology, 1991, 50, pp. 192-204; Sendtner et al., Nature, 1992, 358, pp. 502-504). Although the pmn mouse cannot be considered as an exact animal model of any counterpart of any particular human motor neurone disease (Kennel et al., Neurobiology of Disease, 1996, 3, pp. 137-147), it represents a useful model to evaluate the potential of new drug candidates for neurodegenerative diseases. This mouse model has already been used to determine the pathogenic mechanisms underlying motor neurone degeneration (Sagot et al., Journal of Neuroscience, 1995, 15, pp. 7727-7733) and to evaluate potential therapeutic strategies for the treatment of motor neurone diseases (Haase et al., Nature Medicine, 1997, 3, pp. 429-436; Sendtner et al., Nature, 1992, 358, pp. 502-504; Sagot et al., Journal of Neuroscience, 1995, 15, pp. 7727-7733; Sagot et al., Journal of Neuroscience, 1996, 16, pp. 2335-2341) such as ALS, progressive muscular atrophy, spinal muscular atrophy, progressive bulbar palsy, pseudobulbar palsy and primary lateral sclerosis. This Example develops the work reported in Example 11 of PCT patent application No. WO-A-03/082893, incorporated herein by reference.

Affected homozygous +/+ pmn (“pmn mice”) mice were obtained from a breeding colony of extra toe locus(Xt)+/+pmn double heterozygous mice maintained at Neurofit (Illkirch, France). The pmn mice were dosed by oral gavage every day, starting 10 days after birth, just after the initial symptoms of the disease manifest. Sarsasapogenin (0.03, 0.3 and 3 μg/kg/day) was administered to pmn mice as a suspension in oil (10 ml/kg). Electromyographic (EMG) recordings were performed using a standard Neuromatic 2000M electromyograph apparatus in accordance with the guidelines of the American Association of Electrodiagnostic Medicine. Standard behavioural tests (grid, rotarod and hanging tests) performed weekly from day 8 assessed the motor performances of the pmn mice.

The effect of sarsasapogenin on motor function was assessed by recording the amplitude of gastrocnemius evoked motor response (CMAP, an indirect measurement of the number of functional motor neurones).

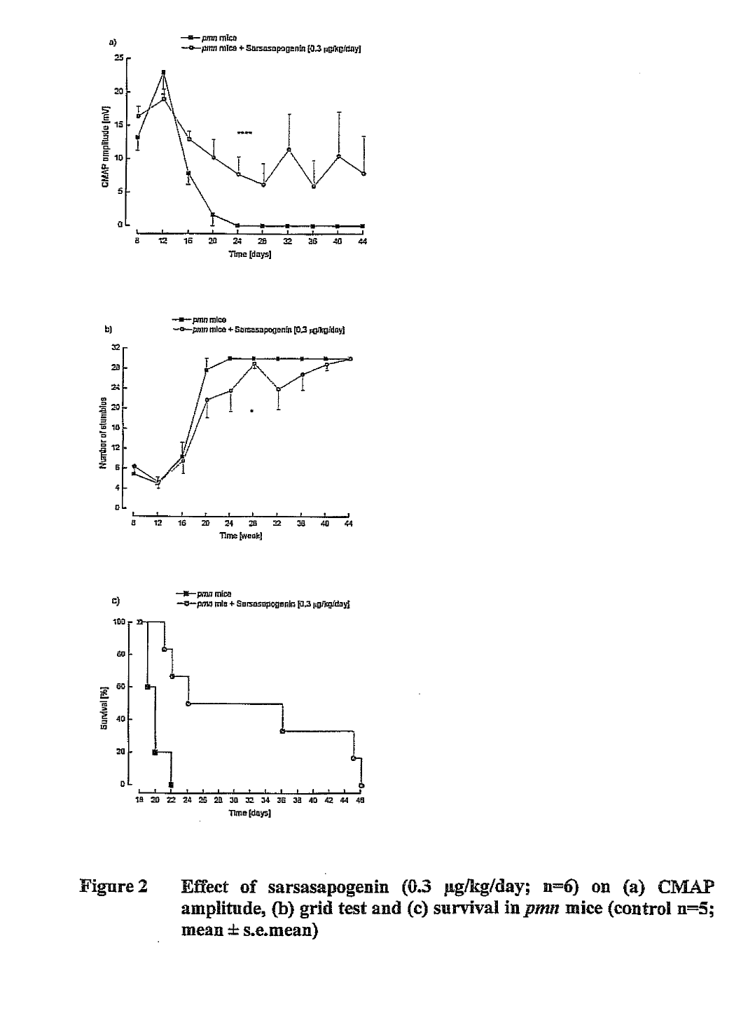

The results are shown in FIG. 2 of the drawings.

The pmn control group showed a rapid decline in the amplitude of CMAP at 12 days of age. Daily oral administration of sarsasapogenin (0.3 μg/kg/day) to pmn mice delayed the deterioration of motor function (p<0.001). The number of stumbles made by control mice increased rapidly from 12 days of age. Daily oral administration of sarsasapogenin (0.3 μg/kg/day) to pmn mice significantly delayed the deterioration in the rotarod and grid test performances compared to the pmn control group (p=0.02 MANOVA analysis). Daily oral administration of sarsasapogenin (0.3 μg/kg/day) significantly increased the survival of pmn mice compared to the pmn control group (up to 62% compared to control, log rank, χ²=7.36, p=0.006).

By contrast, a daily oral administration of sarsasapogenin at the lowest tested dosage (0.03 μg/kg/day), following the onset of the clinical symptoms, does not delay the progression of the motor neurone degeneration in this genetic model.

These results suggest that sarsasapogenin, an orally active, non-peptide neurotrophic factor inducer, is able to delay the progression of the motor neurone degeneration in this genetic model. Neurotrophic factors (ciliary neurotrophic factor; CNTF; Sagot et al., Journal of Neuroscience, 1995, 15, pp. 7727-7733) and GDNF (Sagot et al., Journal of Neuroscience, 1996, 16, pp. 2335-2341) have been tested in the pmn mouse model. These studies showed that CNTF (i.p. administration of CNTF-secreting cells) increased the survival time by 40% and improved motor function, whereas GDNF improved the motor neurone survival but did not slow down the disease (Sagot et al., Journal of Neuroscience, 1996, 16, pp. 2335-2341). Neurotrophic factors have been considered as a possible treatment for motor neurone diseases; however, as proteins their widespread clinical use is highly problematic. The non-peptide neurotrophic compound SR 57746A (Xaliproden), orally administered to pmn mice from birth, delayed the progress of the motor neurodegeneration improving the mouse motor performances and lifespan (˜50%; Duong et al., British Journal of Pharmacology, 1998, 124, pp. 811-817). Furthermore, CGP 3466B (an anti-apoptotic agent) orally administered at the onset of the disease delayed the progression of the disease and improved the pmn mouse lifespan by 57% (Sagot et al., British Journal of Pharmacology, 2000, 131, pp. 721-'728); the molecule BN 80933 (an inhibitor neuronal nitric oxide synthase and lipid peroxidation) improved the pmn mouse lifespan by 40% (Sagot et al., British Journal of Pharmacology, 2000, 131, pp. 721-728).

Importantly, sarsasapogenin, when orally administered after the symptoms of the disease manifest, delayed the progression of the disease and improved the pmn mouse lifespan in this model in vivo by up to 62%. Similar results were obtained with smilagenin.

### Example 14

### Oral Administration of Sarsasapogenin and Smilagenin Improve Recovery of Nerve Function in a Further Mouse Model of Nerve Damage (Nerve Crush Model)

The sciatic nerve crush model is a well characterised reversible model for motor neurone disease and post-traumatic nerve injuries (McMahon and Priestley, Current Opinion in Neurobiology, 1995, 5, pp. 616-624). The nerve damage is produced by mechanical pressure using haemostatic forceps, applied twice, 5 mm proximal to the trifurcation of the right sciatic nerve of these mice. This results in nerve degeneration over a two-week period followed by localised inflammation of the nerve that lasts for up to four weeks. The loss of nerve function recovers progressively over a 4-5 week period after the mechanical insult.

Following sciatic nerve damage, daily oral administration of sarsasapogenin (3 mg/kg/day, oral gavage in oil) and smilagenin (0.3 and 3 mg/kg/day, oral gavage in oil) for 6 weeks to the C57 mice significantly improved the recovery of nerve function as measured by CMAP parameters in the gastrocnemius muscle (amplitude, latency and duration, indirect markers of active motor fibres, motor nerve conduction velocity and functionality of nerve fibres, respectively) and morphological analysis of the sciatic nerve (proportion of degenerated fibres). 4-Methylcatechol (10 μg/kg/day, i.p.) was used as a positive control (Kaechi et al., Journal of Phamacology and Experimental Therapeutics, 1995, 272, pp. 1300-1304).

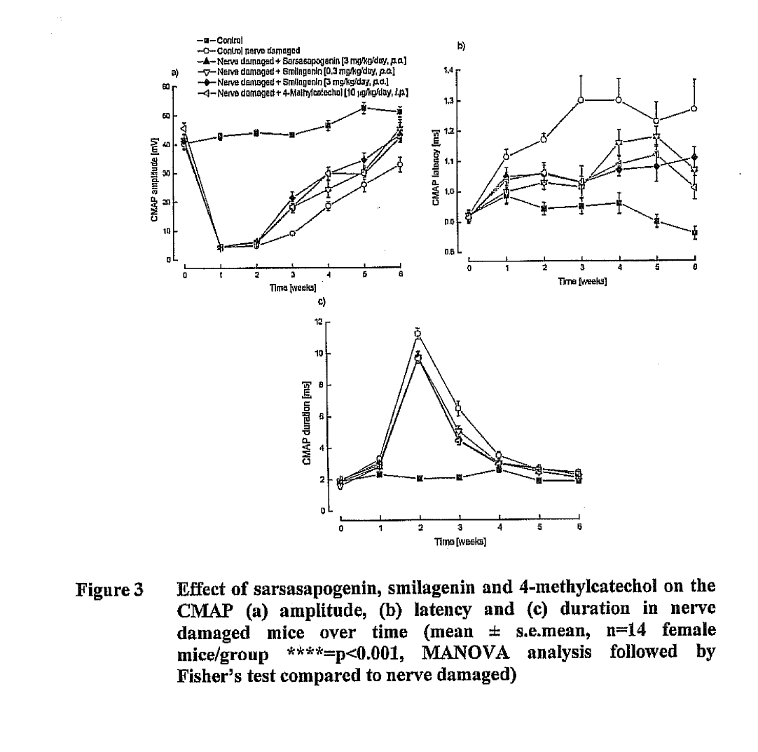

The results are shown in FIG. 3 of the drawings.

C57b1/6 RJ mice were anaesthetised with ketamine chlorhydrate (60 mg/kg, i.p.). The sciatic nerve was surgically exposed at mid thigh level and crushed at 5 mm proximal to the trifurcation of the sciatic nerve. The nerve was crushed twice for 30 s with a haemostatic forceps with a 90-degree rotation between each crush. This resulted in nerve degeneration over a two-week period followed by localised inflammation of the nerve that lasted for up to four weeks. The loss of nerve function recovered progressively over a 4-5 week period after mechanical insult. Electromyographic recordings were assessed as described above.

The results are shown in Table 31 below.

TABLE 31
Sarsasapogenin and smilagenin decrease the number of
degenerated fibres in a mouse model of nerve damage

| | Degenerated fibres |
|---|---|
| Groups | (% of control) |
| Control | 0.00 ± 6.71 |
| Nerve crush | 109.10 ± 2.65⁺⁺⁺⁺ |
| Sarsasapogenin (3 mg/kg/day) | 33.50 ± 12.60**** |
| Smilagenin (0.3 mg/kg/day) | 16.70 ± 4.23**** |
| Smilagenin (3 mg/kg/day) | −8.10 ± 6.03**** |
| 4-Methylcatechol (10 μg/kg/day) | −7.48 ± 1.62**** |

Mean ± s.e.mean. Statistical analysis on the degenerated fibres was performed using a one-way ANOVA and Dunnett's post-hoc test, n = 3-4.
⁺⁺⁺⁺=p < 0.001, compared to control;
****= p < 0.001, compared to nerve crush

Sarsasapogenin and smilagenin are orally active and are able to improve the recovery of nerve function and stimulate the nerve regeneration in the sciatic nerve crush model.

### Example 15

### Sarsasapogenin and Smilagenin Reduce Anxiety and Restore Cognitive Ability and the Decline in BDNF in Aged Animals

Old Sprague Dawley (SD) rats (20 month old) were orally administered sarsasapogenin or smilagenin (18 mg/kg/day) for 3 months. Young SD rats (4 month old) were used as healthy positive control and old SD rats (20 month old) were used as neurodegenerative control. A Y-maze was used to assess learning and memory, and was considered also a model of anxiety in light of the nature of the test that caused distress to the animal. On the floor of each arm of the Y-maze was an array of copper rods (2 mm×140 mm) to which an adjustable voltage electric current was applied when needed. Each arm was 450 mm long with a 15 W lamp at the end. Following 2 months of treatment each rat was trained for 7 consecutive days, once on each day. During each training session, a rat was put into one arm of the Y-maze and after 2 min an electrical current applied to the copper rods in the anti-clockwise arm and the lamp of the clockwise arm was illuminated, indicating the non-electrified area. If the rat went into the illuminated arm a correct response was recorded, otherwise a wrong response was recorded. This stimulation-response test was repeated 20 times each day, with a pause of 5 s between each test. The number of correct responses and the total time period for the 20 tests were recorded. A quotient of the number of correct responses divided by the total response time was calculated and used as an index for learning ability, with the higher the quotient the greater the learning ability. One month after the learning test (3 months of treatment) the Y-maze test was carried out again and the quotient obtained was used as an index for memory ability. At the end of the treatment the rats were killed and the brains removed for quantification of BDNF using an ELISA (data of BDNF presented in Example 3 above).

The results of the Y-maze experiment are shown in Table 32 below.

TABLE 32
Sarsasapogenin and smilagenin restore the cognitive ability (learning
and memory) and the decline of BDNF levels in aged rats

| | Learning ability | Memory ability |
|---|---|---|
| | (correct response/ | (correct response/ |
| Groups | total time) | total time) |
| Young | 5.97 ± 0.35 | 5.27 ± 0.35 |
| Aged | 2.39 ± 0.26⁺⁺⁺⁺ | 2.16 ± 0.30⁺⁺⁺⁺ |
| Aged + sarsasapogenin | 5.02 ± 0.50**** | 4.66 ± 0.34**** |
| (18 mg/kg/day) | | |
| Aged + smilagenin | 4.81 ± 0.32**** | 4.58 ± 0.26**** |
| (18 mg/kg/day) | | |

Mean ± s.e.mean; n = 9-10, statistical analysis performed using paired one tailed Student's t test
⁺⁺⁺⁺=p < 0.001 compared to young rats;
****= p < 0.001, compared to aged rats

Sarsasapogenin and smilagenin (18 mg/kg/day), orally administered to aged rats for up to 3 months, reduce the anxiety, restore the cognitive ability (learning and memory ability) towards that observed in the young rats.

### Example 16

### Orally Administered Sarsasapogenin and Smilagenin are Delivered to a Range of Body Tissues

Sarsasapogenin and smilagenin have been demonstrated to be orally active and their plasma, brain, spinal cord (and other tissue) concentrations have been measured following oral administration in rodents and non-rodents.

The results are shown in Table 33 below.

TABLE 33
Sarsasapogenin and smilagenin distribute to the plasma, brain and
spinal cord following a single oral administration

| | | | | | Concentration of compound | | |
|---|---|---|---|---|---|---|---|
| | | | | | (ng equivalents/g tissue) | | |
| | | | Dose | | | | Spinal |
| Compound | Species | Sex | (mg/kg) | Time (h) | Plasma | Brain | cord |
| Sarsasapogenin | Rat | M | 30 | 1 | 1690 | 1140 | 1090 |
| Sarsasapogenin | | M | 30 | 4 | 2920 | 4970 | 4350 |
| Sarsasapogenin | | M | 30 | 8 | 2970 | 6920 | 6060 |
| Sarsasapogenin | | M | 30 | 24 | 967 | 3800 | 4720 |
| Sarsasapogenin | | M | 30 | 168 | 149 | 194 | 747 |
| Sarsasapogenin | | F | 30 | 1 | 1530 | 1210 | 3020 |
| Sarsasapogenin | | F | 30 | 4 | 2050 | 3860 | 3830 |
| Sarsasapogenin | | F | 30 | 8 | 1760 | 5980 | 5080 |
| Sarsasapogenin | | F | 30 | 24 | 629 | 2800 | 3290 |
| Sarsasapogenin | | F | 30 | 168 | 90 | 134 | 590 |
| Sarsasapogenin | Dog | M | 25 | 4 | 0.641 | 1.37 | 1.13 |
| Sarsasapogenin | | M | 25 | 24 | 0.196 | 2.33 | 1.82 |
| Sarsasapogenin | | M | 25 | 168 | 0.309 | 0.605 | 1.49 |
| Sarsasapogenin | | F | 25 | 4 | 0.184 | 0.128 | 0.515 |
| Sarsasapogenin | | F | 25 | 24 | 1.79 | 8.64 | 5.79 |
| Sarsasapogenin | | F | 25 | 168 | 0.115 | 0.535 | 1.43 |
| Smilagenin | Rat | M | 18 | 1 | 2045 | 769 | 958 |
| Smilagenin | | M | 18 | 4 | 3842 | 3372 | 3286 |
| Smilagenin | | M | 18 | 8 | 3896 | 4457 | 4520 |
| Smilagenin | | M | 18 | 24 | 857 | 1417 | 1957 |
| Smilagenin | | M | 18 | 168 | 122 | 122 | 256 |
| Smilagenin | | F | 18 | 1 | 1626 | 704 | 608 |
| Smilagenin | | F | 18 | 4 | 1824 | 2330 | 2049 |
| Smilagenin | | F | 18 | 8 | 1658 | 2940 | 2823 |
| Smilagenin | | F | 18 | 24 | 394 | 954 | 1284 |
| Smilagenin | | F | 18 | 168 | 46 | 52 | 243 |
| Smilagenin | Dog | M | 10 | 2 | 3878 | 3670 | 1805 |
| Smilagenin | | M | 10 | 24 | 1508 | 5813 | 3647 |
| Smilagenin | | M | 10 | 168 | 1129 | 1727 | 3165 |

Orally administered sarsasapogenin and smilagenin migrate to neuronal sites of the body and to blood plasma.

### Example 17

### Orally Administered Sarsasapogenin and Smilagenin are Non-Toxic at Effective Doses

Sarsasapogenin and smilagenin are active at nanomolar concentrations in vitro, while lower concentration are inactive or present a variable activity in neurones. At higher concentrations sarsasapogenin and smilagenin do not show the toxicity that is observed at higher concentration of neurotrophic factors in vitro.

Long term toxicity studies following oral administration of high doses have been performed with sarsasapogenin (up to 26 week in rats and 39 weeks in non-rodents) and smilagenin (up to 52 weeks in mice and non-rodents) without showing any signs of toxicity or adverse events that could appear once high level of neurotrophic factor are reached.

### Example 18

### Smilagenin Reduces Parkinsonism in MPTP-Lesioned Macaques and Modulates GDNF and BDNF Concentration in the Putamen

Nineteen female cynomolgus monkeys (Macaca fascicularis, 3.0-4.5 kg, 4-6 years old) were acclimatised to the experimental setting and procedures for 3 months and baseline behaviour was assessed in all animals. Fourteen female macaques received MPTP (0.2 mg/kg/day, s.c.) until marked, stable, parkinsonian symptoms developed. Animals (n=7/group) were randomly assigned to two groups; smilagenin (20 mg/kg/day, p.o.) or vehicle control (HPMC, 0.5% w/v containing Tween 80, 0.2% v/v). The 5 macaques that did not receive MPTP were administered vehicle and used as a control group.

Assessments of parkinsonian disability were made after MPTP administration and following 18 weeks of smilagenin or vehicle administration. Parkinsonian disability was evaluated by post hoc analysis of DVD-recordings by a neurologist blinded to the treatment. At the end of the study the brains were removed and the levels of unbound GDNF and BDNF in the putamen were measured using Multiplex ELISA/Aushon assays. The MPTP treated macaques used in this experiment thus provide an accepted model for Parkinson's disease and similar motor-sensory neurodegenerative conditions.

The results are shown in Table 34 below.

TABLE 34
Smilagenin improves behaviour and modulates putamen
levels of GDNF and BDNF in MPTP-lesioned macaques

| | Median parkinsonian | Median | Mean ± s.e.mean of | Mean ± s.e.mean of |
|---|---|---|---|---|
| | disability post- | parkinsonian | unbound GDNF | unbound BDNF |
| | MPTP | disability | level in the putamen | level in the putamen |
| Group | administration | at week 18 | (pg/mg protein) | (pg/mg protein) |
| Control | N/A | N/A | 9.52 ± 1.98 | 257.58 ± 50.69 |
| macaques | | | | |
| MPTP-lesioned | 50.33 | 41.67 | 15.33 ± 1.86 | 388.73 ± 27.51 |
| macaques | | | | |
| MPTP + | 47.17 | 27.00^{###} | 5.09 ± 1.35** | 208.27 ± 36.58** |
| smilagenin | | | | |
| macaques | | | | |

###= p < 0.001 compared to parkinsonian disability post-MPTP administration. Statistical analysis performed using a non-matched 2-way-ANOVA with Bonferroni multiple comparison post hoc test.
**= p < 0.01 compared to MPTP-lesioned macaques. Statistical analysis performed using one-way ANOVA, followed by Tukey's post-hoc multiple comparison test.

Smilagenin, orally administered to MPTP-lesioned macaques for 18 weeks, significantly reduced the level of parkinsonism in MPTP-lesioned macaques. Smilagenin also significantly reduced the level of GDNF and BDNF in the putamen of MPTP-lesioned macaques compared to macaques receiving vehicle to a level not significantly different from that observed in control, unlesioned macaques.

This data indicates that the effect of smilagenin on GDNF and BDNF expression is a normalising effect under long term administration, i.e. that there is a long term regulatory effect protecting the animals against overexposure to GDNF and BDNF, by restoring levels to approximately the normal state. This change is importantly associated with a significant reduction in the level of parkinsonism in the macaques.

### DISCUSSION

The above examples demonstrate that the A/B-cis spirostane steroidal sapogenins sarsasapogenin and smilagenin are neurotrophic factor inducers as demonstrated by in vitro and ex vivo data; they are neuroprotective and neurorestorative in vitro and in vivo following oral administration. They do not require the presence of neurotrophic factors to function as inducers, and so appear to be true NF inducers, rather than NF enhancers.

Administration of neurotrophic factors (e.g. BDNF and GDNF) has been a strategy for disease modification in depression, schizophrenia, Parkinson's disease and other disorders, and this strategy has a strong scientific rationale. However, it has proved difficult to translate the scientific rationale to the clinic. This results from the protein nature of neurotrophic factors and the difficulties inherent in surgical, viral vector and cell-based gene/protein delivery approaches. The complex trophic requirements of neurones potentially limit the efficacy achieved by a single factor and the amount of neurotrophic factor required and the duration of treatment needed to achieve clinical benefit is also currently unknown.

The orally active neurotrophic factor inducers sarsasapogenin and smilagenin, and the related molecules as defined in this application, overcome many of those difficulties. We have shown here that sarsasapogenin and smilagenin are active at pico- and nanomolar concentrations in vitro. They do not show the toxicity that is observed at higher concentration of neurotrophic factor in vitro.

The evidence in the present application, taken together with the evidence previously published in the references referred to herein, shows that inducing self-regulated homeostasis of NFs, for example BDNF and/or GDNF, with limited and manageable side-effects, will be achieved by administering to the subject an effective amount of at least one agent selected from A/B-cis furostane, furostene, spirostane and spirostene steroidal sapogenins and ester, ether, ketone and glycosylated forms thereof, and that this will provide novel and unexpected benefits in a range of therapeutic and non-therapeutic methods for treating and preventing NF-mediated disorders and conditions such as neurological, psychiatric, inflammatory, allergic, immune, neoplastic and related conditions.

The foregoing broadly describes the present invention without limitation. Variations and modifications as will be readily apparent to those skilled in this art are intended to be included within the scope of the invention as defined in the appended claims.

1. A method of inducing self-regulated homeostasis of neurotrophic factors (NFs), for example BDNF and/or GDNF, in a subject, by modulating the subject's native NFs in a non-toxic manner under homeostatic control, the method comprising administering to the subject an effective amount of one or more agent selected from A/B-cis furostane, furostene, spirostane and spirostene steroidal sapogenins and ester, ether, ketone and glycosylated forms thereof.

2. A method according to claim 1, wherein the induction of self-regulated homeostasis of NFs takes place with limited and manageable side effects related to overinduction, overstimulation or overenhancement of NFs, for example NGF, and side effects related to receptor (ant)agonist action and side effects related to enzyme binding action.

3. A method according to claim 1, wherein the method is used in conjunction with a method for the treatment or prevention of NF-mediated disorders, for example selected from: (a) the treatment or prevention of a neurological disorder, for example selected from: dementia, age-related cognitive impairment, Alzheimer's disease, senile dementia of the Alzheimer's type (SDAT), Lewy body dementia, vascular dementia, Parkinson's disease, postencephalitic Parkinsonism, parkinsonism having a cause other than postencephalitic and other than Parkinson's disease, muscular dystrophy including facioscapulohumeral muscular dystrophy (FSH), Duchenne muscular dystrophy, Becker muscular dystrophy and Brace's muscular dystrophy, Fuchs' dystrophy, myotonic dystrophy, corneal dystrophy, reflex sympathetic dystrophy syndrome (RSDSA), neurovascular dystrophy, myasthenia gravis, Lambert Eaton disease, Huntington's disease, motor neurone diseases including amyotrophic lateral sclerosis (ALS), infantile spinal amyotrophy, multiple sclerosis, postural hypotension, pain, neuralgia, traumatic neurodegeneration e.g. following stroke or following an accident (for example, traumatic head or brain injury or spinal cord injury), Batten's disease, Cockayne syndrome, Down syndrome, corticobasal ganglionic degeneration, multiple system atrophy, cerebral atrophy, olivopontocerebellar atrophy, dentatorabral atrophy, pallidoluysian atrophy, spinobulbar atrophy, optic neuritis, sclerosing pan-encephalitis (SSPE), attention deficit disorder, post-viral encephalitis, post-poliomyelitis syndrome, Fahr's syndrome, Joubert syndrome, Guillain-Barre syndrome, lissencephaly, Moyamoya disease, neuronal migration disorders, autistic syndrome, polyglutamine disease, Niemann-Pick disease, progressive multifocal leukoencephalopathy, pseudotumor cerebri, Refsum disease, Zellweger syndrome, supranuclear palsy, Friedreich's ataxia, spinocerebellar ataxia type 2, Rhett syndrome, Shy-Drager syndrome, tuberous sclerosis, Pick's disease, chronic fatigue syndrome, neuropathies including hereditary neuropathy, diabetic neuropathy and mitotic neuropathy, prion-based neurodegeneration, including Creutzfeldt-Jakob disease (CJD), variant CJD, new variant CJD, bovine spongiform encephalopathy (BSE), GSS, FFI, kuru and Alper's syndrome, Joseph's disease, acute disseminated encephalomyelitis, arachnoiditis, vascular lesions of the central nervous system, loss of extremity neuronal function, Charcot-Marie-Tooth disease, Krabbe's disease, leukodystrophies, susceptibility to heart failure, asthma, epilepsy, auditory neurodegeneration, macular degeneration, pigmentary retinitis, and glaucoma-induced optic nerve degeneration; (b) the treatment or prevention of a psychiatric disorder, for example selected from: anxiety disorders (for example, acute stress disorder, panic disorder, agoraphobia, social phobia, specific phobia, obsessive-compulsive disorder, post-traumatic stress disorder, body dysmorphic disorder and generalized anxiety disorder), sexual anxiety disorders (for example, vaginismus, male erectile dysfunction, male orgasmic disorder and female orgasmic disorder), childhood disorders (for example, attention-deficit hyperactivity disorder (ADHD), Asperger's disorder, autistic disorder, conduct disorder, oppositional defiant disorder, separation anxiety disorder and Tourette's disorder), eating disorders (for example, anorexia nervosa and bulimia nervosa), mood disorders (for example, depression, major depressive disorder, bipolar disorder (manic depression), seasonal affective disorder (SAD), cyclothymic disorder and dysthymic disorder), sleeping disorders, cognitive psychiatric disorders (for example, delirium, amnestic disorders), personality disorders (for example, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and obsessive-compulsive personality disorder), psychotic disorders (for example, schizophrenia, delusional disorder, brief psychotic disorder, schizophreniform disorder, schizoaffective disorder and shared psychotic disorder), and substance-related disorders (for example, alcohol dependence, amphetamine dependence, cannabis dependence, cocaine dependence, hallucinogen dependence, inhalant dependence, nicotine dependence, opioid dependence, phencyclidine dependence and sedative dependence); (c) the treatment or prevention of an inflammatory or allergic disorder, for example selected from: cough, pruritus, food intolerance, psoriasis, croup, irritable bowel syndrome, tinnitus, Meniere's disease, stress-induced ulceration or acetylsalicylic acid-induced ulceration, allergic rhinitis, allergic dermatitis, conjunctivitis, inflammation, inflammatory bowel disease, ileitis, pancreatitis, cholecystitis, non-allergic rhinitis, oesophagitis, osteoarthritis, rheumatoid arthritis, hay fever, allergy to house mites, allergy to pet animals, Huntington's disease, acute inflammatory pain, visceral pain, dental pain and headaches, inflammatory hyperalgesia, tactile hyperalgesi, allergic skin reactions, allergic eye reactions, asthma, atherosclerosis, arthritis, chonic ulcers (e.g. chronic vasulitic ulcers associated with rheumatoid arthritis), eczema, maintaining normal breathing, soothing sore throats and coughs, aiding to maintain normal digestion, easing upset stomachs, aiding in the recovery from colds and flu, as a decongestant, soothing headaches, relieving muscle soreness, easing mild aches and pains, providing relief from toothache, providing relief from mouth or stomach ulcers, and maintaining healthy joints; (d) the treatment or prevention of an immune disorder, for example selected from: immunodeficiency conditions such as AIDS, immune hyperactivity conditions and conditions of impaired immune specificity, for example autoimmune diseases such as systemic lupus erythematosus (SLE); and (e) the treatment or prevention of a neoplastic disorder, for example selected from: cancer of the breast, thyroid, colon, lung, ovary, skin, muscle, pancreas, prostate, kidney, reproductive organs, blood, immune system (e.g. spleen, thymus and bone marrow), brain, peripheral nervous system and skin (e.g. melanoma and Kaposi's sarcoma); in a human or non-human mammal in need thereof.

4. A method according to claim 1, wherein the method is used in conjunction with a method for restoring or regenerating neurones, neuronal function or neuronal networks, achieving regeneration or normalisation blood flow to neurones, regrowth and healing of damaged tissues, for example in the post-trauma reconstruction of nerves, tissue grafts, post-surgery reconstruction of nerves, assisting recovery from stroke, TIAs or other ischemia, assisting the healing of wounds, bone and muscle, normalising neuropathic conditions or neuronal abnormalities, or fetal, stem or other cell therapy for increasing the survival rate of transplanted cells, improving the efficiency of surviving cells or a combination thereof.

5. A method according to claim 1, wherein the method is used in conjunction with a method for treating or preventing abnormal behavioral or personality traits.

6. A method according to claim 1, wherein the method is used in conjunction with the assistance of wound healing.

7. A method according to claim 1, wherein the method is used in conjunction with a non-therapeutic method for improving skin, bone, eye, muscle and other tissue health, for example promoting recovery of skin from the effects of ageing, wrinkling or exposure to sun, wind, rain, cold or other damaging media, or a non-therapeutic use to provide for other aspects of health and wellbeing, including recovery of muscle and tissues from exercise, exertion or wasting, improving endurance and reducing the feeling of fatigue.

8. A method according to claim 1, wherein the method is used in conjunction with non-therapeutic methods for the treatment and prevention of neurological and psychiatric conditions that are within the normal range of a population and are not diagnosable disorders.

9. A method according to claim 1, wherein the method is used in a human or animal, being an individual who naturally overexpresses BDNF or GDNF or who is susceptible to the psychiatric side effects of NF-mimicking or stimulating drugs or who is susceptible to receptor- or enzyme-mediated side effects of receptor-(ant)agonistic or enzyme-interacting drugs.

10. A method according to claim 1, wherein the active agent is used without an exogenous administered neurotrophic factor.

11. A method according to claim 1, wherein the method is used in circumstances without clinical control of the administration protocol to the subject.

12. A method according to claim 1, wherein the active agent is selected from sarsasapogenin, smilagenin, episarsasapogenin, epismilagenin, timosaponin BII, metagenin, samogenin, diotigenin, isodiotigenin, texogenin, yonogenin, mexogenin and markogenin and their corresponding ester, ether, ketone and saponin (glycosylated) derivatives.

13. A method according to claim 1, wherein the active agent is selected from sarsasapogenin and smilagenin and their corresponding ester, ether, ketone and saponin (glycosylated) derivatives.

14. A method according to claim 1, wherein the one or more active agent is used in conjunction with one or more co-agent selected from metabolic adjuvants, compounds that increase ketone body levels (ketogenic compounds), the tricarboxylic acid (TCA) cycle intermediates, compounds that are convertible in vivo to TCA intermediates, energy-enhancing compounds, and any mixture thereof.

15. A method according to claim 1, wherein the one or more active agent is administered in a composition comprising the active agent and any suitable additional component, for example, a pharmaceutical composition (medicament), a foodstuff, food supplement or beverage (e.g. a carbonated beverage), or a topical composition such as a cosmetic, eye or skin (e.g. dermatological) composition.

16. A method according to claim 13, wherein the one or more active agent is present in the composition with one or more solubilising and/or suspending and/or dispersing agents to maintain the active agent in solution or suspension or dispersion in the composition, for example medium chain triglycerides (MCTs) or medium chain fatty acids (MCFAs).

17. An agent selected from A/B-cis furostane, furostene, spirostane and spirostene steroidal sapogenins and ester, ether, ketone and glycosylated forms thereof, for use in a method of inducing self-regulated homeostasis of NFs in a subject, by modulating the subject's native NFs in a non-toxic manner under homeostatic control, by administering to the subject an effective amount of one or more such agent.

18. (canceled)

19. A composition comprising at least one agent of claim 17.

20-22. (canceled)

23. A method according to claim 3, wherein the method is used in conjunction with a method for the treatment or prevention of glaucoma-induced optic nerve degeneration.
